SEQUENCE LISTING (67 sequences)

SEQ ID NO: 1 - DNA, 522 bp, Artificial Sequence
  Gamma chain constant region (KC170727.1)
gataaacaacttgatgcagatgtttcccccaagcccactatttttcttccttcaattgctgaaacaaagctccagaaggctggaacatacctttgtcttcttgagaaatttttccctgatgttattaagatacattggcaagaaaagaagagcaacacgattctgggatcccaggaggggaacaccatgaagactaacgacacatacatgaaatttagctggttaacggtgccagaaaagtcactggacaaagaacacagatgtatcgtcagacatgagaataataaaaacggagttgatcaagaaattatctttcctccaataaagacagatgtcatcacaatggatcccaaagacaattgttcaaaagatgcaaatgatacactactgctgcagctcacaaacacctctgcatattacatgtacctcctcctactcctcaagagtgtggtctattttgccatcatcacctgctgtctgcttagaagaacggctttctgctgcaatggagagaaatcataa

SEQ ID NO: 2 - DNA, 465 bp, Artificial Sequence
  Delta chain constant region (X73617.1)
agaagtcagcctcataccaaaccatccgtttttgtcatgaaaaatggaacaaatgtcgcttgtctggtgaaggaattctaccccaaggatataagaataaatctcgtgtcatccaagaagataacagagtttgatcctgctattgtcatctctcccagtgggaagtacaatgctgtcaagcttggtaaatatgaagattcaaattcagtgacatgttcagttcaacacgacaataaaactgtgcactccactgactttgaagtgaagacagattctacagatcacgtaaaaccaaaggaaactgaaaacacaaagcaaccttcaaagagctgccataaacccaaagccatagttcataccgagaaggtgaacatgatgtccctcacagtgcttgggctacgaatgctgtttgcaaagactgttgccgtcaattttctcttgactgccaagttatttttcttgtaa

SEQ ID NO: 3 - DNA, 429 bp, Artificial Sequence
  V.gamma.9 variable region (G115)
atgctgtcactgctgctccacgcatcaacgctggcagtccttggggctctgtgtgtatatggtgcaggtcacctagagcaacctcaaatttccagtactaaaacgctgtcaaaaacagcccgcctggaatgtgtggtgtctggaataacaatttctgcaacatctgtatattggtatcgagagagacctggtgaagtcatacagttcctggtgtccatttcatatgacggcactgtcagaaaggaatccggcattccgtcaggcaaatttgaggtggataggatacctgaaacgtctacatccactctcaccattcacaatgtagagaaacaggacatagctacctactactgtgccttgtgggaggctcaacaggagttgggcaaaaaaatcaaggtatttggtcccggaacaaagcttatcattaca

SEQ ID NO: 4 - DNA, 408 bp, Artificial Sequence
  V.delta.2 variable region (C15)
atgcagaggatctcctccctcatccatctctctctcttctgggcaggagtcatgtcagccattgagttggtgcctgaacaccaaacagtgcctgtgtcaataggggtccctgccaccctcaggtgctccatgaaaggagaagcgatcggtaactactatatcaactggtacaggaagacccaaggtaacacaatcactttcatataccgagaaaaggacatctatggccctggtttcaaagacaatttccaaggtgacattgatattgcaaagaacctggctgtacttaagatacttgcaccatcagagagagatgaagggtcttactactgtgcctgtgacgctctgaagcgcactgataccgataaactcatctttggaaaaggaacccgtgtgactgtggaacca

SEQ ID NO: 5 - DNA, 2411 bp, Artificial Sequence
  V.gamma.9V.delta.2 T cell receptor (V.gamma.9-IRES-V.delta.2) (AGT418)
atgctgtcactgctgctccacgcatcaacgctggcagtccttggggctctgtgtgtatatggtgcaggtcacctagagcaacctcaaatttccagtactaaaacgctgtcaaaaacagcccgcctggaatgtgtggtgtctggaataacaatttctgcaacatctgtatattggtatcgagagagacctggtgaagtcatacagttcctggtgtccatttcatatgacggcactgtcagaaaggaatccggcattccgtcaggcaaatttgaggtggataggatacctgaaacgtctacatccactctcaccattcacaatgtagagaaacaggacatagctacctactactgtgccttgtgggaggctcaacaggagttgggcaaaaaaatcaaggtatttggtcccggaacaaagcttatcattacagataaacaacttgatgcagatgtttcccccaagcccactatttttcttccttcaattgctgaaacaaagctccagaaggctggaacatacctttgtcttcttgagaaatttttccctgatgttattaagatacattggcaagaaaagaagagcaacacgattctgggatcccaggaggggaacaccatgaagactaacgacacatacatgaaatttagctggttaacggtgccagaaaagtcactggacaaagaacacagatgtatcgtcagacatgagaataataaaaacggagttgatcaagaaattatctttcctccaataaagacagatgtcatcacaatggatcccaaagacaattgttcaaaagatgcaaatgatacactactgctgcagctcacaaacacctctgcatattacatgtacctcctcctactcctcaagagtgtggtctattttgccatcatcacctgctgtctgcttagaagaacggctttctgctgcaatggagagaaatcataagcggccgcccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccgctagcatgcagaggatctcctccctcatccatctctctctcttctgggcaggagtcatgtcagccattgagttggtgcctgaacaccaaacagtgcctgtgtcaataggggtccctgccaccctcaggtgctccatgaaaggagaagcgatcggtaactactatatcaactggtacaggaagacccaaggtaacacaatcactttcatataccgagaaaaggacatctatggccctggtttcaaagacaatttccaaggtgacattgatattgcaaagaacctggctgtacttaagatacttgcaccatcagagagagatgaagggtcttactactgtgcctgtgacgctctgaagcgcactgataccgataaactcatctttggaaaaggaacccgtgtgactgtggaaccaagaagtcagcctcataccaaaccatccgtttttgtcatgaaaaatggaacaaatgtcgcttgtctggtgaaggaattctaccccaaggatataagaataaatctcgtgtcatccaagaagataacagagtttgatcctgctattgtcatctctcccagtgggaagtacaatgctgtcaagcttggtaaatatgaagattcaaattcagtgacatgttcagttcaacacgacaataaaactgtgcactccactgactttgaagtgaagacagattctacagatcacgtaaaaccaaaggaaactgaaaacacaaagcaaccttcaaagagctgccataaacccaaagccatagttcataccgagaaggtgaacatgatgtccctcacagtgcttgggctacgaatgctgtttgcaaagactgttgccgtcaattttctcttgactgccaagttatttttcttgtaa

SEQ ID NO: 6 - DNA, 573 bp, Artificial Sequence
  IRES sequence
ccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaacc

SEQ ID NO: 7 - DNA, 1104 bp, Artificial Sequence
  EF1.alpha. promoter
ccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 8 - DNA, 350 bp, Artificial Sequence
  CMV promoter
actagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaaga

SEQ ID NO: 9 - DNA, 2370 bp, Artificial Sequence
  CD3 (Gamma-Delta-Epsilon-Zeta chain)
atggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgagggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgccagtcgagagcttcagacaagcagactctgttgcccaatgaccagctctaccagcccctcaaggatcgagaagatgaccaatatagtcatctacaaggaaaccagttgaggaggaatggaccggtgaaacagactttgaattttgaccttctcaagttggcgggagacgtggagtccaacccagggcccatggaacatagcacgtttctctctggcctggtactggctacccttctctcgcaagtgagccccttcaagatacctatagaggaacttgaggacagagtgtttgtgaattgcaataccagcatcacatgggtagagggaacggtgggaacactgctctcagacattacaagactggacctgggaaaacgcatcctggacccacgaggaatatataggtgtaatgggacagatatatacaaggacaaagaatctaccgtgcaagttcattatcgaatgtgccagagctgtgtggagctggatccagccaccgtggctggcatcattgtcactgatgtcattgccactctgctccttgctttgggagtcttctgctttgctggacatgagactggaaggctgtctggggctgccgacacacaagctctgttgaggaatgaccaggtctatcagcccctccgagatcgagatgatgctcagtacagccaccttggaggaaactgggctcggaacaagagatctgagggcagaggaagtctgctaacatgcggtgacgtcgaggagaatcctggcccaatgcagtcgggcactcactggagagttctgggcctctgcctcttatcagttggcgtttgggggcaagatggtaatgaagaaatgggtggtattacacagacaccatataaagtctccatctctggaaccacagtaatattgacatgccctcagtatcctggatctgaaatactatggcaacacaacgacaagaatattggaggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtggccacaattgtcatagtggacatctgcatcactgggggcttgctgctgctggtttactactggagcaagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgaatcagagacgcatcggatccgccacgaacttctctctgttaaagcaagcaggagacgtggaagaaaaccccggtcccatgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 10 - DNA, 352 bp, Artificial Sequence
  Helper/Rev - CMV early (CAG) enhancer - Enhance Transcription
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatc

SEQ ID NO: 11 - DNA, 290 bp, Artificial Sequence
  Helper/Rev - Chicken beta actin (CAG) promoter - Transcription
gctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 12 - DNA, 960 bp, Artificial Sequence
  Helper/Rev - Chicken beta actin intron - Enhance gene expression
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcgg

SEQ ID NO: 13 - DNA, 1503 bp, Artificial Sequence
  Helper/Rev - HIV Gag - Viral capsid
atgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataa

SEQ ID NO: 14 - DNA, 1872 bp, Artificial Sequence
  Helper/Rev - HIV Pol - Protease and reverse transcriptase
atgaatttgccaggaagatggaaaccaaaaatgatagggggaattggaggttttatcaaagtaggacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaaatatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagatgggttggtcagtgctggaatcaggaaagtacta

SEQ ID NO: 15 - DNA, 867 bp, Artificial Sequence
  Helper Rev - HIV Integrase - Integration of viral RNA
tttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaa

SEQ ID NO: 16 - DNA, 234 bp, Artificial Sequence
  Helper/Rev - HIV RRE - Binds Rev element
aggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcct

SEQ ID NO: 17 - DNA, 351 bp, Artificial Sequence
  Helper/Rev - HIV Rev - Nuclear export and stabilize viral mRNA
atggcaggaagaagcggagacagcgacgaagaactcctcaaggcagtcagactcatcaagtttctctatcaaagcaacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatag

SEQ ID NO: 18 - DNA, 448 bp, Artificial Sequence
  Helper/Rev - Rabbit beta globin poly A - RNA stability
agatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatc

SEQ ID NO: 19 - DNA, 577 bp, Artificial Sequence
  Envelope - CMV promoter - Transcription
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagc

SEQ ID NO: 20 - DNA, 573 bp, Artificial Sequence
  Envelope - Beta globin intron - Enhance gene expression
gtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctctttcctacag

SEQ ID NO: 21 - DNA, 450 bp, Artificial Sequence
  Envelope - Rabbit beta globin poly A - RNA stability
agatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 22 - DNA, 31 bp, Artificial Sequence
  Primer
taagcagaattcatgaatttgccaggaagat

SEQ ID NO: 23 - DNA, 36 bp, Artificial Sequence
  Primer
ccatacaatgaatggacactaggcggccgcacgaat

SEQ ID NO: 24 - DNA, 2745 bp, Artificial Sequence
  Gag, Pol, Integrase fragment
gaattcatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaa

SEQ ID NO: 25 - DNA, 1586 bp, Artificial Sequence
  DNA Fragment containing Rev, RRE and rabbit beta globin poly A
tctagaatggcaggaagaagcggagacagcgacgaagagctcatcagaacagtcagactcatcaagcttctctatcaaagcaacccacctcccaatcccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatagaggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcagcggccgccccggg

SEQ ID NO: 26 - DNA, 1614 bp, Artificial Sequence
  DNA fragment containing the CAG enhancer/promoter/intron sequence
acgcgttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcgggaattc

SEQ ID NO: 27 - DNA, 1531 bp, Artificial Sequence
  DNA fragment containing VSV-G
gaattcatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgagaattc

SEQ ID NO: 28 - DNA, 395 bp, Artificial Sequence
  Helper/Rev - RSV promoter - Transcription
cgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatacacttgtagtcttgcaacatggtaacgatgagttagcaacatgccttacaaggagagaaaaagcaccgtgcatgccgattggtggaagtaaggtggtacgatcgtgccttattaggaaggcaacagacaggtctgacatggattggacgaaccactgaattccgcattgcagagataattgtatttaagtgcctagctcgatacaataaacgccatttgaccattcaccacattggtgtgcacct

SEQ ID NO: 29 - DNA, 351 bp, Artificial Sequence; identical to SEQ ID NO: 17
  Helper/Rev - HIV Rev - Nuclear export and stabilize viral mRNA

SEQ ID NO: 30 - DNA, 884 bp, Artificial Sequence
  RSV promoter and HIV Rev
caattgcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatacacttgtagtcttgcaacatggtaacgatgagttagcaacatgccttacaaggagagaaaaagcaccgtgcatgccgattggtggaagtaaggtggtacgatcgtgccttattaggaaggcaacagacaggtctgacatggattggacgaaccactgaattccgcattgcagagataattgtatttaagtgcctagctcgatacaataaacgccatttgaccattcaccacattggtgtgcacctccaagctcgagctcgtttagtgaaccgtcagatcgcctggagacgccatccacgctgttttgacctccatagaagacaccgggaccgatccagcctcccctcgaagctagcgattaggcatctcctatggcaggaagaagcggagacagcgacgaagaactcctcaaggcagtcagactcatcaagtttctctatcaaagcaacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatagtctaga

SEQ ID NO: 31 - DNA, 1104 bp, Artificial Sequence; identical to SEQ ID NO: 7
  Elongation Factor-1 alpha (EF1-alpha) promoter

SEQ ID NO: 32 - DNA, 511 bp, Artificial Sequence
  Promoter - PGK
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 33 - DNA, 1162 bp, Artificial Sequence
  Promoter - UbC
gcgccgggttttggcgcctcccgcgggcgcccccctcctcacggcgagcgctgccacgtcagacgaagggcgcaggagcgttcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagttgcgggctgctgggctggccggggctttcgtggccgccgggccgctcggtgggacggaagcgtgtggagagaccgccaagggctgtagtctgggtccgcgagcaaggttgccctgaactgggggttggggggagcgcacaaaatggcggctgttcccgagtcttgaatggaagacgcttgtaaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcgggtttgtcgtctggttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaa

SEQ ID NO: 34 - DNA, 120 bp, Artificial Sequence
  Poly A - SV40
gtttattgcagcttataatggttacaaataaagcaatagcatcacaaatttcacaaataaagcatttttttcactgcattctagttgtggtttgtccaaactcatcaatgtatcttatca

SEQ ID NO: 35 - DNA, 227 bp, Artificial Sequence
  Poly A - bGH
gactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 36 - DNA, 1695 bp, Artificial Sequence
  Envelope - RD114
atgaaactcccaacaggaatggtcattttatgtagcctaataatagttcgggcagggtttgacgacccccgcaaggctatcgcattagtacaaaaacaacatggtaaaccatgcgaatgcagcggagggcaggtatccgaggccccaccgaactccatccaacaggtaacttgcccaggcaagacggcctacttaatgaccaaccaaaaatggaaatgcagagtcactccaaaaaatctcacccctagcgggggagaactccagaactgcccctgtaacactttccaggactcgatgcacagttcttgttatactgaataccggcaatgcagggcgaataataagacatactacacggccaccttgcttaaaatacggtctgggagcctcaacgaggtacagatattacaaaaccccaatcagctcctacagtccccttgtaggggctctataaatcagcccgtttgctggagtgccacagcccccatccatatctccgatggtggaggacccctcgatactaagagagtgtggacagtccaaaaaaggctagaacaaattcataaggctatgcatcctgaacttcaataccaccccttagccctgcccaaagtcagagatgaccttagccttgatgcacggacttttgatatcctgaataccacttttaggttactccagatgtccaattttagccttgcccaagattgttggctctgtttaaaactaggtacccctacccctcttgcgatacccactccctctttaacctactccctagcagactccctagcgaatgcctcctgtcagattatacctcccctcttggttcaaccgatgcagttctccaactcgtcctgtttatcttcccctttcattaacgatacggaacaaatagacttaggtgcagtcacctttactaactgcacctctgtagccaatgtcagtagtcctttatgtgccctaaacgggtcagtcttcctctgtggaaataacatggcatacacctatttaccccaaaactggacaggactttgcgtccaagcctccctcctccccgacattgacatcatcccgggggatgagccagtccccattcctgccattgatcattatatacatagacctaaacgagctgtacagttcatccctttactagctggactgggaatcaccgcagcattcaccaccggagctacaggcctaggtgtctccgtcacccagtatacaaaattatcccatcagttaatatctgatgtccaagtcttatccggtaccatacaagatttacaagaccaggtagactcgttagctgaagtagttctccaaaataggaggggactggacctactaacggcagaacaaggaggaatttgtttagccttacaagaaaaatgctgtttttatgctaacaagtcaggaattgtgagaaacaaaataagaaccctacaagaagaattacaaaaacgcagggaaagcctggcatccaaccctctctggaccgggctgcagggctttcttccgtacctcctacctctcctgggacccctactcaccctcctactcatactaaccattgggccatgcgttttcaatcgattggtccaatttgttaaagacaggatctcagtggtccaggctctggttttgactcagcaatatcaccagctaaaacccatagagtacgagccatga

SEQ ID NO: 37 - DNA, 2013 bp, Artificial Sequence
  Envelope - GALV
atgcttctcacctcaagcccgcaccaccttcggcaccagatgagtcctgggagctggaaaagactgatcatcctcttaagctgcgtattcggagacggcaaaacgagtctgcagaataagaacccccaccagcctgtgaccctcacctggcaggtactgtcccaaactggggacgttgtctgggacaaaaaggcagtccagcccctttggacttggtggccctctcttacacctgatgtatgtgccctggcggccggtcttgagtcctgggatatcccgggatccgatgtatcgtcctctaaaagagttagacctcctgattcagactatactgccgcttataagcaaatcacctggggagccatagggtgcagctaccctcgggctaggaccaggatggcaaattcccccttctacgtgtgtccccgagctggccgaacccattcagaagctaggaggtgtggggggctagaatccctatactgtaaagaatggagttgtgagaccacgggtaccgtttattggcaacccaagtcctcatgggacctcataactgtaaaatgggaccaaaatgtgaaatgggagcaaaaatttcaaaagtgtgaacaaaccggctggtgtaaccccctcaagatagacttcacagaaaaaggaaaactctccagagattggataacggaaaaaacctgggaattaaggttctatgtatatggacacccaggcatacagttgactatccgcttagaggtcactaacatgccggttgtggcagtgggcccagaccctgtccttgcggaacagggacctcctagcaagcccctcactctccctctctccccacggaaagcgccgcccacccctctacccccggcggctagtgagcaaacccctgcggtgcatggagaaactgttaccctaaactctccgcctcccaccagtggcgaccgactctttggccttgtgcagggggccttcctaaccttgaatgctaccaacccaggggccactaagtcttgctggctctgtttgggcatgagccccccttattatgaagggatagcctcttcaggagaggtcgcttatacctccaaccatacccgatgccactggggggcccaaggaaagcttaccctcactgaggtctccggactcgggtcatgcatagggaaggtgcctcttacccatcaacatctttgcaaccagaccttacccatcaattcctctaaaaaccatcagtatctgctcccctcaaaccatagctggtgggcctgcagcactggcctcaccccctgcctctccacctcagtttttaatcagtctaaagacttctgtgtccaggtccagctgatcccccgcatctattaccattctgaagaaaccttgttacaagcctatgacaaatcaccccccaggtttaaaagagagcctgcctcacttaccctagctgtcttcctggggttagggattgcggcaggtataggtactggctcaaccgccctaattaaagggcccatagacctccagcaaggcctaaccagcctccaaatcgccattgacgctgacctccgggcccttcaggactcaatcagcaagctagaggactcactgacttccctatctgaggtagtactccaaaataggagaggccttgacttactattccttaaagaaggaggcctctgcgcggccctaaaagaagagtgctgtttttatgtagaccactcaggtgcagtacgagactccatgaaaaaacttaaagaaagactagataaaagacagttagagcgccagaaaaaccaaaactggtatgaagggtggttcaataactccccttggtttactaccctactatcaaccatcgctgggcccctattgctcctccttttgttactcactcttgggccctgcatcatcaataaattaatccaattcatcaatgataggataagtgcagtcaaaattttagtccttagacagaaatatcagaccctagataacgaggaaaacctttaa

SEQ ID NO: 38 - DNA, 1530 bp, Artificial Sequence
  Envelope - FUG
atggttccgcaggttcttttgtttgtactccttctgggtttttcgttgtgtttcgggaagttccccatttacacgataccagacgaacttggtccctggagccctattgacatacaccatctcagctgtccaaataacctggttgtggaggatgaaggatgtaccaacctgtccgagttctcctacatggaactcaaagtgggatacatctcagccatcaaagtgaacgggttcacttgcacaggtgttgtgacagaggcagagacctacaccaactttgttggttatgtcacaaccacattcaagagaaagcatttccgccccaccccagacgcatgtagagccgcgtataactggaagatggccggtgaccccagatatgaagagtccctacacaatccataccccgactaccactggcttcgaactgtaagaaccaccaaagagtccctcattatcatatccccaagtgtgacagatttggacccatatgacaaatcccttcactcaagggtcttccctggcggaaagtgctcaggaataacggtgtcctctacctactgctcaactaaccatgattacaccatttggatgcccgagaatccgagaccaaggacaccttgtgacatttttaccaatagcagagggaagagagcatccaacgggaacaagacttgcggctttgtggatgaaagaggcctgtataagtctctaaaaggagcatgcaggctcaagttatgtggagttcttggacttagacttatggatggaacatgggtcgcgatgcaaacatcagatgagaccaaatggtgccctccagatcagttggtgaatttgcacgactttcgctcagacgagatcgagcatctcgttgtggaggagttagttaagaaaagagaggaatgtctggatgcattagagtccatcatgaccaccaagtcagtaagtttcagacgtctcagtcacctgagaaaacttgtcccagggtttggaaaagcatataccatattcaacaaaaccttgatggaggctgatgctcactacaagtcagtccggacctggaatgagatcatcccctcaaaagggtgtttgaaagttggaggaaggtgccatcctcatgtgaacggggtgtttttcaatggtataatattagggcctgacgaccatgtcctaatcccagagatgcaatcatccctcctccagcaacatatggagttgttggaatcttcagttatccccctgatgcaccccctggcagacccttctacagttttcaaagaaggtgatgaggctgaggattttgttgaagttcacctccccgatgtgtacaaacagatctcaggggttgacctgggtctcccgaactggggaaagtatgtattgatgactgcaggggccatgattggcctggtgttgatattttccctaatgacatggtgcagagttggtatccatctttgcattaaattaaagcacaccaagaaaagacagatttatacagacatagagatgaaccgacttggaaagtaa

SEQ ID NO: 39 - DNA, 1497 bp, Artificial Sequence
  Envelope - LCMV
atgggtcagattgtgacaatgtttgaggctctgcctcacatcatcgatgaggtgatcaacattgtcattattgtgcttatcgtgatcacgggtatcaaggctgtctacaattttgccacctgtgggatattcgcattgatcagtttcctacttctggctggcaggtcctgtggcatgtacggtcttaagggacccgacatttacaaaggagtttaccaatttaagtcagtggagtttgatatgtcacatctgaacctgaccatgcccaacgcatgttcagccaacaactcccaccattacatcagtatggggacttctggactagaattgaccttcaccaatgattccatcatcagtcacaacttttgcaatctgacctctgccttcaacaaaaagacctttgaccacacactcatgagtatagtttcgagcctacacctcagtatcagagggaactccaactataaggcagtatcctgcgacttcaacaatggcataaccatccaatacaacttgacattctcagatcgacaaagtgctcagagccagtgtagaaccttcagaggtagagtcctagatatgtttagaactgccttcggggggaaatacatgaggagtggctggggctggacaggctcagatggcaagaccacctggtgtagccagacgagttaccaatacctgattatacaaaatagaacctgggaaaaccactgcacatatgcaggtccttttgggatgtccaggattctcctttcccaagagaagactaagttcttcactaggagactagcgggcacattcacctggactttgtcagactcttcaggggtggagaatccaggtggttattgcctgaccaaatggatgattcttgctgcagagcttaagtgtttcgggaacacagcagttgcgaaatgcaatgtaaatcatgatgccgaattctgtgacatgctgcgactaattgactacaacaaggctgctttgagtaagttcaaagaggacgtagaatctgccttgcacttattcaaaacaacagtgaattctttgatttcagatcaactactgatgaggaaccacttgagagatctgatgggggtgccatattgcaattactcaaagttttggtacctagaacatgcaaagaccggcgaaactagtgtccccaagtgctggcttgtcaccaatggttcttacttaaatgagacccacttcagtgatcaaatcgaacaggaagccgataacatgattacagagatgttgaggaaggattacataaagaggcaggggagtacccccctagcattgatggaccttctgatgttttccacatctgcatatctagtcagcatcttcctgcaccttgtcaaaataccaacacacaggcacataaaaggtggctcatgtccaaagccacaccgattaaccaacaaaggaatttgtagttgtggtgcatttaaggtgcctggtgtaaaaaccgtctggaaaagacgctga

SEQ ID NO: 40 - DNA, 1692 bp, Artificial Sequence
  Envelope - FPV
atgaacactcaaatcctggttttcgcccttgtggcagtcatccccacaaatgcagacaaaatttgtcttggacatcatgctgtatcaaatggcaccaaagtaaacacactcactgagagaggagtagaagttgtcaatgcaacggaaacagtggagcggacaaacatccccaaaatttgctcaaaagggaaaagaaccactgatcttggccaatgcggactgttagggaccattaccggaccacctcaatgcgaccaatttctagaattttcagctgatctaataatcgagagacgagaaggaaatgatgtttgttacccggggaagtttgttaatgaagaggcattgcgacaaatcctcagaggatcaggtgggattgacaaagaaacaatgggattcacatatagtggaataaggaccaacggaacaactagtgcatgtagaagatcagggtcttcattctatgcagaaatggagtggctcctgtcaaatacagacaatgctgctttcccacaaatgacaaaatcatacaaaaacacaaggagagaatcagctctgatagtctggggaatccaccattcaggatcaaccaccgaacagaccaaactatatgggagtggaaataaactgataacagtcgggagttccaaatatcatcaatcttttgtgccgagtccaggaacacgaccgcagataaatggccagtccggacggattgattttcattggttgatcttggatcccaatgatacagttacttttagtttcaatggggctttcatagctccaaatcgtgccagcttcttgaggggaaagtccatggggatccagagcgatgtgcaggttgatgccaattgcgaaggggaatgctaccacagtggagggactataacaagcagattgccttttcaaaacatcaatagcagagcagttggcaaatgcccaagatatgtaaaacaggaaagtttattattggcaactgggatgaagaacgttcccgaaccttccaaaaaaaggaaaaaaagaggcctgtttggcgctatagcagggtttattgaaaatggttgggaaggtctggtcgacgggtggtacggtttcaggcatcagaatgcacaaggagaaggaactgcagcagactacaaaagcacccaatcggcaattgatcagataaccggaaagttaaatagactcattgagaaaaccaaccagcaatttgagctaatagataatgaattcactgaggtggaaaagcagattggcaatttaattaactggaccaaagactccatcacagaagtatggtcttacaatgctgaacttcttgtggcaatggaaaaccagcacactattgatttggctgattcagagatgaacaagctgtatgagcgagtgaggaaacaattaagggaaaatgctgaagaggatggcactggttgctttgaaatttttcataaatgtgacgatgattgtatggctagtataaggaacaatacttatgatcacagcaaatacagagaagaagcgatgcaaaatagaatacaaattgacccagtcaaattgagtagtggctacaaagatgtgatactttggtttagcttcggggcatcatgctttttgcttcttgccattgcaatgggccttgttttcatatgtgtgaagaacggaaacatgcggtgcactatttgtatataa

SEQ ID NO: 41 - DNA, 1266 bp, Artificial Sequence
  Envelope - RRV
agtgtaacagagcactttaatgtgtataaggctactagaccatacctagcacattgcgccgattgcggggacgggtacttctgctatagcccagttgctatcgaggagatccgagatgaggcgtctgatggcatgcttaagatccaagtctccgcccaaataggtctggacaaggcaggcacccacgcccacacgaagctccgatatatggctggtcatgatgttcaggaatctaagagagattccttgagggtgtacacgtccgcagcgtgctccatacatgggacgatgggacacttcatcgtcgcacactgtccaccaggcgactacctcaaggtttcgttcgaggacgcagattcgcacgtgaaggcatgtaaggtccaatacaagcacaatccattgccggtgggtagagagaagttcgtggttagaccacactttggcgtagagctgccatgcacctcataccagctgacaacggctcccaccgacgaggagattgacatgcatacaccgccagatataccggatcgcaccctgctatcacagacggcgggcaacgtcaaaataacagcaggcggcaggactatcaggtacaactgtacctgcggccgtgacaacgtaggcactaccagtactgacaagaccatcaacacatgcaagattgaccaatgccatgctgccgtcaccagccatgacaaatggcaatttacctctccatttgttcccagggctgatcagacagctaggaaaggcaaggtacacgttccgttccctctgactaacgtcacctgccgagtgccgttggctcgagcgccggatgccacctatggtaagaaggaggtgaccctgagattacacccagatcatccgacgctcttctcctataggagtttaggagccgaaccgcacccgtacgaggaatgggttgacaagttctctgagcgcatcatcccagtgacggaagaagggattgagtaccagtggggcaacaacccgccggtctgcctgtgggcgcaactgacgaccgagggcaaaccccatggctggccacatgaaatcattcagtactattatggactataccccgccgccactattgccgcagtatccggggcgagtctgatggccctcctaactctggcggccacatgctgcatgctggccaccgcgaggagaaagtgcctaacaccgtacgccctgacgccaggagcggtggtaccgttgacactggggctgctttgctgcgcaccgagggcgaatgca

SEQ ID NO: 42 - DNA, 1938 bp, Artificial Sequence
  Envelope - MLV 10A1
atggaaggtccagcgttctcaaaaccccttaaagataagattaacccgtggaagtccttaatggtcatgggggtctatttaagagtagggatggcagagagcccccatcaggtctttaatgtaacctggagagtcaccaacctgatgactgggcgtaccgccaatgccacctcccttttaggaactgtacaagatgccttcccaagattatattttgatctatgtgatctggtcggagaagagtgggacccttcagaccaggaaccatatgtcgggtatggctgcaaataccccggagggagaaagcggacccggacttttgacttttacgtgtgccctgggcataccgtaaaatcggggtgtggggggccaagagagggctactgtggtgaatggggttgtgaaaccaccggacaggcttactggaagcccacatcatcatgggacctaatctcccttaagcgcggtaacaccccctgggacacgggatgctccaaaatggcttgtggcccctgctacgacctctccaaagtatccaattccttccaaggggctactcgagggggcagatgcaaccctctagtcctagaattcactgatgcaggaaaaaaggctaattgggacgggcccaaatcgtggggactgagactgtaccggacaggaacagatcctattaccatgttctccctgacccgccaggtcctcaatatagggccccgcatccccattgggcctaatcccgtgatcactggtcaactacccccctcccgacccgtgcagatcaggctccccaggcctcctcagcctcctcctacaggcgcagcctctatagtccctgagactgccccaccttctcaacaacctgggacgggagacaggctgctaaacctggtagaaggagcctatcaggcgcttaacctcaccaatcccgacaagacccaagaatgttggctgtgcttagtgtcgggacctccttattacgaaggagtagcggtcgtgggcacttataccaatcattctaccgccccggccagctgtacggccacttcccaacataagcttaccctatctgaagtgacaggacagggcctatgcatgggagcactacctaaaactcaccaggccttatgtaacaccacccaaagtgccggctcaggatcctactaccttgcagcacccgctggaacaatgtgggcttgtagcactggattgactccctgcttgtccaccacgatgctcaatctaaccacagactattgtgtattagttgagctctggcccagaataatttaccactcccccgattatatgtatggtcagcttgaacagcgtaccaaatataagagggagccagtatcgttgaccctggcccttctgctaggaggattaaccatgggagggattgcagctggaatagggacggggaccactgccctaatcaaaacccagcagtttgagcagcttcacgccgctatccagacagacctcaacgaagtcgaaaaatcaattaccaacctagaaaagtcactgacctcgttgtctgaagtagtcctacagaaccgaagaggcctagatttgctcttcctaaaagagggaggtctctgcgcagccctaaaagaagaatgttgtttttatgcagaccacacgggactagtgagagacagcatggccaaactaagggaaaggcttaatcagagacaaaaactatttgagtcaggccaaggttggttcgaagggcagtttaatagatccccctggtttaccaccttaatctccaccatcatgggacctctaatagtactcttactgatcttactctttggaccctgcattctcaatcgattggtccaatttgttaaagacaggatctcagtggtccaggctctggttttgactcaacaatatcaccagctaaaacctatagagtacgagccatga

SEQ ID NO: 43 - DNA, 2030 bp, Artificial Sequence
  Envelope - Ebola
atgggtgttacaggaatattgcagttacctcgtgatcgattcaagaggacatcattctttctttgggtaattatccttttccaaagaacattttccatcccacttggagtcatccacaatagcacattacaggttagtgatgtcgacaaactggtttgccgtgacaaactgtcatccacaaatcaattgagatcagttggactgaatctcgaagggaatggagtggcaactgacgtgccatctgcaactaaaagatggggcttcaggtccggtgtcccaccaaaggtggtcaattatgaagctggtgaatgggctgaaaactgctacaatcttgaaatcaaaaaacctgacgggagtgagtgtctaccagcagcgccagacgggattcggggcttcccccggtgccggtatgtgcacaaagtatcaggaacgggaccgtgtgccggagactttgccttccacaaagagggtgctttcttcctgtatgaccgacttgcttccacagttatctaccgaggaacgactttcgctgaaggtgtcgttgcatttctgatactgccccaagctaagaaggacttcttcagctcacaccccttgagagagccggtcaatgcaacggaggacccgtctagtggctactattctaccacaattagatatcaagctaccggttttggaaccaatgagacagagtatttgttcgaggttgacaatttgacctacgtccaacttgaatcaagattcacaccacagtttctgctccagctgaatgagacaatatatacaagtgggaaaaggagcaataccacgggaaaactaatttggaaggtcaaccccgaaattgatacaacaatcggggagtgggccttctgggaaactaaaaaaacctcactagaaaaattcgcagtgaagagttgtctttcacagctgtatcaaacagagccaaaaacatcagtggtcagagtccggcgcgaacttcttccgacccagggaccaacacaacaactgaagaccacaaaatcatggcttcagaaaattcctctgcaatggttcaagtgcacagtcaaggaagggaagctgcagtgtcgcatctgacaacccttgccacaatctccacgagtcctcaaccccccacaaccaaaccaggtccggacaacagcacccacaatacacccgtgtataaacttgacatctctgaggcaactcaagttgaacaacatcaccgcagaacagacaacgacagcacagcctccgacactccccccgccacgaccgcagccggacccctaaaagcagagaacaccaacacgagcaagggtaccgacctcctggaccccgccaccacaacaagtccccaaaaccacagcgagaccgctggcaacaacaacactcatcaccaagataccggagaagagagtgccagcagcgggaagctaggcttaattaccaatactattgctggagtcgcaggactgatcacaggcgggaggagagctcgaagagaagcaattgtcaatgctcaacccaaatgcaaccctaatttacattactggactactcaggatgaaggtgctgcaatcggactggcctggataccatatttcgggccagcagccgagggaatttacatagaggggctgatgcacaatcaagatggtttaatctgtgggttgagacagctggccaacgagacgactcaagctcttcaactgttcctgagagccacaaccgagctacgcaccttttcaatcctcaaccgtaaggcaattgatttcttgctgcagcgatggggcggcacatgccacattttgggaccggactgctgtatcgaaccacatgattggaccaagaacataacagacaaaattgatcagattattcatgattttgttgataaaacccttccggaccagggggacaatgacaattggtggacaggatggagacaatggataccggcaggtattggagttacaggcgttataattgcagttatcgctttattctgtatatgcaaatttgtcttttag

SEQ ID NO: 44 - DNA, 228 bp, Artificial Sequence
  Rous Sarcoma virus (RSV) promoter
gtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacg

SEQ ID NO: 45 - DNA, 180 bp, Artificial Sequence
  5 prime Long terminal repeat (LTR)
ggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 46 - DNA, 41 bp, Artificial Sequence
  Psi Packaging signal
tacgccaaaaattttgactagcggaggctagaaggagagag

SEQ ID NO: 47 - DNA, 233 bp, Artificial Sequence
  Helper/Rev response element (RRE)
aggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcc

SEQ ID NO: 48 - DNA, 118 bp, Artificial Sequence
  Central polypurine tract (cPPT)
ttttaaaagaaaaggggggattggggggtacagtgcaggggaaagaatagtagacataatagcaacagacatacaaactaaagaattacaaaaacaaattacaaaattcaaaatttta

SEQ ID NO: 49 - DNA, 217 bp, Artificial Sequence
  Polymerase III shRNA promoters - H1 promoter
gaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactt

SEQ ID NO: 50 - DNA, 53 bp, Artificial Sequence
  FDPS shRNA sequence #1
gtcctggagtacaatgccattctcgagaatggcattgtactccaggacttttt

SEQ ID NO: 51 - DNA, 53 bp, Artificial Sequence
  FDPS shRNA sequence #2
gcaggatttcgttcagcacttctcgagaagtgctgaacgaaatcctgcttttt

SEQ ID NO: 52 - DNA, 53 bp, Artificial Sequence
  FDPS shRNA sequence #3
gccatgtacatggcaggaattctcgagaattcctgccatgtacatggcttttt

SEQ ID NO: 53 - DNA, 53 bp, Artificial Sequence
  FDPS shRNA sequence #4
gcagaaggaggctgagaaagtctcgagactttctcagcctccttctgcttttt

SEQ ID NO: 54 - DNA, 590 bp, Artificial Sequence
  Long WPRE sequence
aatcaacctctgattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcct

SEQ ID NO: 55 - DNA, 250 bp, Artificial Sequence
  3 prime delta LTR
tggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtca

SEQ ID NO: 56 - DNA, 290 bp, Artificial Sequence; identical to SEQ ID NO: 11
  Helper/Rev - Chicken beta actin (CAG) promoter - Transcription

SEQ ID NO: 57 - DNA, 1503 bp, Artificial Sequence; identical to SEQ ID NO: 13
  Helper/Rev - HIV Gag - Viral capsid

SEQ ID NO: 58 - DNA, 1872 bp, Artificial Sequence; identical to SEQ ID NO: 14
  Helper/Rev - HIV Pol - Protease and reverse transcriptase

SEQ ID NO: 59 - DNA, 867 bp, Artificial Sequence; identical to SEQ ID NO: 15
  Helper Rev - HIV Integrase - Integration of viral RNA

SEQ ID NO: 60 - DNA, 234 bp, Artificial Sequence; identical to SEQ ID NO: 16
  Helper/Rev - HIV RRE - Binds Rev element

SEQ ID NO: 61 - DNA, 351 bp, Artificial Sequence; identical to SEQ ID NO: 17
  Helper/Rev - HIV Rev - Nuclear export and stabilize viral mRNA

SEQ ID NO: 62 - DNA, 577 bp, Artificial Sequence; identical to SEQ ID NO: 19
  Envelope - CMV promoter - Transcription

SEQ ID NO: 63 - DNA, 1519 bp, Artificial Sequence
  Envelope - VSV-G - Glycoprotein envelope-cell entry
atgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatga

SEQ ID NO: 64 - DNA, 116 bp, Artificial Sequence
  miR30 FDPS sequence #1
aaggtatattgctgttgacagtgagcgacactttctcagcctccttctgcgtgaagccacagatggcagaaggaggctgagaaagtgctgcctactgcctcggacttcaaggggct

SEQ ID NO: 65 - DNA, 114 bp, Artificial Sequence
  miR30 FDPS sequence #2
aaggtatattgctgttgacagtgagcgacactttctcagcctccttctgcgtgaagccacagatggcagaagggctgagaaagtgctgcctactgcctcggacttcaaggggct

SEQ ID NO: 66 - DNA, 91 bp, Artificial Sequence
  miR30 FDPS sequence #3
tgctgttgacagtgagcgactttctcagcctccttctgcgtgaagccacagatggcagaaggaggctgagaaagttgcctactgcctcgga

SEQ ID NO: 67 - DNA, 1284 bp, Artificial Sequence
  DNA Fragment containing RRE and rabbit beta globin poly A
gcggccgccacatggaattctgcaacaactgctgtttatccatttcagaattggaggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttgctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctacgaatga

(19) United States
(12) Patent Application Publication
(10) Pub. No.: US 2024/0075140 A1
(43) Pub. Date: Mar. 7, 2024

(54) ENGINEERED NK CELLS AND METHODS OF TREATING CANCER

(71) Applicant: American Gene Technologies Intemational Inc, Rockville, MD (US)

(72) Inventors: Haishan Li, Rockville, MD (US); Tyler Lahusen, Rockville, MD (US); Charles David Pauza, Rockville, MD (US)

(21) Appl. No.: 18/271,632

(22) PCT Filed: Jan. 21, 2022
(86) PCT No.: PCT/US2022/013422

§ 371 Date: Jul. 10, 2023

Related U.S. Application Data
(60) Provisional application No. 63/140,118, filed on Jan. 21, 2021.

(51) Int. Cl. A61K 39/00; A61K 31/675; A61P 35/00; A61P 35/02; C07K 14/725; C12N 5/0783; C12N 15/113; C12N 15/86

(52) U.S. Cl. CPC A61K 39/4613; A61K 31/675; A61K 39/4632; A61K 39/4644; A61P 35/00; A61P 35/02; C07K 14/7051; C12N 5/0646; C12N 15/1137; C12N 15/1138; C12N 15/86; A61K 2239/48; C12N 2310/14; C12N 2740/15043

(57) ABSTRACT

The present disclosure relates generally to modified NK cell compositions and methods of using the modified cell compositions in immunotherapy applications. In embodiments, the modified cell express a γ chain and a δ chain. In embodiments, the modified NK cell compositions can be used to treat various cancers.

### CROSS-REFERENCE TO RELATED APPLICATION

This application claims priority to U.S. Provisional Patent Application No. 63/140,118, filed Jan. 21, 2021, entitled “ENGINEERED NK CELLS AND METHODS OF TREATING CANCER,” the disclosure of which is incorporated herein by reference.

### FIELD

The present disclosure relates generally to the use of engineered NK cells in immunotherapy for the treatment and prevention of cancer.

### BACKGROUND

An organism's immune system protects it from diseases by detecting and responding to a variety of pathogens and outside invaders, including viruses, bacteria, toxins, parasites, and wood splinters. The immune system is made up of a complex network of organs, cells and molecules. Many organisms, including humans, have two main subparts of the immune system: the innate immune system and the adaptive immune system. The innate immune system is a rapid response system which can be quickly activated to recognize and destroy pathological cells, while the adaptive immune system is a slow response system which relies on previous encounters with a specific pathogen to fight them.

Natural killer (NK) cells are granular lymphocytes and effector cells of the innate immune system. They reside in both lymphoid and nonlymphoid tissues and are different from the lymphocytes of the adaptive immune system, such as T and B cells. NK cells usually do not express CD3 or antigen-specific receptors at their cell surface. In humans, CD56 may serve as marker for NK cells. NK cells are involved in rapid response to detect and clear virus-infected cells and immune surveillance of tumor cells without the requirement of prior immunization.

Given their roles in innate immune response to tumors and viral infections, NK cells may hold promise in efforts to develop innovative therapies in fields such as antitumor immunotherapy, inflammatory and autoimmune disorders and organ transplantation.

### SUMMARY

In one aspect, a modified NK cell is provided, comprising an NK cell that expresses at least one T cell receptor, comprising at least one γ chain and at least one δ chain. In embodiments, the NK cell further comprises at least one NK cell receptor. In embodiments, the NK cell further expresses a CD3 protein. In embodiments, the at least one δ chain comprises any one or more of a δ1 chain, a δ2 chain, a δ3 chain, and a δ5 chain. In embodiments, the at least one δ chain comprises a δ2 chain. In embodiments, the at least one γ chain comprises a γ9 chain. In embodiments, the at least one T cell receptor comprises at least one Vγ9Vδ2 T cell receptor. In embodiments, the NK cell comprises an NK92 cell, a KHYG-1 cell, an NKL cell, an NKG cell, an NK-YS cell, a HANK-1 cell, or a YT cell. In embodiments, the NK cell comprises an NK92 cell. In embodiments, the NK cell comprises a primary NK cell. In other embodiments, the NK cell comprises an immortalized NK cell. In certain embodiments, the NK cells are derived from bone marrow hematopoietic stem cell precursor cells (HSCPC) or embryonic stem cells (ES cells).

In one aspect, NK cells are provided which lack substantial CD16 expression. In certain embodiments, the NK cells are selected for a lack of CD16 expression. In some embodiments, NK cells lacking CD16 expression are selected by limited dilution and expansion of monoclonal NK cell lines. CD16 protein expression can be determined by standard methods including western blot or flow cytometry using anti-CD16 antibodies. CD16 mRNA expression can be determined by standard methods including quantitative RT-PCR, digital RT-PCR or next-generation RNA sequencing (i.e., RNA-seq).

In a further aspect, CD16 expression in NK cells is antagonized. In certain embodiments, CD16 expression is antagonized with a small RNA that targets the CD16 mRNA and interferes with its expression. In some such embodiments, the small RNA is an siRNA, miRNA or shRNA.

In another aspect, CD16 expression is antagonized by inactivating the expression of one or both copies of the CD16 gene with a gene editing system. In certain such embodiments, the gene editing system includes nucleotide-guided nucleases such as, for example, a CRISPR/Cas system, a zinc-finger nuclease system or a Transcription Activator-Like Effector Nucleases (“TALENs”) system. In further embodiments, CD16 expression is antagonized by targeted protein degraders such as, for example, proteolysis targeting chimera molecules (“PROTAC”) degraders, molecular glue degraders or ubiquitin ligase modifiers.

In one aspect, NK cells are primary NK cells purified from the blood of a patient. In some such embodiments, the NK cells are purified by any method known in the art such as, for example, leukapheresis or density gradient centrifugation. In certain embodiments, purified NK cells are expanded ex vivo and transduced with one or more viral integrating or non-integrating vectors encoding at least one T cell receptor, wherein the at least one T cell receptor comprises at least one γ chain and at least one δ chain. In further such embodiments, the NK cell further comprises at least one NK cell receptor. In embodiments, purified NK cells are expanded ex vivo are transduced with a vector for expression of a CD3 protein. In embodiments, the at least one δ chain comprises any one or more of a δ1 chain, a δ2 chain, a δ3 chain, and a δ5 chain. In embodiments, the at least one δ chain comprises a δ2 chain. In embodiments, the at least one γ chain comprises a γ9 chain. In embodiments, the at least one T cell receptor comprises at least one Vγ9Vδ2 T cell receptor.

In another aspect, purified NK cells are gene edited to inactivate one or both copies of the endogenous CD16 gene(s) before being transduced with a viral vector (integrating or non-integrating) encoding at least one T cell receptor and/or at least one CD3 protein, then selected for NK cells which lack expression of CD16, and then expanded ex vivo. In certain of these embodiments, the modified NK cells are delivered back into the patient from which the NK cells were initially purified. In other embodiments, the modified NK cells are delivered into a different patient.

In other aspects, pharmaceutical compositions are provided comprising the modified NK cells described above. In certain embodiments, the pharmaceutical composition of NK92γδTCR cells, further comprising an inhibitor of CD16. In certain embodiments, the pharmaceutical composition further comprises a CD16 antagonist. The CD16 antagonist can include an siRNA, miRNA, shRNA, targeted protein degrader, small molecule inhibitor, anti-CD16 antibody or the like. In embodiments, one or more endogenous CD16 genes are inactivated or deleted from NK cells expressing the Vγ9Vδ2 T cell receptor. In certain embodiments, the antagonist of CD16 is administered separately from the pharmaceutical composition. In other embodiments, the inhibitor of CD16 is administer together with the pharmaceutical composition.

In further aspects, the pharmaceutical composition is useful for treating cancer, wherein the composition comprises a combination of: an aminobisphosphonate drug; and any modified NK cell described herein. In embodiments, the aminobisphosphonate drug comprises zoledronic acid. In embodiments, the combination comprises a fixed combination. In embodiments, the combination comprises a non-fixed combination. In embodiments, the pharmaceutical composition further comprises an inhibitor of FDPS. In embodiments, the inhibitor of FDPS comprises an siRNA, shRNA or miRNA.

In an aspect, a pharmaceutical composition for treating cancer is provided comprising a combination of: an aminobisphosphonate drug; and any modified NK cell described herein. In embodiments, the aminobisphosphonate drug comprises zoledronic acid. In embodiments, the combination comprises a fixed combination. In embodiments, the combination comprises a non-fixed combination. In embodiments, the pharmaceutical composition further comprises an inhibitor of FDPS. In embodiments, the inhibitor of FDPS comprises a shRNA or an miRNA.

In an aspect, a method of treating cancer in a subject is provided, comprising administering to the subject an effective amount of any of the modified NK cells described herein. In embodiments, the subject has at least one cancer selected from one or more of a carcinoma, a leukemia, a lymphoma, a sarcoma, a myeloma, a mesothelioma, a mixed type cancer, a glioma, a neuroblastic tumor, or mixtures thereof. In embodiments, the method further comprises administering an effective amount of an aminobisphosphonate drug to the subject. In embodiments, the aminobisphosphonate drug comprises zoledronic acid. In embodiments, the aminobisphosphonate drug is administered separately from the modified NK cell. In embodiments, the aminobisphosphonate drug is administered together with the modified NK cell. In embodiments, the method further comprises administering an effective amount of an inhibitor of FDPS. In embodiments, the inhibitor of FDPS is administered separately from the modified NK cell. In embodiments, the inhibitor of FDPS is administered together with the modified NK cell. In embodiments, the inhibitor of FDPS comprises an siRNA, shRNA or an miRNA.

### BRIEF DESCRIPTION OF THE DRAWINGS

In this disclosure:

FIG. 1 depicts a schematic of a NK92 cell that has been engineered to include a Vγ9Vδ2 T cell receptor.

FIG. 2 depicts an exemplary 3-vector lentiviral vector system in circularized form.

FIG. 3 depicts an exemplary 4-vector lentiviral vector system in circularized form.

FIG. 4 depicts lentivirus transfer plasmids encoding transgenes capable of expressing (i) CD3 proteins (gamma, delta, epsilon, and zeta), and (ii) FDPS shRNA.

FIG. 5 depicts (i) a linear vector map capable of expressing Vγ9Vδ2 T cell receptor, (ii) a linear vector map capable of expressing FDPS shRNA, and (iii) a linear vector map capable of expressing CD3.

FIG. 6A depicts flow cytometry data in which, relative to the left panel, the right panel shows NK92 cells that are enriched for the γδ T cell receptor and the CD3 protein.

FIG. 6B depicts flow cytometry data showing that only Vγ9Vδ2 T cell receptor positive NK92 cells responded to zoledronic acid-treated C8166 cells.

FIG. 6C depicts a graph showing that NK92 cells engineered with a γδ T cell receptor were able to lyse C8166 cells after treatment with zoledronic acid.

FIG. 7 depicts a schematic of limited dilution cloning of transduced NK92 cells in which individual cells that were positive for transduction were chosen for expansion.

FIG. 8 depicts screening of several sublines of transduced cells using a CD107a degranulation assay. The results show that NK92-γδTCR-5 subline was the most responsive subline when exposed to C8166 cells treated with zoledronic acid.

FIG. 9 depicts screening of several sublines of transduced cells using a TNFα expression assay. The results demonstrate that NK92-γδTCR-5 subline was the most responsive subline when exposed to C8166 cells and treated with zoledronic acid.

FIG. 10 depicts a cytotoxicity assay in which a B lymphoblastoid cell line (Daudi cells) was exposed to the NK92-γδTCR-5 (NK92-gdTCR) subline. The NK92-γδTCR-5 subline was capable of killing the Daudi cells; killing was increased after treatment with zoledronic acid.

FIG. 11 depicts screening of NK92 cells v. the NK92-γδTCR-5 subline (labelled “NK92-γδTCR” in FIG. 11) using a degranulation assay, with Daudi cell targets. Relative to the NK92 cells, the NK92-γδTCR-5 subline showed a potent response.

FIG. 12 depicts screening of NK92 cells v. the NK92-γδTCR-5 subline (labelled “NK92-γδTCR” in FIG. 12) using a TNFα expression assay, with Daudi cell targets. Relative to the NK92 cells, the NK92-γδTCR-5 subline showed a potent response.

FIG. 13 depicts a cytotoxicity assay in which the TU167 tumor cell targets were exposed to the NK92-γδTCR-5 subline (labelled “NK92-gdTCR” in FIG. 13). Potent killing of cells was observed when the TU167 cells were exposed to the NK92-γδTCR-5 subline and treated with zoledronic acid. The strongest killing of cells was observed when TU167 cells were (i) transduced with a vector that expressed an miRNA inhibitor of farnesyl diphosphate synthase (LV-FDPS) and (ii) treated with zoledronic acid.

FIG. 14 depicts a cytotoxicity assay in which Huh7 tumor cell targets were exposed to the NK92-γδTCR-5 subline (labelled “NK92-gdTCR” in FIG. 14). Potent killing was observed when the Huh7 cells were exposed to the NK92-γδTCR-5 subline and treated with zoledronic acid. Potent killing was also observed when the Huh7 cells were exposed to the NK92-γδTCR-5 subline and treated with zoledronic acid along with a vector that expressed an inhibitor of farnesyl diphosphate synthase (LV-FDPS).

FIG. 15 depicts results from a screen of cloned NK92-γδTCR cell lines with NK92 (control) and NK92-γδTCR-5 added. The cloned cell line response to tumor cells (“Daudi) or tumor cells plus zoledronic acid (“Daudi+Zol”) are indicated by the percentage of cells staining positive for the presence of Vδ2 and CD107a. “Med” here (and throughout the figures) stands for a negative control where cells were exposed to growth medium only.

FIG. 16 depicts the results of screening cloned Vγ2Vδ2TCR/CD3 engineered NK92 (NK92γδTCR) cells by the response to C8166 cells. The selected NK92 cell clones that express Vγ9Vδ2 T Cell Receptor were treated with medium or C8166 cells for 4 hours. CD107a was detected as a marker of cell response.

FIG. 17 depicts the results of screening cloned Vγ2Vδ2TCR/CD3 engineered NK92 (NK92γδTCR) cells by the response to different cell lines. The selected NK92 cell clones that express Vγ9Vδ2 T Cell Receptor were treated with medium or different cell lines for 4 hours. CD107a was detected as a marker of cell response.

FIG. 18 depicts the results of screening cloned Vγ2Vδ2TCR/CD3 engineered NK92 (NK92γδTCR) cells by the response to Huh7 or zoledronic acid treated Huh7 cells. The selected NK92 cell clones that express Vγ9Vδ2 T Cell Receptor were treated with medium or C8166 cells for 4 hours. CD107a was detected as a marker of cell response.

FIG. 19 depicts results comparing the response of NK92γδTCR cell clones S76 and S77 to multiple cancer cell lines. The NK92γδTCR cell clones S76 and S77 were treated with medium or different cell lines for 4 hours. CD107a was detected as a marker of cell response.

FIG. 20 depicts the results of NK92γδTCR cell clone S76 to multiple cancer cell lines treated with medium alone or with 1 μM of zoledronic acid. CD107a was detected as a marker of cell response.

FIG. 21 depicts the results of Mitomycin C (“MMC”) treatment on the response of NK92γδTCR cell clone S76 to Daudi cells. NK92γδTCR cell clone S76 was treated with MMC at various concentration for 1 or 2 hours. After MMC treatment, S76 was treated with Daudi cells at 2:1 ratio for 4 hours. CD107a was detected as a marker of cell response.

FIG. 22 depicts the results of evaluating the cytotoxicity of NK92γδTCR-S76 or NK92γδTCR-5 against C8166 cells. The cytotoxicity of NK92γδTCR-S76 or NK92γδTCR-5 against C8166 or zoledronic acid-treated C8166 cells was evaluated at several effector to target (E:T) ratios with triplicate wells for each condition.

FIGS. 23A-23B depicts the results of cytotoxicity and degranulation assays of NK92γδTCR-S76 or NK92γδTCR-5 against SNU447 cells. FIG. 23A depicts the cytotoxicity of NK92γδTCR-S76 or NK92γδTCR-5 against SNU447 or zoledronic acid-treated SNU447 cells evaluated at several effector to target (E:T) ratios with triplicate wells for each condition. FIG. 23B depicts NK92γδTCR-S76 or NK92γδTCR-5 treated with medium, SNU447 or zoledronic acid-treated SNU447 cells for 4 hours. CD107a was detected as a marker of cell response.

FIGS. 24A-24B depicts cytotoxicity and degranulation assays of NK92γδTCR-S76 or NK92γδTCR-5 against A549 cells. FIG. 24A depicts the cytotoxicity of NK92γδTCR-S76 or NK92γδTCR-5 against A549 or zoledronic acid-treated A549 cells evaluated at several effector to target (E:T) ratios with triplicate wells for each condition. FIG. 24B depicts NK92γδTCR-S76 or NK92γδTCR-5 were treated with medium, A549 or zoledronic acid-treated A549 cells for 4 hours. CD107a was detected as a marker of cell response.

FIGS. 25A-25B depicts cytotoxicity and degranulation assays of NK92γδTCR-S76 against PC3 cells. FIG. 25A depicts the cytotoxicity of NK92γδTCR-S76 against PC3 or zoledronic acid-treated PC3 cells evaluated at several effector to target (E:T) ratios with triplicate wells for each condition. FIG. 25B depicts NK92γδTCR-S76 were treated with medium, PC3 or zoledronic acid-treated PC3 cells for 4 hours. CD107a was detected as a marker of cell response.

FIG. 26 depicts the effect of NKp44 antibodies on the response of NK92-gdTCR-S76 to Daudi cells. NK92γδTCR cell clone S76 was treated with blocking or agonist anti-NKp44 antibodies for 1 hour. After treatment, S76 was treated with Daudi cells at 1:1 ratio for 4 hours. CD107a was detected as a marker of cell response.

### DETAILED DESCRIPTION

### Overview

The present disclosure relates to NK cells that have been modified with T cell receptors and methods of using the same to treat various cancers. In embodiments, the T cell receptors are γδ T cell receptors. In embodiments, the cancer that is treated is any one or more of a carcinoma, a leukemia, a lymphoma, a sarcoma, a myeloma, a mesothelioma, a mixed type, a glioma, a neuroblastic tumor, or mixtures thereof.

### Definitions and Interpretation

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. Generally, nomenclature used in connection with, and techniques of, cell and tissue culture, molecular biology, immunology, microbiology, genetics and protein and nucleic acid chemistry and hybridization described herein are those well-known and commonly used in the art. The methods and techniques of the present disclosure are generally performed according to conventional methods well-known in the art and as described in various general and more specific references that are cited and discussed throughout the present specification unless otherwise indicated. See, e.g.: Sambrook J. & Russell D. Molecular Cloning: A Laboratory Manual, 3rd ed., Cold Spring Harbor Laboratory Press, Cold Spring Harbor, N.Y. (2000); Ausubel et al., Short Protocols in Molecular Biology: A Compendium of Methods from Current Protocols in Molecular Biology, Wiley, John & Sons, Inc. (2002); Harlow and Lane Using Antibodies: A Laboratory Manual; Cold Spring Harbor Laboratory Press, Cold Spring Harbor, N.Y. (1998); and Coligan et al., Short Protocols in Protein Science, Wiley, John & Sons, Inc. (2003). Any enzymatic reactions or purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art.

As used herein, the term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

As used herein, the terms “administration of” or “administering” an active agent means providing an active agent to the subject in need of treatment in a form that can be introduced into that individual's body in a therapeutically useful form and therapeutically effective amount.

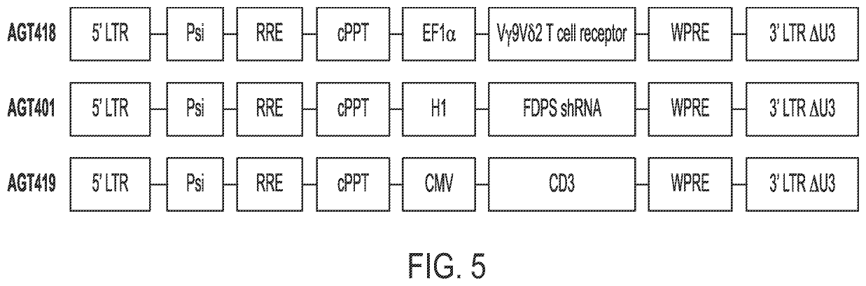

As used herein, the terms “AGT418”, “AGT401”, and “AGT419” refer to vectors as illustrated in FIG. 5.

As used herein, the term “aminobisphosphonate” refers to any amino derivative of a bisphosphonate. A bisphosphonate is a chemical that contains two (2) phosphonate groups covalently linked to a carbon.

Throughout this specification and claims, the word “comprise,” or variations such as “comprises” or “comprising,” will be understood to imply the inclusion of a stated integer or group of integers but not the exclusion of any other integer or group of integers. Further, as used herein, the term “includes” means includes without limitation. The terms, “expression,” “expressed,” or “encodes” refer to the process by which polynucleotides are transcribed into mRNA and/or the process by which the transcribed mRNA is subsequently being translated into peptides, polypeptides, or proteins. Expression may include splicing of the mRNA in a eukaryotic cell or other forms of post-transcriptional modification or post-translational modification.

As used herein, the term “farnesyl diphosphate synthase” may also be referred to herein as FDPS, and may also be referred to herein as farnesyl pyrophosphate synthase or FPPS.

As used herein, the term “gamma delta T cell receptor” refers to any T cell receptor that contains at least one gamma chain and at least one delta chain. A gamma delta T cell receptor may also be referred to herein as a ye, T cell receptor, as a GD T cell receptor, or as a gd T cell receptor.

The term “in vivo” refers to processes that occur in a living organism. The term “ex vivo” refers to processes that occur outside of a living organism. For example, in vivo treatment refers to treatment that occurs within a patient's body, while ex vivo treatment is one that occurs outside of a patient's body, but still uses or accesses or interacts with tissues from that patient. Thereafter, an ex vivo treatment step may include a subsequent in vivo treatment step.

The term “miRNA” refers to a microRNA, and also may be referred to herein as “miR”. The term “microRNA cluster” refers to at least two microRNAs that are situated on a vector in close proximity to each other and which are co-expressed together.

The term “Natural Killer cell” (also known as “NK cell”) refers to a type of lymphocyte that functions in the innate immune system. The term encompasses any Natural Killer cell that is naturally occurring. The term also encompasses any synthetic Natural Killer cell, such as a Natural Killer cell derived from a cell line.

The term “packaging cell line” refers to any cell line that can be used to express a lentiviral particle.

The term “percent identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of ordinary skill in the art) or by visual inspection. Depending on the application, the “percent identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information website.

The percent identity between two nucleotide sequences can be determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences of the present disclosure can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, word length=12 to obtain nucleotide sequences homologous to the nucleic acid molecules. BLAST protein searches can be performed with the XBLAST program, score=50, word length=3 to obtain amino acid sequences homologous to the protein molecules. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

As used herein, the term “SEQ ID NO” is synonymous with the term “Sequence ID No.”

As used herein, “small RNA” refers to non-coding RNA that are generally less than about 200 nucleotides or less in length and possess a silencing or interference function. In other embodiments, the small RNA is about 175 nucleotides or less, about 150 nucleotides or less, about 125 nucleotides or less, about 100 nucleotides or less, or about 75 nucleotides or less in length. Such RNAs include microRNA (miRNA), small interfering RNA (siRNA), double stranded RNA (dsRNA), and short hairpin RNA (shRNA). “Small RNA” of the disclosure should be capable of inhibiting or knocking-down gene expression of a target gene, for example through pathways that result in the destruction of the target gene mRNA.

As used herein, the term “subject” includes a human patient but also includes other mammals. The terms “subject,” “individual,” “host,” and “patient” may be used interchangeably herein.

The term “therapeutically effective amount” refers to a sufficient quantity of the active agents, in a suitable composition, and in a suitable dosage form to treat or prevent the symptoms, progression, or onset of the complications seen in patients suffering from a given ailment, injury, disease, or condition. The therapeutically effective amount will vary depending on the state of the patient's condition or its severity, and the age, weight, etc., of the subject to be treated. A therapeutically effective amount can vary, depending on any of a number of factors, including, e.g., the route of administration, the condition of the subject, as well as other factors understood by those in the art.

The term “treatment” or “treating” generally refers to an intervention in an attempt to alter the natural course of the subject being treated, and can be performed either for prophylaxis or during the course of clinical pathology. Desirable effects include, but are not limited to, preventing occurrence or recurrence of disease, alleviating symptoms, suppressing, diminishing or inhibiting any direct or indirect pathological consequences of the disease, ameliorating or palliating the disease state, and causing remission or improved prognosis.

As used herein, the term “Vγ9Vδ2” refers to a type of γδ receptor that is capable of recognizing conformational changes in butyrophilins. The term Vγ9Vδ2 is synonymous with Vg9Vd2. Vγ9V62 is a shorthand for Vγ9JPV62 (Vg9JPVd2). The V refers to a region, and can include any one or more of the V2 region, the V3 region, the V4 region, the V8 region, and the V9 region. The γ refers to at least one γ chain, and can include any one or more of the constant regions C1 and C2. The J refers to at least one J region and can include any one or more of the JP1 region, the JP region, the J1 region, the JP2 region, and the J2 region.

### Description of Aspects and Embodiments of the Disclosure

In an aspect, a modified NK cell is provided, comprising an NK cell that expresses at least one T cell receptor that comprises at least one γ chain and at least one δ chain. In embodiments, the NK cell further comprises at least one NK cell receptor.

In embodiments, the NK cell further expresses a CD3 protein complex. In embodiments, the CD3 protein complex comprises any one or more of CD3 proteins gamma, delta, epsilon, and zeta. In embodiments, the NK cell is modified such that it contains each of the CD3 proteins-CD3 protein gamma, CD3 protein delta, CD3 protein epsilon, and CD3 protein zeta. In embodiments the vector used to express the CD3 proteins in the NK cell is AGT419 (see FIG. 5).

In embodiments, the vector that expresses the CD3 proteins comprises at least 80% sequence identity with SEQ ID NO: 9, for example, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with SEQ ID NO: 9. In embodiments, the vector that expresses the CD3 proteins comprises SEQ ID NO: 9.

In embodiments, the at least one δ chain comprises any one or more of a 61 chain, a 62 chain, a 63 chain, and a 65 chain. In embodiments, the at least one δ chain comprises a 62 chain. In embodiments, the at least one γ chain comprises a γ9 chain.

In embodiments, the at least one T cell receptor comprises at least one Vγ9Vδ2 T cell receptor. In embodiments, the Vγ9Vδ2 T cell receptor is naturally occurring. In embodiments, the Vγ9Vδ2 T cell receptor is synthetic. In embodiments, the vector used to express the Vγ9Vδ2 T cell receptor in the NK cell is AGT418 (see FIG. 5).

In embodiments, the vector that expresses the Vγ9Vδ2 T cell receptor comprises at least 80% sequence identity with SEQ ID NO: 5, for example, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with SEQ ID NO: 5. In embodiments, the vector that expresses the Vγ9Vδ2 T cell receptor comprises SEQ ID NO: 5.

In embodiments, the NK cell comprises an NK92 cell, a KHYG-1 cell, an NKL cell, an NKG cell, an NK-YS cell, a HANK-1 cell, or a YT cell. In embodiments, the NK cell comprises an NK92 cell. In embodiments, the NK cell comprises an NK cell derived from any known NK cell line.

In embodiments, the NK cell comprises a primary NK cell.

In an aspect, a pharmaceutical composition for treating cancer is provided, comprising a combination of: an aminobisphosphonate drug; and any modified NK cell described herein. In embodiments, the aminobisphosphonate drug comprises zoledronic acid. In embodiments, the combination comprises a fixed combination. In embodiments, the combination comprises a non-fixed combination.

In embodiments, the pharmaceutical composition, further comprises an inhibitor of FDPS. In embodiments, the inhibitor of FDPS is a small RNA. In embodiments, the small RNA comprises an siRNA, shRNA or an miRNA.

In embodiments, the shRNA comprises at least 80% sequence identity with any of SEQ ID NOs: 50, 51, 52, or 53, for example, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with any of SEQ ID NOs: 50, 51, 52, or 53. In embodiments, the shRNA comprises any of SEQ ID NOs: 50, 51, 52, or 53.

In embodiments, the microRNA comprises at least 80% sequence identity with any of SEQ ID NOs: 64, 65, or 66, for example, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with any of SEQ ID NOs: 64, 65, or 66. In embodiments, the microRNA comprises any of SEQ ID NOs: 64, 65, or 66.

In embodiments, a vector is used to express the small RNA. In embodiments, the vector that is used is AGT401 (see FIG. 5). In embodiments, the shRNA cassette of AGT401 is substituted for an siRNA or miRNA. In embodiments, the H1 promoter of transcription of AGT401 is substituted for an EF1α promoter of transcription.

In an aspect, a pharmaceutical composition for treating cancer is provided, comprising a combination of: a competitive inhibitor of the enzyme farnesyl diphosphate synthase; and any modified NK cell described herein. In embodiments, the competitive inhibitor of the enzyme farnesyl diphosphate synthase comprises zoledronic acid, alendronate, pamidronate, risedronate, ibandronate, or etidronate.

In an aspect, a pharmaceutical composition for treating cancer is provided, comprising a combination of: a bisphosphonate or an aminobisphosphonate compound; and any modified NK cell described herein.

In an aspect, a method of treating cancer in a subject is provided, comprising administering to the subject an effective amount of any of the modified NK cells described herein.

In embodiments, the NK cells express Vγ9Vδ2 receptors. In embodiments, the Vγ9Vδ2 receptors recognize and complex with butyrophilins. In embodiments, the butyrophilins have undergone a conformational change. In embodiments, the butyrophilins derive from cancer cells. In embodiments, the butyrophilins undergo a conformational change in the tumor cells as a result of high levels of isopentenyl pyrophosphate (IPP).

In embodiments, the subject has at least one cancer selected from one or more of a carcinoma, a leukemia, a lymphoma, a sarcoma, a myeloma, a mesothelioma, a mixed type, a glioma, a neuroblastic tumor, or mixtures thereof. In embodiments, the subject has any cancer that is disclosed herein.

In embodiments, the method further comprises administering an effective amount of an aminobisphosphonate drug to the subject. In embodiments, the aminobisphosphonate drug comprises zoledronic acid.

In embodiments, the aminobisphosphonate drug is administered separately from the modified NK cell. In embodiments, the aminobisphosphonate drug is administered together with the modified NK cell.

In embodiments, the method further comprises administering an effective amount of an inhibitor of FDPS. In embodiments, the inhibitor of FDPS is administered separately with the modified NK cell. In embodiments, the inhibitor of FDPS is administered together with the modified NK cell. In embodiments, the inhibitor of FDPS comprises a shRNA, a microRNA, or an siRNA. In embodiments, the inhibitor of FDPS is any small RNA.

In embodiments, the shRNA comprises at least 80% sequence identity with any of SEQ ID NOs: 50, 51, 52, or 53, for example, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with any of SEQ ID NOs: 50, 51, 52, or 53. In embodiments, the shRNA comprises any of SEQ ID NOs: 50, 51, 52, or 53.

In embodiments, the microRNA comprises at least 80% sequence identity with any of SEQ ID NOs: 64, 65, or 66, for example, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with any of SEQ ID NOs: 64, 65, or 66. In embodiments, the microRNA comprises any of SEQ ID NOs: 64, 65, or 66.

In an aspect, a method of treating cancer is provided comprising administering to the subject an effective amount of any of the pharmaceutical compositions described herein. In embodiments, the subject has at least one cancer selected from one or more of a carcinoma, a leukemia, a lymphoma, a sarcoma, a myeloma, a mesothelioma, a mixed type, a glioma, a neuroblastic tumor, or mixtures thereof. In embodiments, the subject has any cancer described herein.

In embodiments, the aminobisphosphonate drug comprises zoledronic acid. In embodiments, the method further comprises administering an effective amount of an inhibitor of FDPS. In embodiments, the inhibitor of FDPS is administered separately with the pharmaceutical composition. In embodiments, the inhibitor of FDPS is administer together with the pharmaceutical composition. In embodiments, the inhibitor of FDPS is a shRNA or a microRNA.

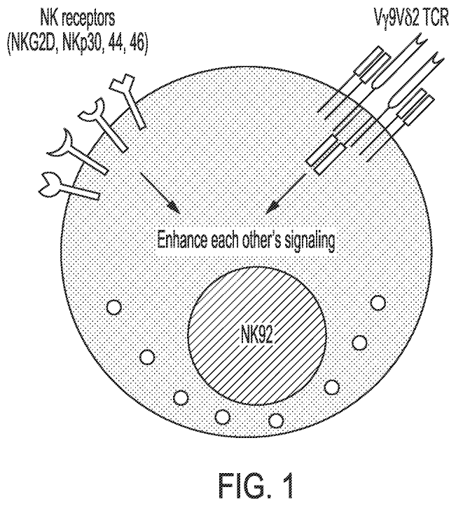

Turning now to FIG. 1, which depicts an NK cell modified with a Vγ9Vδ2 T cell receptor. The combination of the naturally occurring NK receptors, such as, for example, NKG2d, NKp30, 44, and 46, amongst others, with the exogenous Vγ9Vδ2 T cell receptor causes an enhancement of intracellular signaling. In embodiments, this enhanced signaling creates an effector cell that is effective in killing tumor and cancer cells.

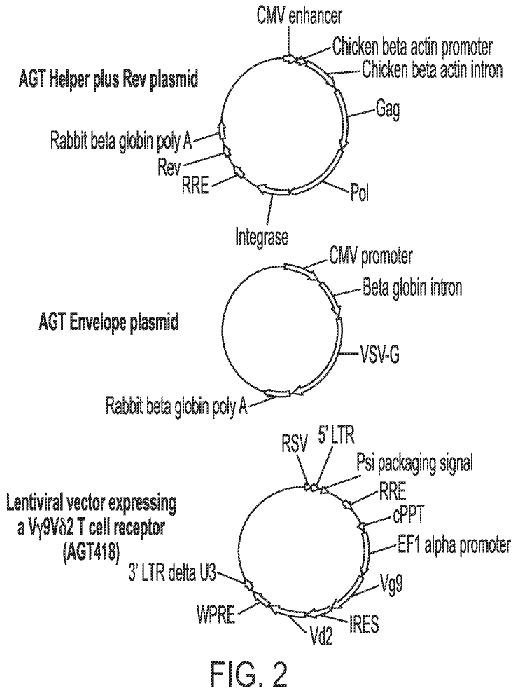

Turning now to FIG. 2, which depicts a three plasmid system for generating lentivirus vector AGT418 (see FIG. 5), a vector that can be used to modify NK92 cells to express the Vγ9Vδ2 T cell receptor. Individual plasmids are: AGT Helper plus Rev—encodes structural proteins for vector packaging; AGT Rev plasmid—encodes the Rev regulatory element; AGT Envelope plasmid encodes the VSV-G envelope glycoprotein; and Lentivirus vector transfer plasmid encoding a transgene capable of expressing a Vγ9Vδ2 T cell receptor.

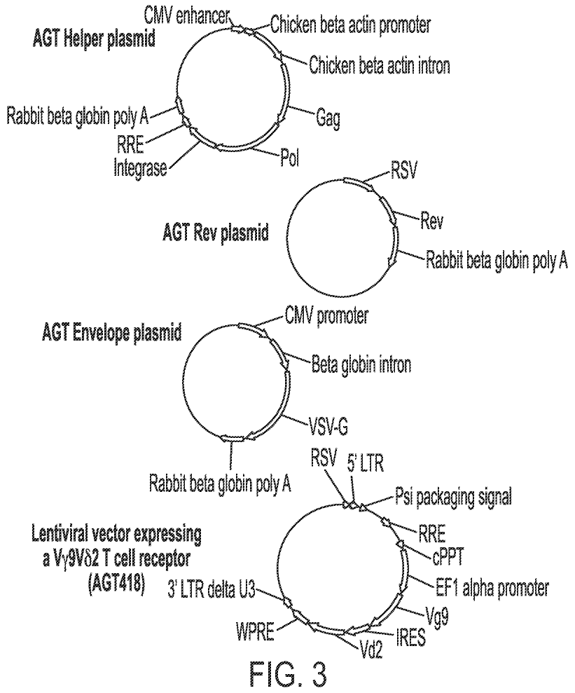

Turning now to FIG. 3, which depicts a four plasmid system for generating lentivirus vector AGT418 (see FIG. 5), a vector that can be used to modify NK92 cells to express the Vγ9Vδ2 T cell receptor. Individual plasmids are: AGT Helper plasmid—encodes structural and regulatory proteins for vector packaging; AGT Envelope plasmid—encodes the VSV-G envelope glycoprotein; and Lentivirus vector transfer plasmid encoding a transgene capable of expressing a Vγ9Vδ2 T cell receptor.

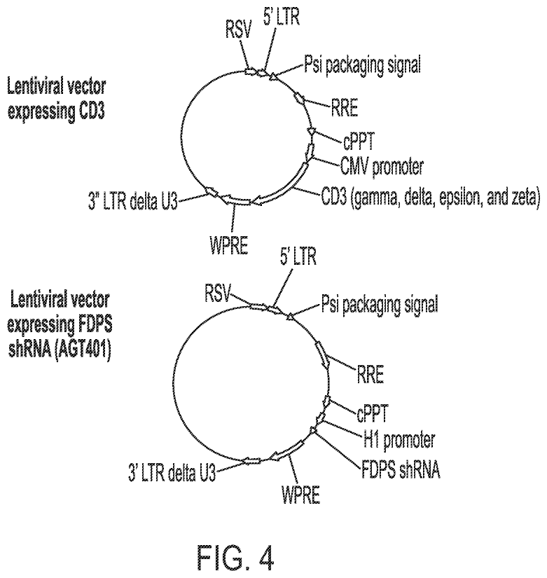

Turning now to FIG. 4, which depicts (i) a lentivirus vector transfer plasmid encoding a transgene capable of expressing CD3 proteins (gamma, delta, epsilon and zeta), and (ii) a lentivirus vector transfer plasmid encoding a transgene capable of expressing microRNA for downregulating expression of farnesyl diphosphate synthase (AGT401; see FIG. 5). In embodiments, as described herein, a vector carrying CD3 genes was used to modify NK92 cells (CD3 proteins are not expressed in NK92 but are required for T cell receptor function). In embodiments, a vector (AGT401; see FIG. 5) was used to modify tumor cells for characterization of the NK92-Vγ9Vδ2 T cell receptor effector cell line. These plasmids can be used in concert with AGT helper plus Rev plasmid and AGT envelope plasmid in a 3 plasmid packaging system (see FIG. 2), or AGT helper plasmid, AGT Rev plasmid, and AGT envelope plasmid in a four plasmid system (see FIG. 3), to manufacture lentivirus vector particles.

### Natural Killer Cells

Natural Killer cells (also known as NK cells) are a type of lymphocyte that function in the innate immune system. NK cells are capable of recognizing MHC (Major Histocompatibility Complex) on infected cell surfaces independently of the any specific epitope or neoepitope peptide being bound to the MHC molecule; however, they are also capable of recognizing stressed cells without the need of the MHC. Thus, unlike T cells expressing an alpha/beta type T cell receptor viz., CD4 and CD8 T cells, NK cells are capable of recognizing and destroying harmful cells with little or no cell surface MHC.

NK cell surface receptors can trigger cell-mediated cytotoxicity and cytokine production. NK cells can produce cytolytic granules inside their cytoplasm which can release cytotoxic molecules, such as perforin and granzymes, to induce lysis of target cells. NK cells can also secrete IFN-γ and TNFα cytokines which can lead to phagocytosis and lysis of target cells. Furthermore, NK cells can eliminate a target cell, such as a tumor cell, through antibody-dependent cell cytotoxicity if the target cell is tagged with an antibody that recognizes a pathogen's antigen. Besides their ability to lyse target cells, NK cells are also involved in promoting inflammatory immune responses through cytokine and chemokine release.

### Gamma/Delta T Cells and Gamma/Delta T Cell Receptors

Human gamma/delta T cells are grouped and characterized according to the specific delta chain used in each cell. Most common is the delta2 chain (written Vd2) that is common in blood and secondary lymphoid tissues. Second comes the delta1 cells (Vd1) that appear in blood and mucosal epithelia. Minor populations of Vd3 and Vd5 cells are widely distributed and have specialized functions. Most importantly, the gamma/delta TCR frequently react to the presence of non-peptide antigens and are mostly independent of MHC proteins. While each case is unique, gamma/delta T cells may be highly similar even among unrelated individuals in a population because they do not require MHC function and there is not a strong driver of T cell receptor repertoire diversity. Gamma/delta T cell recognition of tumor for example, is restricted to cells of the same species but the concept of allogenicity or participating in graft versus host disease do not apply to gamma/delta T cells.

In the specific case of Vg9Vd2 T cells, the recognition mechanisms are well studied. It is known that tumor cells have a higher proliferation index compared to normal cells and often express a common protein from the family of butyrophilins. Because of high proliferation and the consequent high demand for membrane synthesis, tumor cells overproduce intermediate molecules in the cholesterol biosynthesis pathway including isopentenyl pyrophosphate (IPP), a 5 carbon isoprenoid. Abnormally high cytoplasmic levels of IPP bind to and cause a conformational change in cell surface butyrophilin. The Vg9Vd2 TCR evolved to recognize this conformational change and interpret it as a danger signal. Formation of the Vg9Vd2 TCR:butyrophilin complex activates gamma delta T cells causing proliferation and differentiation. Activated cells produce cytokines and often become potently cytotoxic as part of the natural mechanism for tumor surveillance. Since butyrophilin genes are nearly identical in all people, the Vg9Vd2 T cells from one person are equally efficient for recognizing their own tumor cell or a tumor cell from someone else. Since butyrophilin is expressed on most tumor cells and proliferation (higher IPP) is a common feature of cancer, Vg9Vd2 T cells can be exploited for cancer therapy irrespective of MHC differences and largely irrespective of the tumor type. The Vg9Vd2 T cells constitute something that is nearly universal for tumor recognition and destruction.

Engineered NK Cells that Express Naturally Occurring Gamma/Delta TCR

NK cells can be engineered to express a naturally occurring gamma/delta TCR. In embodiments, the engineered cells demonstrate strong activation and potent tumor cell killing potential. In embodiments, a purpose of the engineered NK cells is to produce a product that will substitute for natural gamma delta T cells in cancer therapy, become an inexhaustible source of tumor effector cells, and function equally among all persons with cancer.

An important barrier to maximum potency of genetically engineered NK cells is heterogeneity within the population of transduced cells. Heterogeneity causes the population of cells to function, for tumor cell killing, at a potency far below the levels seen for individual cells within the population. Heterogeneity among transduced cells also impacts feasibility of product manufacturing, as each transduction is expected to generate a unique, heterogeneous population, and complicates the analysis of product safety regarding suitability for clinical use.

In embodiments, mitigating the heterogeneity problem involves generating cloned cell lines starting from the heterogeneous population of transduced cells. Because cloned cell lines are generated from individual cells, a high level of homogeneity is maintained. Further, cloned cell lines can be characterized to determine the level of ye, TCR expression and the functional responses to tumor cells. In embodiments, characterization of cloned cell lines allows selection of individual cloned cell lines best suited for cancer therapy.

### CD16

Surprisingly, the inventors have discovered that NK cells which lack substantial CD16 expression show enhanced expression of the Vγ9Vδ2 T cell receptor and/or enhanced recognition and lysis of cancer cells.

In one aspect, NK cells are provided which lack substantial CD16 expression. In certain embodiments, the NK cells are selected for a lack of CD16 expression. In some embodiments, NK cells lacking CD16 expression are selected by limited dilution and expansion of monoclonal NK cell lines. CD16 protein expression can be determined by standard methods including western blot or flow cytometry using anti-CD16 antibodies. CD16 mRNA expression can be determined by standard methods including quantitative RT-PCR, digital RT-PCR or next-generation RNA sequencing (i.e., RNA-seq).

In a further aspect, CD16 expression in NK cells is antagonized. In certain embodiments, CD16 expression is antagonized with a small RNA that targets the CD16 mRNA and interferes with its expression. In some such embodiments, the small RNA is an siRNA, miRNA or shRNA.

In another aspect, CD16 expression is antagonized by inactivating the expression of one or both copies of the CD16 with gene editing. In certain such embodiments, the gene editing system includes nucleotide-guided nucleases such as, for example, a CRISPR/Cas system, zinc-finger nucleases or Transcription Activator-Like Effector Nucleases (“TALENs”) system. In further embodiments, CD16 is antagonized by targeted protein degraders such as, for example, proteolysis targeting chimera molecules (“PROTAC”) degraders, molecular glue degraders or ubiquitin ligase modifiers.

In one aspect, NK cells are primary NK cells purified from the blood of a patient. In some such embodiments, the NK cells are purified by any method known in the art such as, for example, leukapheresis or density gradient centrifugation. In certain embodiments, purified NK cells are expanded ex vivo and transduced with one or more viral integrating or non-integrating vectors encoding at least one T cell receptor, wherein the at least one T cell receptor comprises at least one γ chain and at least one δ chain. In further such embodiments, the NK cell further comprises at least one NK cell receptor. In embodiments, purified NK cells are expanded ex vivo are transduced with a vector for expression of a CD3 protein. In embodiments, the at least one δ chain comprises any one or more of a 61 chain, a 62 chain, a 63 chain, and a 65 chain. In embodiments, the at least one δ chain comprises a 62 chain. In embodiments, the at least one γ chain comprises a γ9 chain. In embodiments, the at least one T cell receptor comprises at least one Vγ9Vδ2 T cell receptor.

In another aspect, purified NK cells are gene edited to inactivate one or both copies of the endogenous CD16 gene(s) before being transduced with a viral vector (integrating or non-integrating) encoding at least one T cell receptor and/or at least one CD3 protein, then selected for NK cells which lack expression of CD16, and then expanded ex vivo. In certain of these embodiments, the modified NK cells are delivered back into the patient from which the NK cells were initially purified. In other embodiments, the modified NK cells are delivered into a different patient.

In other aspects, pharmaceutical compositions are provided comprising the modified NK cells described above. In certain embodiments, the pharmaceutical composition further comprises a CD16 antagonist. The CD16 antagonist can include an siRNA, miRNA, shRNA, targeted protein degrader, small molecule inhibitor, anti-CD16 antibody or the like. In certain embodiments, the antagonist of CD16 is administered separately with the pharmaceutical composition. In other embodiments, the inhibitor of CD16 is administer together with the pharmaceutical composition.

In another aspect, methods are provided for decreasing the levels of CD16 in NK cells to facilitate gd TCR expression and consequent recognition and killing of tumor cells as a therapy for liquid and solid tumors. The method involves bioinformatic analysis of the CD16 mRNA coding sequence using freely available, standard bioanalytical tools to define optimum target sites for inhibitory RNA. A collection of likely target sites is compiled numbering between 5 and 20 for the CD16 mRNA sequence. Short interfering RNA (siRNA) molecules with sequences complementary to these targets (guide sequences) can be synthesized or obtained from commercial suppliers.

The siRNA guide sequences can be delivered to cells via direct RNA transfection, in the form of plasmid constructs or encoded in suitable integrating or non-integrating viral vectors. The siRNA may be combined into the backbone of short-hairpin RNA (shRNA) or micro-RNA (miRNA). Suitable shRNA or miRNA backbones are known in the art and the processes for constructing suitable expression clones including siRNA, shRNA or miRNA plus promoter/enhancer regions and transcription terminators if necessary, are known to persons of ordinary skill in the art of molecular biology. The shRNA and miRNA constructs are delivered to cells or tissues in RNA or DNA form and are processed by cellular enzymes to release the siRNA sequence. The siRNA anneals to CD16 mRNA at the target sites and promotes RNA cleavage, destabilization and degradation. The effect of individual siRNAs can be compared in cell transfection or transduction assays to determine the extent of reduction for CD16 mRNA, and CD16 protein levels on the cell surface. Further studies can be conducted using standard approaches, to evaluate safety and specificity of the most potent siRNA and a final siRNA or multiple siRNAs are selected for NK cell modification.

NK cells, including primary and immortalized cells, or bone marrow hematopoietic stem cell precursor cells (HSCPC), or embryonic stem cells (ES cells) can be used. These cells can be treated with siRNA, shRNA, or miRNA containing potent, specific and safe guide sequences to reduce CD16 mRNA and protein levels. The siRNA, shRNA or miRNA can be delivered in RNA form by transfection using known methods, or delivered by transfection of DNA plasmids, or delivered by transduction using a suitable integrating or non-integrating viral vector.

NK cells, cell lines, HSCPC or ES cells treated stably or transiently with CD16-specific siRNA can be treated with genes encoding the CD3 protein complex using DNA plasmid transfection, or transduction with integrating or non-integrating viral vectors. Treated cells can be tested for the expression of CD3 proteins and for cell surface expression of the gamma delta T cell receptor. Cells positive for CD3 and gd TCR are selected, cloned and enriched from the starting culture, cultured to increase cell numbers, and tested for activation by exposure to tumor cells and for their capacity to lyse tumor cells through cellular cytotoxicity.

These methods may be used to generate NK cells or immortalized cell lines expressing the gd TCR and capable of tumor recognition and killing. Modified HSCPC or ES cells may be implanted in subjects suffering from cancer where the modified NK cells are therapeutic for their cancer. Additionally, persons at high risk for cancer due to environmental exposure or heredity may be treated with modified NK, HSCPC or ES cells as a prophylactic therapy to reduce the chances for fatal cancer onset.

In another aspect, methods are provided for editing the CD16 gene in NK cells to prevent expression of the protein and thus, facilitate gd TCR expression and consequent recognition and killing of tumor cells as a therapy for liquid and solid tumors. The method involves bioinformatic analysis of the CD16 gene sequence using freely available, standard bioanalytical tools to define optimum target sites for guide RNA, for zinc-finger nuclease recognition, or for TALEN recognition or as a target for other known gene editing systems. A collection of likely guide sequences is compiled numbering between 5 and 20 for the CD16 gene. Guide RNA molecules or appropriately targeted Zinc-finger, or TALEN nuclease systems can be synthesized or obtained from commercial suppliers.

The editing constructs can be delivered to cells via direct RNA transfection, in the form of plasmid constructs or encoded in suitable integrating or non-integrating viral vectors. DNA editing enzymes such as CRISPR/Cas9 and its derivatives and similar systems, Zinc-finger nucleases and TALEN molecules may be delivered by transfection as purified RNA, or by transfection using plasmid DNA, or by engineering suitable integrating or non-integrating viral vectors capable of delivering the editing machinery by transduction. Suitable guide RNA and editing machinery are known in the art and the processes for constructing suitable expression clones including guide RNA and enzyme coding sequences plus promoter/enhancer regions and transcription terminators if necessary, are known to persons or ordinary skill in the art of molecular biology. The editing constructs are delivered to cells or tissues in RNA or DNA form. The guide RNA or engineered site recognition elements in Zinc-finger nucleases and TALEN molecules, recognize target sequences in the CD16 gene and introduce mutations and/or deletions to inactivate gene expression. Individual gene editing constructs are compared in cell transfection or transduction assays to determine the efficiency of biallelic CD16 gene modification. Further studies using standard assays can be used to evaluate the safety and specificity of the most efficient gene editing approaches for editing NK, cell lines, HSCPC or ES cells.

In another aspect, NK cells, NK cell lines, HSCPC or ES cells with biallelic modification of the CD16 alleles are transduced with appropriate integrating or non-integrating vectors or transfected with RNA or plasmid DNA encoding the CD3 proteins and the gamma delta T cell receptor. Cells positive for CD3 and TCR are cloned or enriched from the starting culture, cultured to increase cell numbers, and tested for activation by exposure to tumor cells and for their capacity to lyse tumor cells through cellular cytotoxicity. These methods may be used to generate NK cells or immortalized cell lines expressing the gd TCR and capable of tumor recognition and killing. Modified HSCPC or ES cells may be implanted in subjects suffering from cancer where the genetically modified NK cells are therapeutic in treating their cancer. Additionally, persons at high risk for cancer due to environmental exposure or heredity may be treated with modified NK, HSCPC or ES cells as a prophylactic therapy to reduce the chances for fatal cancer onset.

### CD3

In another aspect, methods are provided for over-expressing CD3 proteins in NK cells to facilitate gd TCR expression and consequent recognition and killing of tumor cells as a therapy for liquid and solid tumors. The method involves constructing plasmid DNA or integrating viral vectors or non-integrating viral vectors expressing the CD3 protein coding region under control of a strong promoter. Such promoters may include viral promoters similar to the CMV immediate early gene promoter, the SV40 immediate early promoter, enhanced albumin promoter, enhanced globin promoter, or any other viral or non-viral gene promoter of any origin capable of sustained, high level expression of the CD3 proteins.

The CD3 expression constructs are delivered to cells via direct RNA transfection, in the form of plasmid constructs or encoded in suitable integrating or non-integrating viral vectors. Quantitative analysis of CD3 expression is performed using antibody staining and flow cytometry, western blotting or any suitable technique known in the art, to confirm high level CD3 expression.

NK cells, cell lines, HSCPC or ES cells with high levels of CD3 protein expression are transduced with appropriate integrating or non-integrating vectors or transfected with RNA or plasmid DNA encoding the gamma delta T cell receptor. Cells positive for CD3 and gd TCR are cloned or enriched from the starting culture, cultured to increase cell numbers, and tested for activation by exposure to tumor cells and for the capacity to lyse tumor cells through cellular cytotoxicity. These methods may be used to generate NK cells or immortalized cell lines expressing the gd TCR and capable of tumor recognition and killing. Modified HSCPC or ES cells may be implanted in persons suffering from cancer with an expectation that genetically modified NK cells may be produced in vivo and will be therapeutic for their cancer. Additionally, persons at high risk for cancer due to environmental exposure or heredity may be treated with modified NK, HSCPC or ES cells as a prophylactic therapy to reduce the chances for fatal cancer onset.

### Cancer

The compositions and methods provided herein are used to treat cancer. A cell, tissue, or target may be a cancer cell, a cancerous tissue, harbor cancerous tissue, or be a subject or patient diagnosed or at risk of developing a disease or condition. In certain aspects, a cell may be an epithelial, an endothelial, a mesothelial, a glial, a stromal, or a mucosal cell. The cancer cell population can include, but is not limited to a brain, a neuronal, a blood, an endometrial, a meninges, an esophageal, a lung, a cardiovascular, a liver, a lymphoid, a breast, a bone, a connective tissue, a fat, a retinal, a thyroid, a glandular, an adrenal, a pancreatic, a stomach, an intestinal, a kidney, a bladder, a colon, a prostate, a uterine, an ovarian, a cervical, a testicular, a splenic, a skin, a smooth muscle, a cardiac muscle, or a striated muscle cell, can also include a cancer cell population from any of the foregoing, and can be associated with one or more of carcinomas, sarcomas, myelomas, leukemias, lymphomas, mixed types, a glioma, a neuroblastic tumor, or mixtures of the foregoing. In still a further aspect cancer includes, but is not limited to astrocytoma, acute myeloid leukemia, anaplastic large cell lymphoma, acute lymphoblastic leukemia, angiosarcoma, B-cell lymphoma, Burkitt's lymphoma, breast carcinoma, bladder carcinoma, carcinoma of the head and neck, cervical carcinoma, chronic lymphoblastic leukemia, chronic myeloid leukemia, colorectal carcinoma, endometrial carcinoma, esophageal squamous cell carcinoma, Ewing's sarcoma, fibrosarcoma, glioma, glioblastoma, gastrinoma, gastric carcinoma, hepatoblastoma, hepatocellular carcinoma, Kaposi's sarcoma, Hodgkin lymphoma, laryngeal squamous cell carcinoma, larynx carcinoma, leukemia, leiomyosarcoma, lipoma, liposarcoma, melanoma, mantle cell lymphoma, medulloblastoma, mesothelioma, myxofibrosarcoma, myeloid leukemia, mucosa-associated lymphoid tissue B cell lymphoma, multiple myeloma, high-risk myelodysplastic syndrome, nasopharyngeal carcinoma, neuroblastoma, neurofibroma, high-grade non-Hodgkin lymphoma, non-Hodgkin lymphoma, lung carcinoma, non-small cell lung carcinoma, ovarian carcinoma, oesophageal carcinoma, osteosarcoma, pancreatic carcinoma, pheochromocytoma, prostate carcinoma, renal cell carcinoma, retinoblastoma, rhabdomyosarcoma, salivary gland tumor, Schwanomma, small cell lung cancer, squamous cell carcinoma of the head and neck, testicular tumor, thyroid carcinoma, urothelial carcinoma, and Wilm's tumor.

The compositions and methods provided herein are also used to treat NSCLC (non-small cell lung cancer), pediatric malignancies, cervical and other tumors caused or promoted by human papilloma virus (HPV), melanoma, Barrett's esophagus (pre-malignant syndrome), adrenal and skin cancers and auto immune, neoplastic cutaneous diseases.

### Therapeutic Vectors

The construct can be delivered via known transfection and/or transduction vectors, including but not limited to lentiviral vectors, adeno-associated virus, poxvirus, herpesvirus vectors, protein and/or lipid complexes, liposomes, micelles, and the like.

Viral vectors can be preferentially targeted to cell types that are useful for the disclosed methods (i.e., tumor cells or myeloid cells). Viral vectors can be used to transduce genes into target cells owing to specific virus envelope-host cell receptor interactions and viral mechanisms for gene expression. As a result, viral vectors have been used as vehicles for the transfer of genes into many different cell types including whole embryos, fertilized eggs, isolated tissue samples, tissue targets in situ, and cultured cell lines. The ability to introduce and express foreign genes in a cell is useful for the study of gene expression, and the elucidation of cell lineages as well as providing the potential for therapeutic interventions such as gene therapy, somatic cell reprogramming of induced pluripotent stem cells, and various types of immunotherapy. Viral components from viruses like Papovaviridae (e.g. bovine papillomavirus or BPV) or Herpesviridae (e.g. Epstein Barr Virus or EBV) or Hepadnaviridae (e.g. Hepatitis B Virus or HBV) or pox vectors including vaccinia may be used in the disclosed vectors.

Lentiviral vectors are a preferred type of vector for the disclosed compositions and methods, although the disclosure is not specifically limited to lentiviral vectors. Lentivirus is a genus of viruses that can deliver a significant amount of viral nucleic acid into a host cell. Lentiviruses are characterized as having a unique ability to infect/transduce non-dividing cells, and following transduction, lentiviruses integrate their nucleic acid into the host cell's chromosomes.

Infectious lentiviruses have three main genes coding for the virulence proteins gag, pol, and env, and two regulatory genes including tat and rev. Depending on the specific serotype and virus, there may be additional accessory genes that code for proteins involved in regulation, synthesis, and/or processing viral nucleic acids and other replicative functions.

Moreover, lentiviruses contain long terminal repeat (LTR) regions, which may be approximately 600 nt long. LTRs may be segmented into U3, R, and U5 regions. LTRs can mediate integration of retroviral DNA into the host chromosome via the action of integrase. Alternatively, without functioning integrase, the LTRs may be used to circularize the viral nucleic acid.

Viral proteins involved in early stages of lentivirus replication include reverse transcriptase and integrase. Reverse transcriptase is the virally encoded, RNA-dependent DNA polymerase. The enzyme uses a viral RNA genome as a template for the synthesis of a complementary DNA copy. Reverse transcriptase also has RNaseH activity for destruction of the RNA-template. Integrase binds both the viral cDNA generated by reverse transcriptase and the host DNA. Integrase processes the LTR before inserting the viral genome into the host DNA. Tat acts as a trans-activator during transcription to enhance initiation and elongation. The rev responsive element acts post-transcriptionally, regulating mRNA splicing and transport to the cytoplasm.

Viral vectors, in general, comprise glycoproteins and the various glycoproteins may provide specific affinities. For instance, VSVG peptides can increase transfection into myeloid cells. Alternatively, viral vectors can also have targeting moieties, such as antibodies, attached to their shell peptides. Targeting antibodies can be specific for antigens that are overexpressed on a tumor, for instance, like HER-2, PSA, CEA, M2-PK, and CA19-9.

Other viral vector specificities are also known in the art and can be used to target a particular population of cells. For example, poxvirus vectors target to macrophages and dendritic cells.

### Lentiviral Vector System

A lentiviral virion (particle) is expressed by a vector system encoding the necessary viral proteins to produce a virion (viral particle). There is at least one vector containing a nucleic acid sequence encoding the lentiviral pol proteins necessary for reverse transcription and integration, operably linked to a promoter. In another embodiment, the pol proteins are expressed by multiple vectors. There is also a vector containing a nucleic acid sequence encoding the lentiviral gag proteins necessary for forming a viral capsid operably linked to a promoter. In an embodiment, this gag nucleic acid sequence is on a separate vector than at least some of the poi nucleic acid sequence. In another embodiment, the gag nucleic acid is on a separate vector from all the poi nucleic acid sequences that encode pol proteins.

Numerous modifications can be made to the vectors, which are used to create the particles to further minimize the chance of obtaining wild type revertants. These include, but are not limited to, deletions of the U3 region of the LTR, tat deletions and matrix (MA) deletions.

The gag, poi and env vector(s) do not contain nucleotides from the lentiviral genome that package lentiviral RNA, referred to as the lentiviral packaging sequence.

The vector(s) forming the particle preferably do not contain a nucleic acid sequence from the lentiviral genome that expresses an envelope protein. Preferably, a separate vector that contains a nucleic acid sequence encoding an envelope protein operably linked to a promoter is used. This env vector also does not contain a lentiviral packaging sequence. In one embodiment the env nucleic acid sequence encodes a lentiviral envelope protein.

In another embodiment the envelope protein is not from the lentivirus, but from a different virus. The resultant particle is referred to as a pseudotyped particle. By appropriate selection of envelopes one can “infect” virtually any cell. For example, one can use an env gene that encodes an envelope protein that targets an endocytic compartment such as that of the influenza virus, VSV-G, alpha viruses (Semliki forest virus, Sindbis virus), arenaviruses (lymphocytic choriomeningitis virus), flaviviruses (tick-borne encephalitis virus, Dengue virus, hepatitis C virus, GB virus), rhabdoviruses (vesicular stomatitis virus, rabies virus), paramyxoviruses (mumps or measles) and orthomyxoviruses (influenza virus). Other envelopes that can preferably be used include those from Moloney Leukemia Virus such as MLV-E, MLV-A and GALV. These latter envelopes are particularly preferred where the host cell is a primary cell. Other envelope proteins can be selected depending upon the desired host cell. For example, targeting specific receptors such as a dopamine receptor can be used for brain delivery. Another target can be vascular endothelium. These cells can be targeted using a filovirus envelope. For example, the GP of Ebola, which by post-transcriptional modification become the GP, and GP 2 glycoproteins. In another embodiment, one can use different lentiviral capsids with a pseudotyped envelope (for example, FIV or SHIV [U.S. Pat. No. 5,654,195]). A SHIV pseudotyped vector can readily be used in animal models such as monkeys.

As detailed herein, a lentiviral vector system typically includes at least one helper plasmid comprising at least one of a gag, pol, or rev gene. Each of the gag, pol and rev genes may be provided on individual plasmids, or one or more genes may be provided together on the same plasmid. In one embodiment, the gag, pol, and rev genes are provided on the same plasmid (e.g., FIG. 2). In another embodiment, the gag and pol genes are provided on a first plasmid and the rev gene is provided on a second plasmid (e.g., FIG. 3). Accordingly, both 3-vector and 4-vector systems can be used to produce a lentivirus as described in the Examples section and elsewhere herein. The therapeutic vector, the envelope plasmid and at least one helper plasmid are transfected into a packaging cell line. A non-limiting example of a packaging cell line is the 293T/17 HEK cell line. When the therapeutic vector, the envelope plasmid, and at least one helper plasmid are transfected into the packaging cell line, a lentiviral particle is ultimately produced.

In another aspect, a lentiviral vector system for expressing a lentiviral particle is disclosed. The system includes a lentiviral vector as described herein; an envelope plasmid for expressing an envelope protein optimized for infecting a cell; and at least one helper plasmid for expressing gag, pol, and rev genes, wherein when the lentiviral vector, the envelope plasmid, and the at least one helper plasmid are transfected into a packaging cell line, a lentiviral particle is produced by the packaging cell line, wherein the lentiviral particle is capable of inhibiting production of chemokine receptor CCR5 or targeting an HIV RNA sequence.

In another aspect, and as detailed in FIGS. 2 and 3, the lentiviral vector expressing Vγ9Vδ2 T cell receptor, can include the following elements: hybrid 5′ long terminal repeat (RSV/5′ LTR) (SEQ ID NOS: 44-45), Psi sequence (RNA packaging site) (SEQ ID NO: 46), RRE (Rev-response element) (SEQ ID NO: 47), cPPT (polypurine tract) (SEQ ID NO: 48), EF1 alpha promoter (SEQ ID NO: 7), VG9 (SEQ ID NOs: 1 and 3), IRES (SEQ ID NO: 6) Vd2 (SEQ ID NOs: 2 and 4) Woodchuck Post-Transcriptional Regulatory Element (WPRE) (SEQ ID NO: 54), and 3′ Delta LTR (SEQ ID NO: 55). In another aspect, sequence variation, by way of substitution, deletion, addition, or mutation can be used to modify the sequences references herein.

In another aspect, and as detailed in FIG. 4, the lentiviral vector expressing CD3, can include the following elements: hybrid 5′ long terminal repeat (RSV/5′ LTR) (SEQ ID NOS: 44-45), Psi sequence (RNA packaging site) (SEQ ID NO: 46), RRE (Rev-response element) (SEQ ID NO: 47), cPPT (polypurine tract) (SEQ ID NO: 48), CMV promoter (SEQ ID NO: 8), CD3 (SEQ ID NO: 9), Woodchuck Post-Transcriptional Regulatory Element (WPRE) (SEQ ID NO: 54), and 3′ Delta LTR (SEQ ID NO: 55). In another aspect, sequence variation, by way of substitution, deletion, addition, or mutation can be used to modify the sequences references herein.

In another aspect, and as detailed in FIG. 4, the lentiviral vector expressing FDPS shRNA, can include the following elements: hybrid 5′ long terminal repeat (RSV/5′ LTR) (SEQ ID NOS: 44-45), Psi sequence (RNA packaging site) (SEQ ID NO: 46), RRE (Rev-response element) (SEQ ID NO: 47), cPPT (polypurine tract) (SEQ ID NO: 48), H1 promoter (SEQ ID NO: 49), FDPS shRNA (SEQ ID NOS: 50-53), Woodchuck Post-Transcriptional Regulatory Element (WPRE) (SEQ ID NO: 54), and 3′ Delta LTR (SEQ ID NO: 55). In another aspect, sequence variation, by way of substitution, deletion, addition, or mutation can be used to modify the sequences references herein.

In another aspect, and as detailed herein, a helper plasmid has been designed to include the following elements: CAG promoter (SEQ ID NO: 56); HIV gag (SEQ ID NO: 57); HIV pol (SEQ ID NO: 58); HIV Integrase (SEQ ID NO: 59); HIV RRE (SEQ ID NO: 60); and HIV Rev (SEQ ID NO: 61). In another aspect, the helper plasmid may be modified to include a first helper plasmid for expressing the gag and pol genes, and a second and separate plasmid for expressing the rev gene. In another aspect, sequence variation, by way of substitution, deletion, addition, or mutation can be used to modify the sequences references herein.

In another aspect, and as detailed herein, an envelope plasmid has been designed to include the following elements being from left to right: CMV promoter (SEQ ID NO: 62) and vesicular stomatitis virus G glycoprotein (VSV-G) (SEQ ID NO: 63). In another aspect, sequence variation, by way of substitution, deletion, addition, or mutation can be used to modify the sequences references herein.

In another aspect, the plasmids used for lentiviral packaging can be modified with similar elements and the intron sequences could potentially be removed without loss of vector function. For example, the following elements can replace similar elements in the plasmids that comprise the packaging system: Elongation Factor-1 alpha (EF1α), phosphoglycerate kinase (PGK), and ubiquitin C (UbC) promoters can replace the CMV or CAG promoter. SV40 poly A and bGH poly A can replace the rabbit beta globin poly A. The HIV sequences in the helper plasmid can be constructed from different HIV strains or clades. The VSV-G glycoprotein can be substituted with membrane glycoproteins from feline endogenous virus (RD114), gibbon ape leukemia virus (GALV), Rabies (FUG), lymphocytic choriomeningitis virus (LCMV), influenza A fowl plague virus (FPV), Ross River alphavirus (RRV), murine leukemia virus 10A1 (MLV), or Ebola virus (EboV).

Of note, lentiviral packaging systems can be acquired commercially (e.g., Lenti-vpak packaging kit from OriGene Technologies, Inc., Rockville, MD), and can also be designed as described herein. Moreover, it is within the ordinary skill of a person skilled in the art to substitute or modify aspects of a lentiviral packaging system to improve any number of relevant factors, including the production efficiency of a lentiviral particle.

### Doses and Dosage Forms

The disclosed vectors allow for short, medium, or long-term expression of genes or sequences of interest and episomal maintenance of the disclosed vectors. Accordingly, dosing regimens may vary based upon the condition being treated and the method of administration.

In one embodiment, transduction vectors may be administered to a subject in need in varying doses. Specifically, a subject may be administered about ≥10⁶ infectious doses (where 1 dose is needed on average to transduce 1 target cell). More specifically, a subject may be administered about ≥10⁷, about ≥10⁸, about ≥10⁹, or about ≥10¹⁰ infectious doses, or any number of doses in-between these values. Upper limits of transduction vector dosing will be determined for each disease indication and will depend on toxicity/safety profiles for each individual product or product lot.

Additionally, a vector of the present disclosure may be administered periodically, such as once or twice a day, or any other suitable time period. For example, vectors may be administered to a subject in need once a week, once every other week, once every three weeks, once a month, every other month, every three months, every six months, every nine months, once a year, every eighteen months, every two years, every thirty months, or every three years.

In one embodiment, the disclosed vectors are administered as a pharmaceutical composition. In some embodiments, the pharmaceutical composition comprising the disclosed vectors can be formulated in a wide variety of dosage forms, including but not limited to nasal, pulmonary, oral, topical, or parenteral dosage forms for clinical application. Each of the dosage forms can comprise various solubilizing agents, disintegrating agents, surfactants, fillers, thickeners, binders, diluents such as wetting agents or other pharmaceutically acceptable excipients. The pharmaceutical composition comprising a vector can also be formulated for injection, insufflation, infusion, or intradermal exposure. For instance, an injectable formulation may comprise the disclosed vectors in an aqueous or non-aqueous solution at a suitable pH and tonicity.

The disclosed vectors may be administered to a subject via direct injection into a tumor site or at a site of infection. In some embodiments, the vectors can be administered systemically. In some embodiments, the vectors can be administered via guided cannulation to tissues immediately surrounding the sites of tumor or infection.

The disclosed vector compositions can be administered using any pharmaceutically acceptable method, such as intranasal, buccal, sublingual, oral, rectal, ocular, parenteral (intravenously, intradermally, intramuscularly, subcutaneously, intraperitoneally), pulmonary, intravaginal, locally administered, topically administered, topically administered after scarification, mucosally administered, via an aerosol, in semi-solid media such as agarose or gelatin, or via a buccal or nasal spray formulation.

Further, the disclosed vector compositions can be formulated into any pharmaceutically acceptable dosage form, such as a solid dosage form, tablet, pill, lozenge, capsule, liquid dispersion, gel, aerosol, pulmonary aerosol, nasal aerosol, ointment, cream, semi-solid dosage form, a solution, an emulsion, and a suspension. Further, the composition may be a controlled release formulation, sustained release formulation, immediate release formulation, or any combination thereof. Further, the composition may be a transdermal delivery system.

In some embodiments, the pharmaceutical composition comprising a vector can be formulated in a solid dosage form for oral administration, and the solid dosage form can be powders, granules, capsules, tablets or pills. In some embodiments, the solid dosage form can include one or more excipients such as calcium carbonate, starch, sucrose, lactose, microcrystalline cellulose or gelatin. In addition, the solid dosage form can include, in addition to the excipients, a lubricant such as talc or magnesium stearate. In some embodiments, the oral dosage form can be immediate release, or a modified release form. Modified release dosage forms include controlled or extended release, enteric release, and the like. The excipients used in the modified release dosage forms are commonly known to a person of ordinary skill in the art.

In a further embodiment, the pharmaceutical composition comprising a vector can be formulated as a sublingual or buccal dosage form. Such dosage forms comprise sublingual tablets or solution compositions that are administered under the tongue and buccal tablets that are placed between the cheek and gum.

In some embodiments, the pharmaceutical composition comprising a vector can be formulated as a nasal dosage form. Such dosage forms of the present disclosure comprise solution, suspension, and gel compositions for nasal delivery.

In some embodiments, the pharmaceutical composition comprising a vector can be formulated in a liquid dosage form for oral administration, such as suspensions, emulsions or syrups.

In some embodiments, the liquid dosage form can include, in addition to commonly used simple diluents such as water and liquid paraffin, various excipients such as humectants, sweeteners, aromatics or preservatives. In particular embodiments, the composition comprising vectors can be formulated to be suitable for administration to a pediatric patient.

In some embodiment, the pharmaceutical composition can be formulated in a dosage form for parenteral administration, such as sterile aqueous solutions, suspensions, emulsions, non-aqueous solutions or suppositories. In some embodiments, the solutions or suspensions can include propyleneglycol, polyethyleneglycol, vegetable oils such as olive oil or injectable esters such as ethyl oleate.

The dosage of the pharmaceutical composition can vary depending on the patient's weight, age, gender, administration time and mode, excretion rate, and the severity of disease.

In some embodiments, the treatment of cancer is accomplished by guided direct injection of the disclosed vector constructs into tumors, using needle, or intravascular cannulation. In some embodiments, the disclosed vectors are administered into the cerebrospinal fluid, blood or lymphatic circulation by venous or arterial cannulation or injection, intradermal delivery, intramuscular delivery or injection into a draining organ near the site of disease.

The following examples are given to illustrate exemplary aspects and embodiments of the disclosure. It should be understood, however, that the aspects and embodiments not to be limited to the specific conditions or details described in these examples. All printed publications referenced herein are specifically incorporated by reference.

### EXAMPLES

### Example 1: Development of a Lentiviral Vector System

Lentiviral particles were produced in 293T/17 HEK cells (purchased from American Type Culture Collection, Manassas, VA) following transfection with the therapeutic vector, the envelope plasmid, and the helper plasmid. The transfection of 293T/17 HEK cells, which produced functional viral particles, employed the reagent Poly(ethylenimine) (PEI) to increase the efficiency of plasmid DNA uptake. The plasmids and DNA were initially added separately in culture medium without serum in a ratio of 3:1 (mass ratio of PEI to DNA). After 2-3 days, cell medium was collected and lentiviral particles were purified by high-speed centrifugation and/or filtration followed by anion-exchange chromatography. The concentration of lentiviral particles can be expressed in terms of transducing units/ml (TU/ml). The determination of TU was accomplished by measuring HIV p24 levels in culture fluids (p24 protein is incorporated into lentiviral particles), measuring the number of viral DNA copies per cell by quantitative PCR, or by infecting cells and using light (if the vectors encode luciferase or fluorescent protein markers).

As mentioned above, a 3-vector system (i.e., a 2-vector lentiviral packaging system) was designed for the production of lentiviral particles. A schematic of the 3-vector system is shown in FIG. 2. Briefly, and with reference to FIG. 2, the top-most vector is a helper plasmid, which, in this case, includes Rev. The vector appearing in the middle of FIG. 2 is the envelope plasmid. The bottom-most vector is the therapeutic vector, as described herein.

Referring more specifically to FIG. 2, the Helper plus Rev plasmid includes a CAG enhancer (SEQ ID NO: 10); a CAG promoter (SEQ ID NO: 11); a chicken beta actin intron (SEQ ID NO: 12); a HIV gag (SEQ ID NO: 13); a HIV Pol (SEQ ID NO: 14); a HIV Integrase (SEQ ID NO: 15); a HIV RRE (SEQ ID NO: 16); a HIV Rev (SEQ ID NO: 17); and a rabbit beta globin poly A (SEQ ID NO: 18).

The Envelope plasmid includes a CMV promoter (SEQ ID NO: 19); a beta globin intron (SEQ ID NO: 20); a VSV-G (SEQ ID NO: 12); and a rabbit beta globin poly A (SEQ ID NO: 21).

Synthesis of a 2-Vector Lentiviral Packaging System Including Helper (Plus Rev) and Envelope Plasmids.

Materials and Methods:

Construction of the helper plasmid: The helper plasmid was constructed by initial PCR amplification of a DNA fragment from the pNL4-3 HIV plasmid (NIH Aids Reagent Program) containing Gag, Pol, and Integrase genes. Primers were designed to amplify the fragment with EcoRI and NotI restriction sites which could be used to insert at the same sites in the pCDNA3 plasmid (Invitrogen). The forward primer was SEQ ID NO: 22 and reverse primer was SEQ ID NO: 23. The sequence for the Gag, Pol, Integrase fragment was SEQ ID NO: 24.

Next, a DNA fragment containing the Rev, RRE, and rabbit beta globin poly A sequence with XbaI and XmaI flanking restriction sites was synthesized by MWG Operon. The DNA fragment was then inserted into the plasmid at the XbaI and XmaI restriction sites. The DNA sequence was SEQ ID NO: 25.

Finally, the CMV promoter of pCDNA3.1 was replaced with the CAG enhancer/promoter plus a chicken beta actin intron sequence. A DNA fragment containing the CAG enhancer/promoter/intron sequence with MluI and EcoRI flanking restriction sites was synthesized by MWG Operon. The DNA fragment was then inserted into the plasmid at the MluI and EcoRI restriction sites. The DNA sequence was SEQ ID NO: 26.

Construction of the VSV-G Envelope Plasmid:

The vesicular stomatitis Indiana virus glycoprotein (VSV-G) sequence was synthesized by MWG Operon with flanking EcoRI restriction sites. The DNA fragment was then inserted into the pCDNA3.1 plasmid (Invitrogen) at the EcoRI restriction site and the correct orientation was determined by sequencing using a CMV specific primer. The DNA sequence was SEQ ID NO: 27.

A 4-vector system (i.e., a 3-vector lentiviral packaging system) has also been designed and produced using the methods and materials described herein. A schematic of the 4-vector system is shown in FIG. 3. Briefly, and with reference to FIG. 3, the top-most vector is a helper plasmid, which, in this case, does not include Rev. The vector second from the top is a separate Rev plasmid. The vector second from the bottom is the envelope plasmid. The bottom-most vector is the previously described therapeutic vector.

Referring, in part, to FIG. 3, the Helper plasmid includes a CAG enhancer (SEQ ID NO: 10); a CAG promoter (SEQ ID NO: 11); a chicken beta actin intron (SEQ ID NO: 12); a HIV gag (SEQ ID NO: 13); a HIV Pol (SEQ ID NO: 14); a HIV Integrase (SEQ ID NO: 15); a HIV RRE (SEQ ID NO: 16); and a rabbit beta globin poly A (SEQ ID NO: 18).

The Rev plasmid includes an RSV promoter (SEQ ID NO: 28); a HIV Rev (SEQ ID NO: 29); and a rabbit beta globin poly A (SEQ ID NO: 18).

The Envelope plasmid includes a CMV promoter (SEQ ID NO: 19); a beta globin intron (SEQ ID NO: 20); a VSV-G (SEQ ID NO: 12); and a rabbit beta globin poly A (SEQ ID NO: 18).

Synthesis of a 3-Vector Lentiviral Packaging System Including Helper, Rev, and Envelope Plasmids.

Materials and Methods:

Construction of the Helper Plasmid without Rev:

The Helper plasmid without Rev was constructed by inserting a DNA fragment containing the RRE and rabbit beta globin poly A sequence. This sequence was synthesized by MWG Operon with flanking NotI and StuI restriction sites. The RRE/rabbit poly A beta globin sequence was then inserted into the Helper plasmid at the NotI and StuI restriction sites. The DNA sequence SEQ ID NO: 67.

Construction of the Rev Plasmid:

The RSV promoter and HIV Rev sequence was synthesized as a single DNA fragment by MWG Operon with flanking MfeI and XbaI restriction sites. The DNA fragment was then inserted into the pCDNA3.1 plasmid (Invitrogen) at the MfeI and XbaI restriction sites in which the CMV promoter is replaced with the RSV promoter. The DNA sequence was SEQ ID NO: 30.

The plasmids for the 2-vector and 3-vector packaging systems could be modified with similar elements and the intron sequences could potentially be removed without loss of vector function. For example, the following elements could replace similar elements in the 2-vector and 3-vector packaging system:

Promoters: Elongation Factor-1 alpha (EF1α) (SEQ ID NO: 31), phosphoglycerate kinase (PGK) (SEQ ID NO: 32), and ubiquitin C (UbC) (SEQ ID NO: 33) can replace the CMV (SEQ ID NO: 19) or CAG promoter (SEQ ID NO: 11). These sequences can also be further varied by addition, substitution, deletion or mutation.

Poly A sequences: SV40 poly A (SEQ ID NO: 34) and bGH poly A (SEQ ID NO: 35) can replace the rabbit beta globin poly A (SEQ ID NO: 18). These sequences can also be further varied by addition, substitution, deletion or mutation.

HIV Gag, Pol, and Integrase sequences: The HIV sequences in the Helper plasmid can be constructed from different HIV strains or clades. For example, HIV Gag (SEQ ID NO: 13); HIV Pol (SEQ ID NO: 14); and HIV Integrase (SEQ ID NO: 15) from the Bal strain can be interchanged with the gag, pol, and int sequences contained in the helper/helper plus Rev plasmids as outlined herein. These sequences can also be further varied by addition, substitution, deletion or mutation.

Envelope: The VSV-G glycoprotein can be substituted with membrane glycoproteins from feline endogenous virus (RD114) (SEQ ID NO: 36), gibbon ape leukemia virus (GALV) (SEQ ID NO: 37), Rabies (FUG) (SEQ ID NO: 38), lymphocytic choriomeningitis virus (LCMV) (SEQ ID NO: 39), influenza A fowl plague virus (FPV) (SEQ ID NO: 40), Ross River alphavirus (RRV) (SEQ ID NO: 41), murine leukemia virus 10A1 (MLV) (SEQ ID NO: 42), or Ebola virus (EboV) (SEQ ID NO: 43). Sequences for these envelopes are identified in the sequence portion herein. Further, these sequences can also be further varied by addition, substitution, deletion or mutation.

In summary, the 3-vector versus 4-vector systems can be compared and contrasted, in part, as follows. The 3-vector lentiviral vector system contains: 1. Helper plasmid: HIV Gag, Pol, Integrase, and Rev/Tat; 2. Envelope plasmid: VSV-G/FUG envelope; and 3. Therapeutic vector: RSV 5′LTR, Psi Packaging Signal, Gag fragment, RRE, Env fragment, cPPT, WPRE, and 3′δ LTR. The 4-vector lentiviral vector system contains: 1. Helper plasmid: HIV Gag, Pol, and Integrase; 2. Rev plasmid: Rev; 3. Envelope plasmid: VSV-G/FUG envelope; and 4. Therapeutic vector: RSV 5′LTR, Psi Packaging Signal, Gag fragment, RRE, Env fragment, cPPT, WPRE, and 3′ delta LTR. Sequences corresponding with the above elements are identified in the sequence listings portion herein.

### Example 2: Transducing NK92 Cells with γδ T Cell Receptors and CD3 Proteins

A DNA fragment of the Homo sapiens T cell receptor gamma 9 variable chain (TRGV9) (Gen Bank: KC170727.1) was synthesized by Integrated DNA Technologies (IDT) with flanking BsrGI and NotI restriction enzyme sites. A DNA fragment of the Homo sapiens T cell receptor delta chain (TRDV2) (Gen Bank: AY312956.1) with an upstream internal ribosomal entry site (IRES) sequence was synthesized with flanking NotI and NsiI restriction enzyme sites. TRGV9 was digested with BsrGI/NotI enzymes and TRDV2 was digested with NotI/NsiI enzymes and ligated to a lentivirus plasmid under control of the EF1α promoter. The TRGV9 fragment was inserted into the lentivirus plasmid before inserting the TRDV2 fragment. Similarly, a DNA fragment of Homo sapiens CD3 was synthesized with XbaI and SalI restriction enzyme sites and inserted into a lentivirus plasmid under control of the CMV promoter.

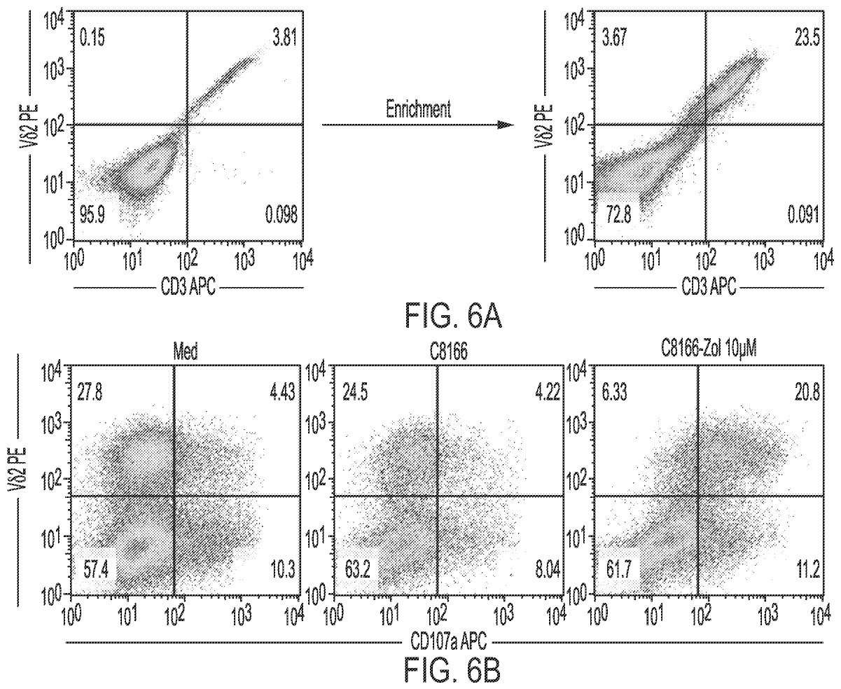

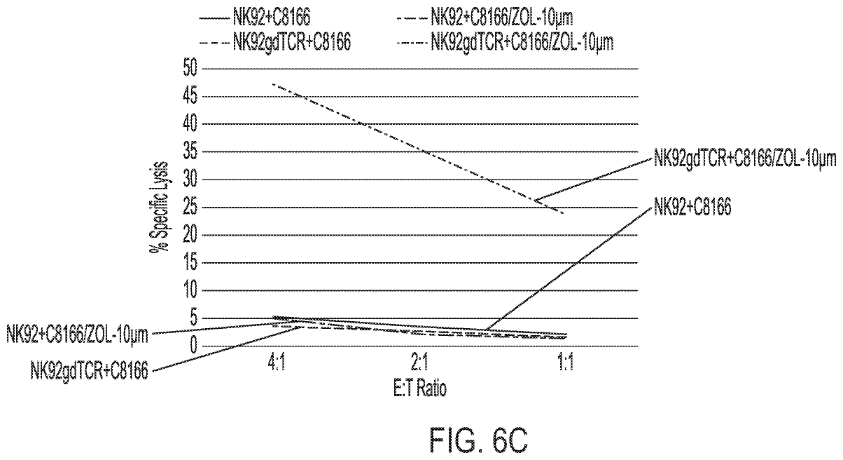

The lentivirus plasmid was digested with either BsrGI/NotI or NotI/NsiI restriction enzymes. The digested product was electrophoresed on a 1% agarose gel (Thermo Fisher Scientific), excised, and extracted from the gel with the PureLink DNA gel extraction kit (Thermo Fisher Scientific). The DNA concentration was determined and then mixed with the digested DNA fragment using a vector to insert ratio of 3:1. The mixture was ligated with T4 DNA ligase for 16 hours at room temperature. 3 μL of the ligation mix was added to 23 μL of STBL3 competent bacterial cells. Transformation was carried out by heat-shock at 42 degrees Celsius. Bacterial cells were streaked onto agar plates containing ampicillin and then colonies were expanded in LB broth. To check for insertion of the DNA fragments, plasmid DNA was extracted from harvested bacteria cultures with the DNA plasmid mini prep kit (Thermo Fisher Scientific). The inserted DNA fragments were verified by DNA sequencing (Eurofins Genomics). The lentivirus vectors containing a verified sequence were then used to package lentiviral particles in 293T cells to test for their ability to express either the Vγ2Vδ2 TCR or CD3 by antibody detection with a flow cytometer. For transduction of NK92 cells, the cells were seeded at 2×10⁵ cells per mL in a 24 well plate and transduced with 5 moi of the lentivirus vector expressing CD3. After 4 days, CD3 expression was detected with an anti-CD3 antibody by flow cytometry. The NK92 cells expressing CD3 were then transduced with a lentivirus vector expressing a Vγ2Vδ2 TCR. After 4 days, expression of the Vγ2Vδ2 TCR was detected with an anti-Vγ9 or anti-Vδ2 antibody by flow cytometry (see left panel of FIG. 6A).

### Example 3: Enrichment of NK92 Cells Containing Vγ9Vδ2 T Cell Receptors and CD3 Protein Using Anti-V62 Bead Selection

NK92 cells that expressed Vγ9Vδ2 T cell receptors and CD3 protein were enriched using anti-Vδ2 beads.

Experimental Methods:

NK92 cells were transduced with a lentivirus vector expressing Vγ9Vδ2 T cell receptor (TCR) (SEQ ID NO: 5) and a lentivirus vector expressing CD3 proteins (SEQ ID NO: 9) at a moi of 5. After 5 days, the expression of CD3 and Vγ9Vδ2 TCR were measured by flow cytometry. Cells were washed, resuspended in 50-100 μl of staining buffer, and stained with mouse anti-human Vδ2-phycoerythrin (PE) clone B6 and mouse anti-human CD3-allophycocyanin (APC) clone UCHT1. Cells were washed with staining buffer and resuspended. Data for at least 1×10⁴ lymphocytes (gated on the basis of forward- and side-scatter profiles) were acquired from each sample on a FACSCalibur flow cytometer (BD Biosciences). All samples were analyzed using FlowJo software.

As shown in FIG. 6A, without enrichment, the frequency of CD3 and Vγ9Vδ2 TCR positive cells was 3.81% (see left panel of FIG. 6A). To characterize the transduced cells, cells were further enriched with an anti-Vγ9Vδ2 TCR antibody and magnetic beads selection. After enrichment, the frequency of CD3 and Vγ9Vδ2 TCR positive cells was 23.5% (see right panel of FIG. 6A).

### Example 4: NK92 Cells Engineered with Vγ9Vδ2 T Cell Receptors are Responsive to C8166 Cells Treated with Zoledronic Acid

NK92 cells engineered with Vγ9Vδ2 T cell receptors were tested to determine whether they were responsive to C8166 cells. C8166 cells are a model for human T cell leukemia. CD107a was used as a marker for cell activity.

Experimental Methods:

Zolendronic acid inhibits farnesyl diphosphate synthase (FDPS) and this leads to isopentenyl pyrophosphate (IPP) accumulation in the cells that increases recognition by Vγ9Vδ2 TCR. If CD3 and Vγ9Vδ2 TCR are functional in NK92 cells, the Vγ9Vδ2 TCR positive NK92 will respond to zoledronic acid treated cells and express CD107a on the cell surface as a marker of degranulation. C8166 cells were treated without or with zoledronic acid (10 μM) for 24 hours. The enriched NK92 cells were treated with medium, C8166 tumor cells, or 10 μM zoledronic acid treated C8166 cells at a 1:1 ratio for 4 hours. CD107a was measured by flow cytometry. Data for at least 1×10⁴ lymphocytes (gated on the basis of forward- and side-scatter profiles) were acquired for each sample on a FACSCalibur flow cytometer (BD Biosciences). All samples were analyzed using FlowJo software.

As shown in FIG. 6B, Vγ9Vδ2 TCR positive NK92 cells responded to zoledronic acid treated C8166 cells through increased expression of CD107a, with 20.8% of the cells positive for CD107a. However, non-zoledronic treated C8166 cells failed to induce a CD107a response, as only 4.22% of Vγ9Vδ2 TCR positive NK92 were positive for expression of CD107a, which can be compared to 4.43% of Vγ9Vδ2 TCR positive NK92 that were positive for expression of CD107a in control treatments (see FIG. 6B).

### Example 5: Exposing Zoledronic Acid Treated C8166 Cells to NK92 Cells Engineered with Vγ9Vδ2 T Cell Receptors Resulted in Cell Cytolysis

NK92 cells engineered with Vγ9Vδ2 receptor were tested for their ability to lyse C8166 cells.

Experimental Methods:

A fluorometric cytotoxicity assay with calcein-acetoxymethyl (Molecular Probes, Eugene, OK) was used to measure cytotoxicity against target cells. C8166 cells were treated without or with Zolendronic Acid (10 μM) for 24 hours and used as target cells. Target cells were labelled for 15 min with 2 mM calcein-acetoxymethyl at 37° C., then washed once with PBS. Cells were combined at various effector-to-target (E:T) ratios in 96-well round-bottom microtitre plates (Corning, NY) and incubated at 37° C. in 5% CO₂ for 4 hr; assays were performed in triplicate. After incubation, supernatants were transferred to a 96-well flat-bottom microtitre plate and calcein content was measured using a BioTek Synergy HT plate reader (485/535 nm). Percent specific lysis was calculated as: (test release−spontaneous release)/(maximum release−spontaneous release)×100.

The cytotoxicity of NK92 cells or Vγ9Vδ2 TCR engineered NK92 cells against C8166 cells or Zoledronic acid-treated C8166 cells was evaluated at several effector to target (E:T) ratios. Triplicate wells were used for each condition. As shown in FIG. 6C, zoledronic acid-treated C8166 cells exposed to NK92 cells that expressed the Vγ9Vδ2 TCR, resulted in the highest level of cell lysis (see line that corresponds to NK92gdTCR+C8166/ZOL-10 μM). Specifically, at a 4:1 effector to target ratio, between 45% and 50% of the C8166 cells were lysed; cell lysis was reduced to around 25% when the effector to target ratio was reduced to 1:1 (see FIG. 6C).

### Example 6: Isolating Individual NK92 Cells that were Positive for Transduction Through Limiting Dilution Single Cell Cloning

Not all NK92 cells were transduced with lentivirus vector expressing the Vγ2Vδ2 TCR. As a result, individual cells positive for transduction were isolated, and sublines were grown from those isolated cells. Isolation of positively transduced cells and growing of sublines from those isolated cells was carried out using limiting dilution single cell cloning.

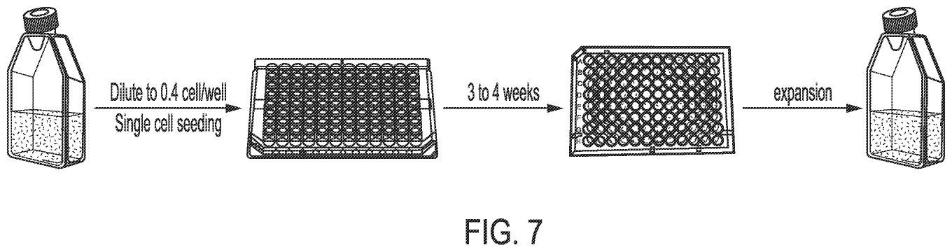

NK92 cells were transduced with lentivirus vectors expressing γδ T Cell receptor genes or CD3 protein genes. The resulting cells were diluted and plated in 96 well plates at a density of 0.4 cells per well. This condition ensures that only single cells are placed into individual wells and will grow as cloned cell lines. After 3 to 4 weeks of culture, single cells proliferated and their growth caused culture medium to become yellow in color. The cells from individual wells were transferred to 25 mL culture flasks to allow continued growth and expansion of the cloned cell line. Cells from the 25 mL cultures were used to characterize the phenotype, based on cell surface protein expression, and biological function, based on cellular cytotoxicity assays. A schematic of this method is shown in FIG. 7.

### Example 7: Screening of Sublines of NK92 Vγ9Vδ2 T Cell Receptor Clones, Generated by Limited Dilution Cloning, Showed that the NK92-γδTCR-5 Subline Produced a High Expression of CD107a and TNFα in the Presence of Tumor Cells

Sublines of NK92 cells that expressed Vγ9Vδ2 T cell receptor were screened through exposing the NK92 cells to C8166 cells or zoledronic acid treated C8166 cells. CD107a and TNFα were used as markers to detect cell activity.

Experimental Methods:

C8166 cells were treated without or with zoledronic acid (10 μM) for 24 hours. The selected NK92 cell clones that express the Vγ9Vδ2 T cell Receptor were treated with C8166 cells, or 10 μM zoledronic acid treated C8166 cells at a 1:1 ratio for 4 hours. CD107a or TNFα was measured by flow cytometry. For detecting CD107a, cells were washed, resuspended in 50-100 μl of staining buffer, and stained with mouse anti-human Vδ2-PE clone B6 and mouse anti-human CD107a-APC clone H4A3. For detecting intracellular TNFα, cells were stained with mouse anti-human Vδ2-PE clone B6 then fixed, permeabilized, and incubated for 45 min at 4° C. with mouse anti-human TNFα-APC. Intracellular staining solutions were obtained from the Cytofix/Cytoperm Kit (BD Biosciences). Cells were washed with staining buffer and resuspended. Data for at least 1×10⁴ lymphocytes (gated on the basis of forward- and side-scatter profiles) were acquired from each sample on a FACSCalibur flow cytometer (BD Biosciences). All data were analyzed using FlowJo software.

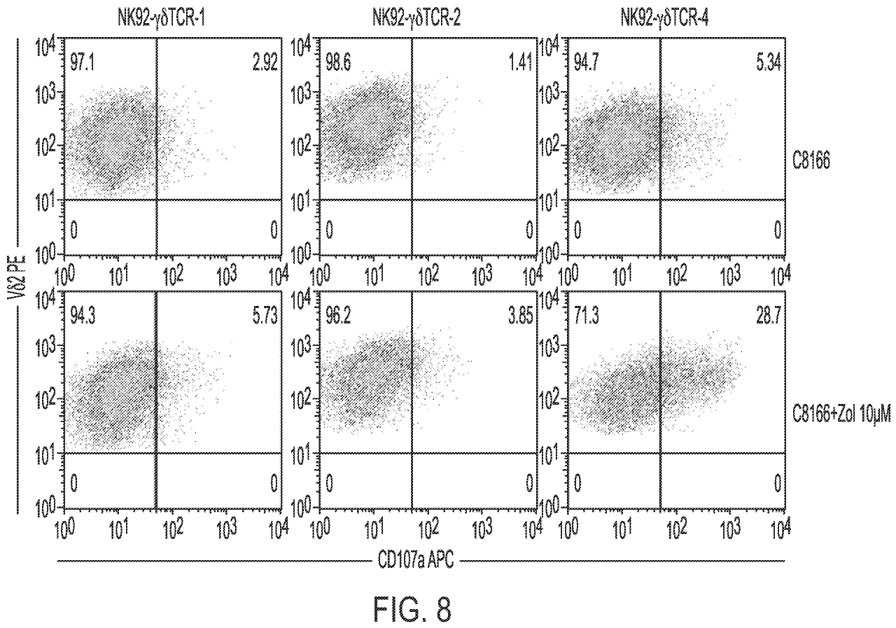

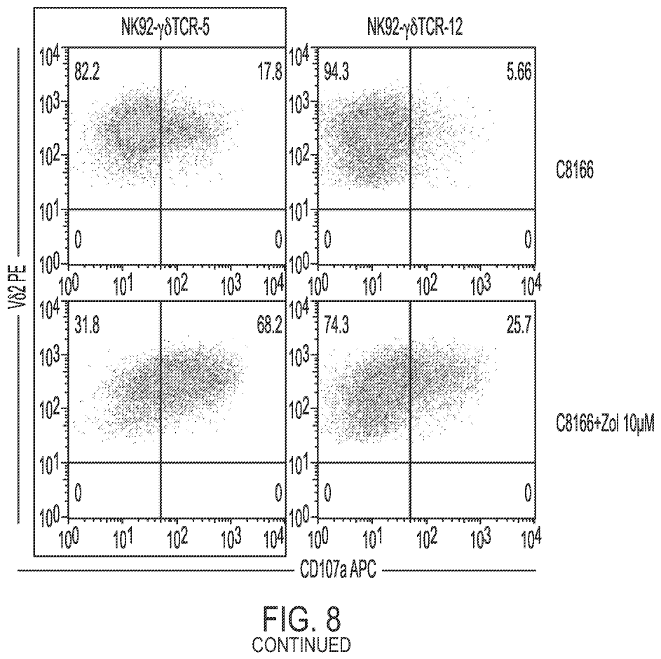

As shown in FIG. 8, when CD107a was used as the marker, the NK92-γδTCR-5 subline showed the strongest response. Specifically, when the NK92-γδTCR-5 subline was exposed to C8166 cells, 17.8% of the cells were positive for the CD107a marker (see FIG. 8). This percentage of CD107a positive cells increased to 68.2%, when the NK92-γδTCR subline was exposed to CD1866 cells that had been treated with zoledronic acid (see FIG. 8).

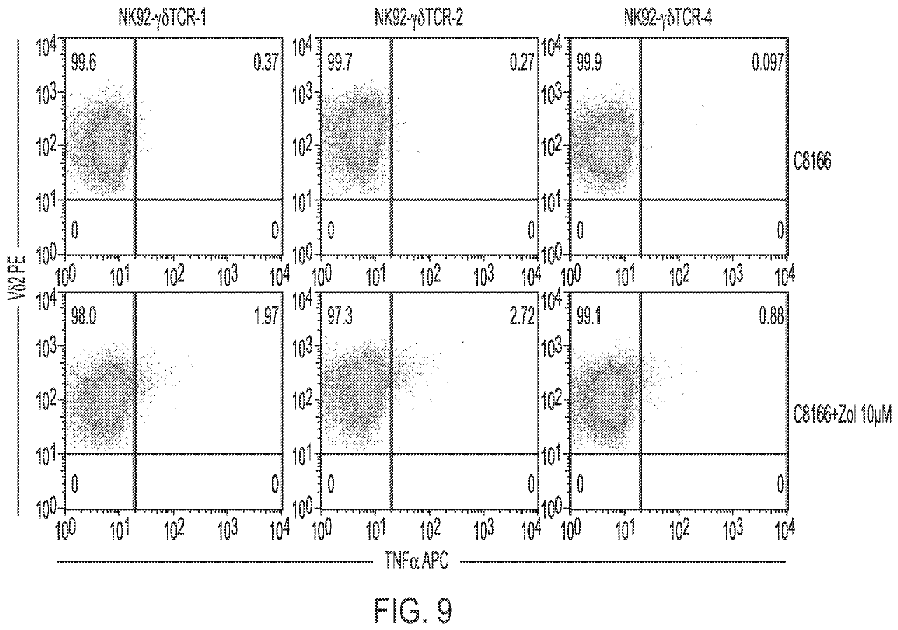

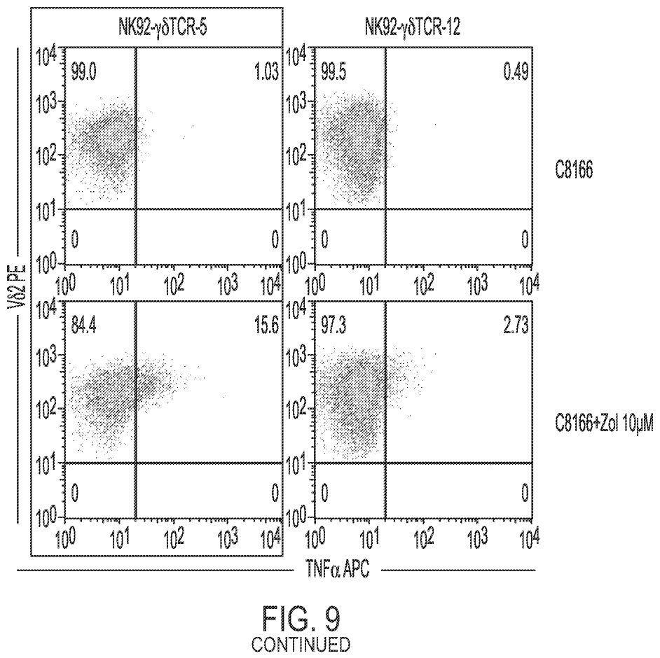

Similarly, as shown in FIG. 9, when TNFα was used as a marker, the NK92-γδTCR-5 subline had the highest level of expression. Specifically, when the NK92-γδTCR-5 subline was exposed to C8166 cells, 1.03% of the cells were positive for the TNFα marker (see FIG. 9). This percentage of TNFα positive cells increased to 15.6%, when the NK92-γδTCR subline was exposed to CD1866 cells that had been treated with zoledronic acid (see FIG. 9).

FIGS. 8 and 9 show that most of the cloned cell lines failed to express cell surface TCR and/or failed to demonstrate biological responses against tumor cell targets. Two cloned cell lines (the NK92-γδTCR5 subline and the NK92-γδTCR12 subline) were positive in all tests but still differed in potency.

One clone cell line, the NK92-gdTCR-5 subline, was selected for further study, as shown in the following Examples.

### Example 8: Exposure of Daudi Cells to the NK92-γδTCR-5 Subline Resulted in Cell Cytolysis

NK92 or NK92γδTCR-5 cells were tested to determine if the cells had cytotoxic effects on Daudi cells or zoledronic acid-treated Daudi cells.

Experimental Methods:

A fluorometric cytotoxicity assay with calcein-acetoxymethyl (Molecular Probes, Eugene, OK) was used to measure cytotoxicity against target cells. Daudi cells were treated without or with zoledronic acid (10 μM) for 24 hours and used as target cells. Target cells were labelled for 15 min with 2 mM calcein-acetoxymethyl at 37° C., then washed once with PBS. Cells were combined at various effector-to-target (E:T) ratios in 96-well round-bottom microtitre plates (Corning, NY) and incubated at 37° C. in 5% CO₂ for 4 hr; assays were performed in triplicate. After incubation, supernatants were transferred to a 96-well flat-bottom microtitre plate and calcein content was measured using a BioTek Synergy HT plate reader (485/535 nm). Percent specific lysis was calculated as: (test release−spontaneous release)/(maximum release−spontaneous release)×100.

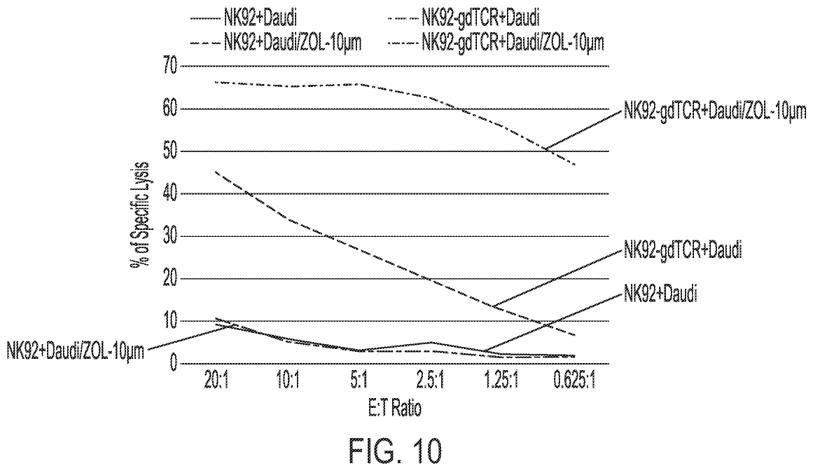

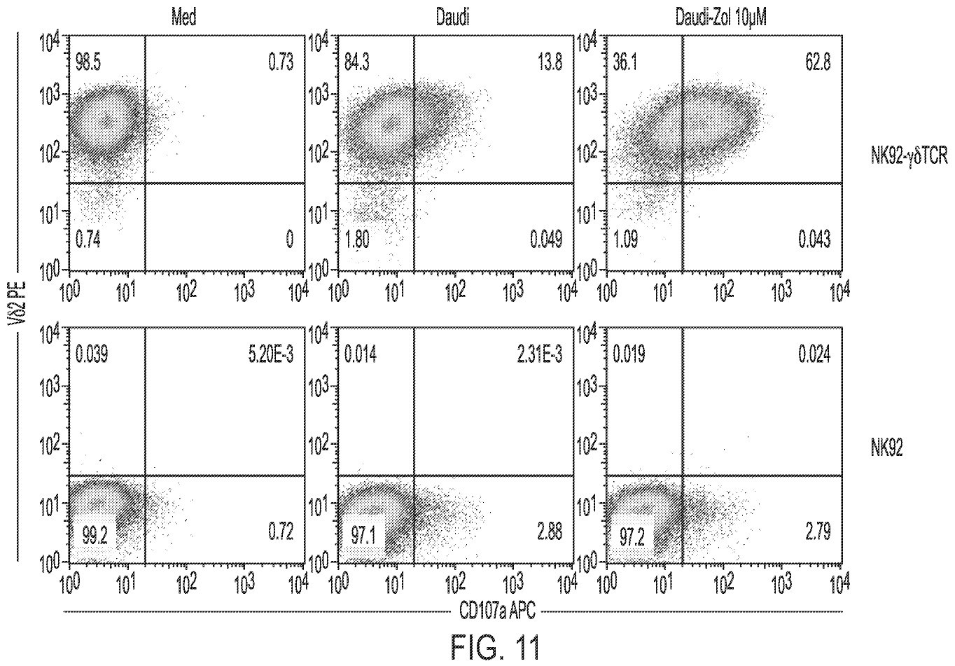

Several effector to target (E:T) ratios were tested, with triplicate wells for each condition. As shown in FIG. 10, NK92 showed similar low-level killing to Daudi and zoledronic acid-treated Daudi cells (see lines that correspond to NK92+Daudi and NK92+Daudi/ZOL-10 μM in FIG. 10). However, the NK92γδTCR-5 subline showed much higher cytotoxicity to Daudi cells compared with NK92 cells (see line that corresponds to NK92-gdTCR+Daudi in FIG. 10). At a 20:1 effector to target ratio, approximately 45% of the cells were lysed. Cell lysis dropped below 10% when the effector to target ratio was at 0.625:1. Zoledronic acid treatment further enhanced cell killing caused by the NK92γδTCR-5 subline (see line that corresponds to NK92-gdTCR+Daudi/ZOL-10 μM in FIG. 10). At a 20:1 effector to target ratio, over 65% of the cells were lysed (see FIG. 10). Surprisingly, a high proportion of zoledronic acid-treated Daudi cells were lysed by the NK92γδTCR-5 subline even at the at 2.5:1 E:T ratio, at a percent that was very similar to killing observed at 10:1 or even 20:1 ratio (see FIG. 10). Typically, for conventional NK or ye, T cell killing assays, it is common to use 20:1 or 10:1 E:T ratios to ensure a high enough potency for in vitro assays. Percent cell lysis dropped but was still near 50% even when the effector to target ratio was at 0.625:1 (see FIG. 10).

### Example 9: NK92 Cells Engineered with a γδ T Cell Receptor Produced a Degranulation Response when Exposed to Daudi Cells

NK92 or NK92γδTCR-5 were tested to determine whether they produced a degranulation response when exposed to Daudi or zoledronic acid-treated Daudi cells. Degranulation measures the Granzyme response and is a surrogate marker for cytotoxic cell activation. The degranulation response was evaluated using CD107a expression.

Experimental Methods:

Daudi cells were treated without or with zoledronic acid (10 μM) for 24 hours. NK92 or NK92γδTCR-5 were treated with medium, C8166 cells, or 10 μM zoledronic acid-treated C8166 cells at a 1:1 ratio for 4 hours. CD107a was measured by flow cytometry. Cells were washed, resuspended in 50-100 μl of staining buffer, and stained with mouse anti-human Vδ2-PE clone B6 and mouse anti-human CD107a-APC clone H4A3. Data for at least 1×10⁴ lymphocytes (gated on the basis of forward- and side-scatter profiles) were acquired from each sample on a FACSCalibur flow cytometer (BD Biosciences). All data were analyzed using FlowJo software.

As shown in FIG. 11, NK92 cells showed similar low-level response to Daudi and zoledronic acid-treated Daudi cells. However, NK92γδTCR-5 cells showed much higher response to Daudi cells compared with NK92 cells (see FIG. 11). Specifically, 13.8% of the NK92-γδTCR-5 cells expressed the CD107a marker when exposed to Daudi cells, compared to negligible expression of CD107a marker when NK92 cells were exposed to Daudi cells (see FIG. 11). Zoledronic acid treatment further enhanced the response of the NK92γδTCR-5 subline (see FIG. 11). Specifically, 62.8% of the NK92-γδTCR-5 cells expressed the CD107a marker when exposed to Daudi cells that had been treated with zoledronic acid, compared to negligible expression of the CD107a marker when NK92 cells were exposed to Daudi cells that had been treated with zoledronic acid (see FIG. 11).

### Example 10: NK92 Cells Engineered with a γδ T Cell Receptor Produced a TNFα Response when Exposed to Daudi Cells

NK92 or NK92γδTCR-5 were tested to determine whether they produced a cytokine response when exposed to Daudi or zoledronic acid-treated Daudi cells. The cytokine response was evaluated using intracellular TNFα expression. Production of the cytokine TNFα is a marker of cell activation. Here, the cytokine response reflects the strength of signal for NK92-γδTCR recognition of tumor cells.

Experimental Methods:

Daudi cells were treated without or with zoledronic acid (1004) for 24 hours. NK92 or NK92γδTCR-5 were treated with medium, C8166 cells, or 10 μM zoledronic acid-treated C8166 cells at a 1:1 ratio for 4 hours. TNFα was measured by flow cytometry. Cells were stained with mouse anti-human Vδ2-PE clone B6 then fixed, permeabilized, and incubated for 45 min at 4° C. with mouse anti-human TNF-α-APC. Intracellular staining solutions were obtained from the Cytofix/Cytoperm Kit (BD Biosciences). Cells were washed with staining buffer and resuspended. Data for at least 1×10⁴ lymphocytes (gated on the basis of forward- and side-scatter profiles) were acquired from each sample on a FACSCalibur flow cytometer (BD Biosciences). All data were analyzed using FlowJo software.

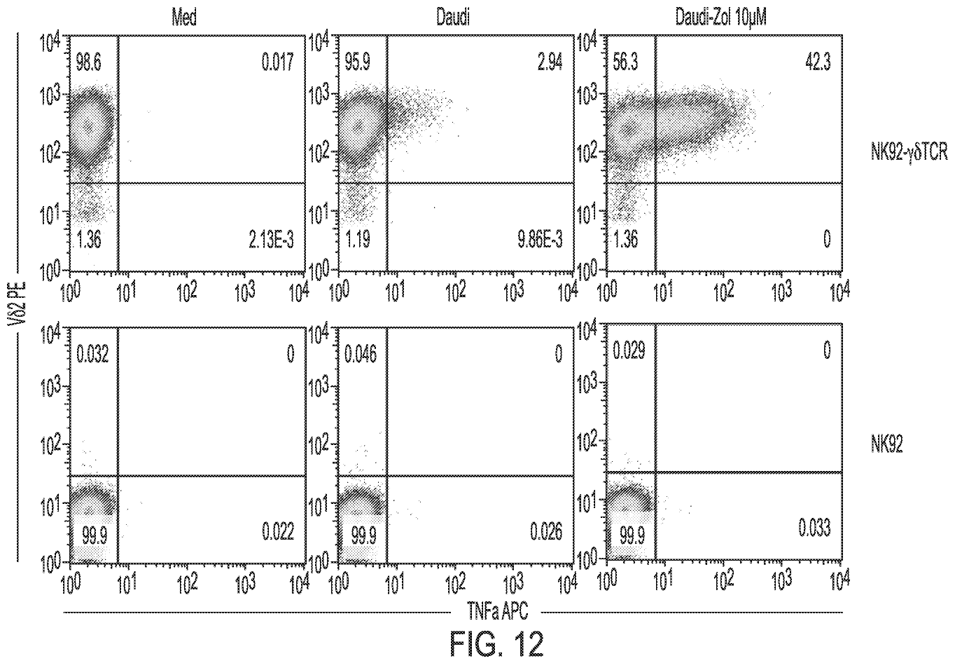

As shown in FIG. 12, NK92 cells showed similar low-level response to Daudi and zoledronic acid treated Daudi cells. However, the NK92γδTCR-5 subline showed much higher response to Daudi cells compared with NK92 cells (see FIG. 12). Specifically, 2.94% of the NK92-γδTCR-5 cells expressed the TNFα marker when exposed to Daudi cells, compared to negligible expression of TNFα marker when NK92 cells were exposed to Daudi cells (see FIG. 12). Zoledronic acid treatment further enhanced cell response by the NK92γδTCR-5 subline (see FIG. 12). Specifically, 42.3% of the NK92-γδTCR-5 cells expressed the TNFα marker when exposed to Daudi cells that had been treated with zoledronic acid, compared to negligible expression of TNFα marker when NK92 cells were exposed to Daudi cells that had been treated with zoledronic acid (see FIG. 12).

### Example 11: Exposure of TU167 Cells to the NK92-γδTCR-5 Subline Resulted in Cell Lysis

The NK92-γδTCR-5 subline was tested to determine whether it had cytotoxic effects on TU167 cells. TU167 cells (subline of the UM-SCC cell line) are a model for squamous cell carcinoma head and neck.

Experimental Methods:

A fluorometric cytotoxicity assay with calcein-acetoxymethyl (Molecular Probes, Eugene, OK) was used to measure cytotoxicity against target cells. TU167 cells were transduced without or with lentivirus vector expressing a shRNA that inhibits expression of farnesyl diphosphate synthase (AGT401; see FIG. 5) and treated without or with zoledronic acid (10 μM) for 24 hours and used as target cells. Target cells were labelled for 15 min with 2 mM calcein-acetoxymethyl at 37° C., then washed once with PBS. Cells were combined at various effector-to-target (E:T) ratios in 96-well round-bottom microtitre plates (Corning, NY) and incubated at 37° C. in 5% CO₂ for 4 hr; assays were performed in triplicate. After incubation, supernatants were transferred to a 96-well flat-bottom microtitre plate and calcein content was measured using a BioTek Synergy HT plate reader (485/535 nm). Percent specific lysis was calculated as: (test release−spontaneous release)/(maximum release−spontaneous release)×100.

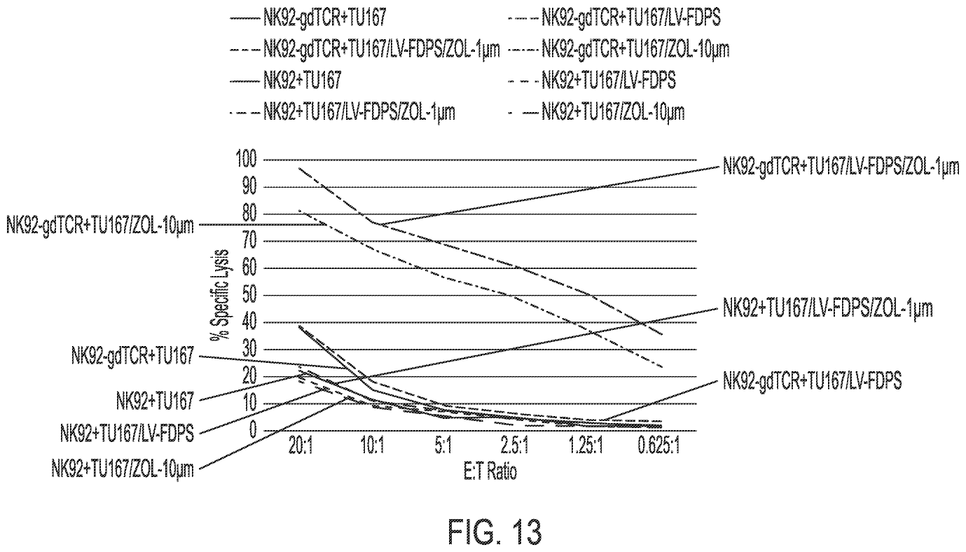

The cytotoxicity of NK92 or NK92γδTCR-5 against TU167 at different conditions was evaluated at several effector to target (E:T) ratios with triplicate wells for each condition. As shown in FIG. 13, NK92 showed similar low-level killing on TU167 at all conditions; notably FDPS knockdown by transduction with lentivirus vector AGT401 did not affect cell killing by NK92γδTCR-5 (see all lines in FIG. 13 that correspond to experimental conditions in which NK92 cells were used). However, NK92γδTCR-5 cells showed much higher cytotoxicity to TU167 cells compared with NK92 (see lines in FIG. 13 that correspond to experimental conditions in which NK92-gdTCR cells were used). Specifically, exposure of TU167 cells not treated with zoledronic acid to NK92γδTCR-5 cells resulted in just under 40% cell lysis of TU167 cells at 20:1 effector to cell ratio (see FIG. 13). AGT401 lentivirus vector (see FIG. 5) transduction combined with low concentration zoledronic acid (1 μM), or a higher concentration of zoledronic acid (10 μM) treatment significantly enhanced cell killing by the NK92γδTCR-5 subline (see lines that correspond to experimental conditions in which NK92-gdTCR cells were used along with ZOL-1 μm and ZOL-10 μm; FIG. 13). Specifically, exposure of TU167 cells treated with 1 μM zoledronic acid to NK92γδTCR-5 cells resulted in just under 100% cell lysis of TU167 cells at a 20:1 effector to cell ratio; cell lysis decreased to below 40% when the effector to cell ratio was reduced to 0.625:1 (see FIG. 13). Also, exposure of TU167 cells treated with 1004 zoledronic acid to NK92γδTCR-5 cells resulted in just over 80% cell lysis of TU167 cells at a 20:1 effector to cell ratio; cell lysis decreased to approximately 25% when the effector to cell ratio was reduced to 0.625:1 (see FIG. 13).

In sum, NK92γδTCR-5 showed much higher cytotoxicity against TU167 cells under various conditions. The LV-FDPS was designed to reduce the levels of farnesyl diphosphate synthase (FDPS) in cells. Reducing FDPS causes an accumulation of isopentenyl pyrophosphate (IPP) which is key to allowing γδ T cells to recognize and kill tumor cells. Zoledronic acid is a competitive inhibitor of FDPS. Thus LV-FDPS and Zoledronic acid both cause increases in IPP and enhanced TCR-dependent activation of NK92-γδTCR cell lines.

### Example 12: Exposure of Huh7 Cells to the NK92-γδTCR-5 Subline Resulted in Cell Lysis

The NK92-γδTCR-5 subline was tested to determine whether it had cytotoxic effects on Huh7 cells. Huh7 cells are a model for hepatocellular carcinoma.

Experimental Methods:

A fluorometric cytotoxicity assay with calcein-acetoxymethyl (Molecular Probes, Eugene, OK) was used to measure cytotoxicity against target cells. Huh7 cells were transduced without or with lentivirus vector AGT401 expressing miRNA capable of inhibiting FDPS protein production (SEQ ID NOs: 50, 51, 52, and 53) and treated without or with zoledronic acid (10 μM) for 24 hours and used as target cells. Target cells were labelled for 15 mM with 2 mM calcein-acetoxymethyl at 37° C., then washed once with PBS. Cells were combined at various effector-to-target (E:T) ratios in 96-well round-bottom microtitre plates (Corning, NY) and incubated at 37° C. in 5% CO₂ for 4 hr; assays were performed in triplicate. After incubation, supernatants were transferred to a 96-well flat-bottom microtitre plate and calcein content was measured using a BioTek Synergy HT plate reader (485/535 nm). Percent specific lysis was calculated as: (test release−spontaneous release)/(maximum release−spontaneous release)×100.

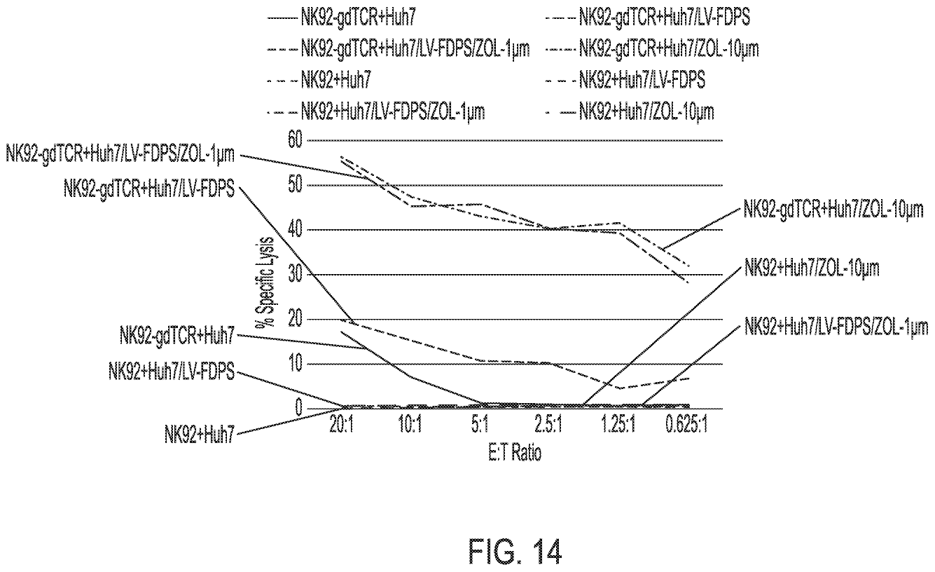

The cytotoxicity of NK92 or NK92γδTCR-5 against Huh7 under different conditions was evaluated at several effector to target (E:T) ratios with triplicate wells for each condition. As shown in FIG. 14, NK92 cells were unable to kill Huh7 under any of the conditions (see lines in FIG. 14 that correspond to the use of NK92 cells). NK92γδTCR-5 showed low level cytotoxicity on TU167 cells (see line that corresponds to NK92-gdTCR+Huh7 in FIG. 14). FDPS knockdown by transduction of lentivirus vector AGT401 (see FIG. 5) increased cell killing by NK92γδTCR-5 (see line that corresponds to NK92-gdTCR+Huh7/LV-FDPS in FIG. 14). Specifically, at an E:T ratio of 20:1 approximately 20% of the cells were lysed (FIG. 14). AGT401 lentivirus vector transduction combined with 1 μM concentration of zoledronic acid or 10 μM concentration of zoledronic acid significantly enhanced cell killing by NK92γδTCR-5 (see lines that correspond to treatment with NK92-gdTCR along with ZOL-1 μM and ZOL 10 μM in FIG. 14). Specifically, at both 1 μM and 10 μM concentrations of zoledronic acid, there was approximately 55% cell lysis of Huh7 cells at an effector to target ratio of 20:1; cell lysis was still around 30% even when the effector to target ratio was reduced to 0.625:1 (see FIG. 14).

### Example 13: Screening Single Cell Clones of NK92γδTCR Cells

NK92 cells were transduced with lentiviruses expressing Vγ9Vδ2 T cell receptor (TCR) or CD3 protein at moi 5. Single cells that express high-level of CD3 and Vγ9Vδ2 TCR were isolated with the BD cell sorting system (the BD FACSAria III Cell Sorter). The isolated single cells were incubated in 96-well plate at 37° C., 5% CO₂. After 3 to 4 weeks, cells were transferred to 24-well plate. Cell function was tested with Daudi or zoledronic Acid treated Daudi cells. Daudi is a target cell for the Vγ9Vδ2 TCR. If CD3 and Vγ9Vδ2 TCR in NK92 cells are functional, the Vγ9Vδ2 TCR positive NK92 will respond to Daudi cells and express CD107a on the cell surface as a marker of degranulation. Zoledronic acid blocks farnesyl diphosphate synthase (FDPS) leading to isopentenyl pyrophosphate (IPP) accumulation in cells; this enhances the response of Vγ9Vδ2 TCR positive NK92 cells.

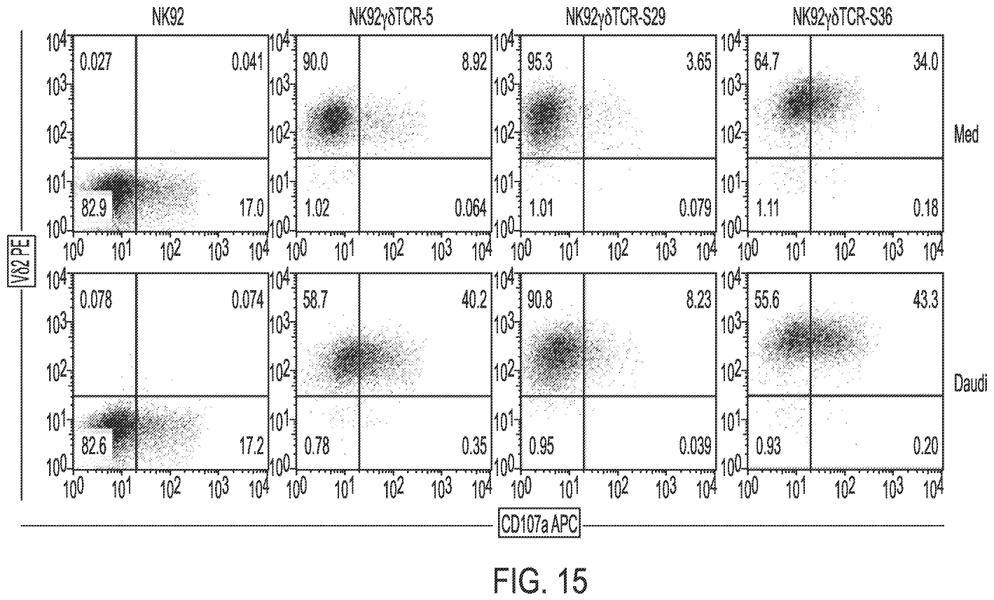

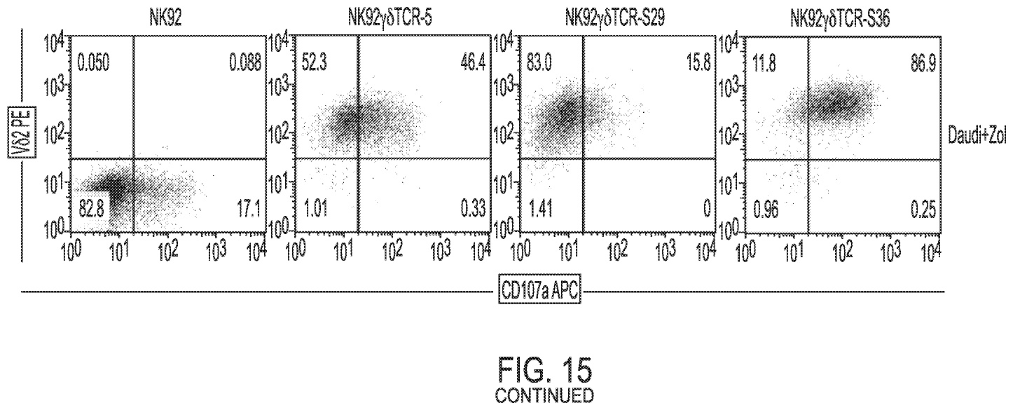

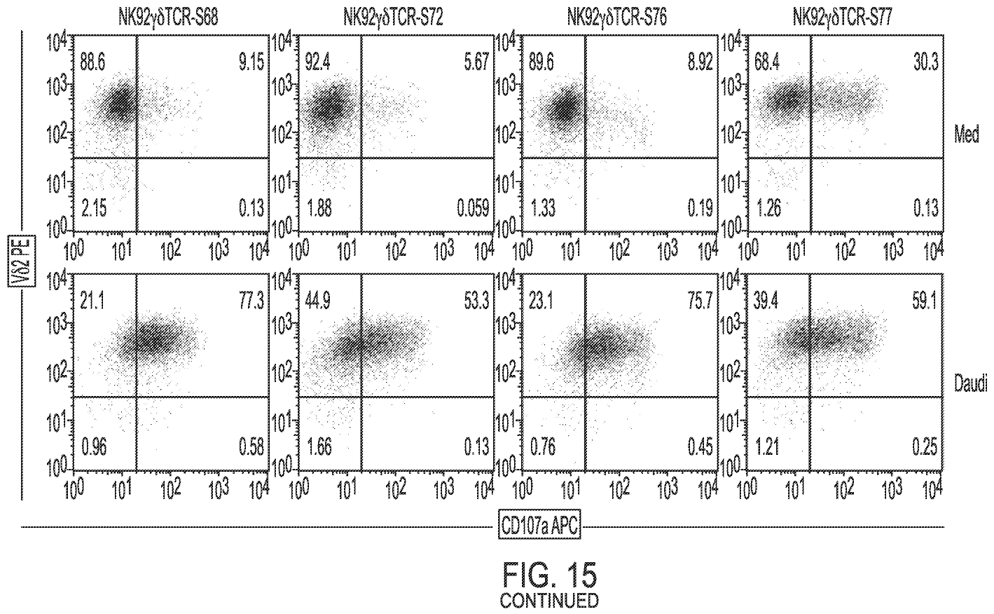

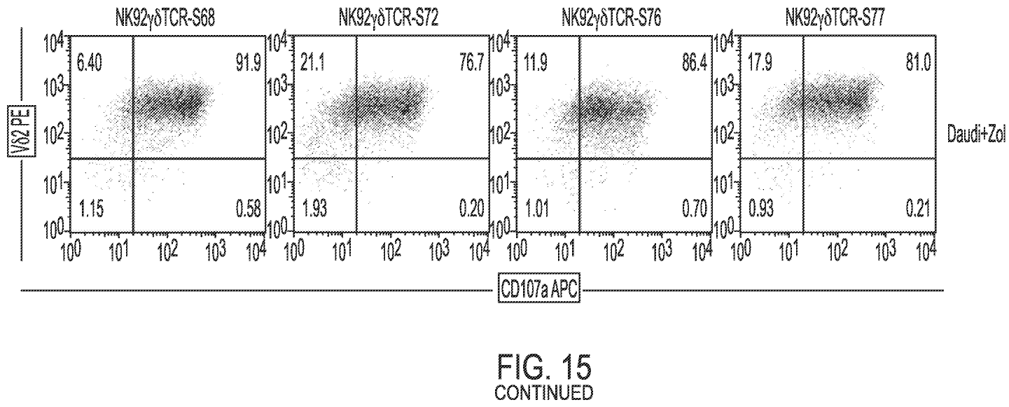

Daudi cells were treated without or with zoledronic acid (10 μM) for 24 hours. NK92 or NK92γδTCR single cell clones were treated with Medium, Daudi, or 10 μM zoledronic acid treated Daudi cells at 1:1 ratio for 4 hours. CD107a was measured by flow cytometry (FIG. 15). Cells were washed, resuspended in 50-100 μl of staining buffer, and stained with mouse anti-human Vδ2-PE clone B6 and mouse anti-human CD107a-APC clone H4A3. Data for at least 1×10⁴ lymphocytes (gated on the basis of forward- and side-scatter profiles) were acquired from each sample on a FACSCalibur flow cytometer (BD Biosciences). All samples were analyzed using FlowJo software.

More than 100 single cell clones were screened by single cell sorting. Five (5) cell clones showed strong responses. As shown in FIG. 15, NK92 cells showed similar low-level response on Daudi and zoledronic acid-treated Daudi cells (see FIG. 15, first column of data, showing that 0.074% and 0.088% of NK92 cells were positive for CD107a in Daudi cells and zoledronic acid-treated Daudi cells, respectively). However, some single cell clones of NK92γδTCR showed a higher response on Daudi cells compared with NK92 cells (see, middle row of columns 2-8 of FIG. 15, representing different screened sublines, showing that 40.2%, 8.23%, 43.3%, 77.3%, 53.3%, 75.7%, and 59.1%, respectively, of cells expressing CD107a). Zoledronic acid treatment further enhanced cell response by the NK92γδTCR cloned cell lines (see, bottom row columns 2-8 of FIG. 15, representing different screened sublines, showing that 46.4%, 15.8%, 86.9%, 91.9%, 76.7%, 86.4%, and 81.0%, respectively, of cells expressing CD107a). NK92γδTCR-S29 is a representative cell clone that did not show a strong response, with 8.23% and 15.8% of cells positive for CD107a after treatment with Daudi cells and zoledronic acid-treated Daudi cells, respectively (see middle and lower rows of column 3 of FIG. 15).

Data were compiled for NK92γδTCR cloned cell lines obtained by limiting concentration dilution (see method described in Example 6) or by flow cytometry single cell sorting. The percentage of CD107a positive cells is depicted for various NK92γδTCR cloned cell lines after exposure to tumor cells or exposure to tumor cells plus zoledronic acid (see Table 1). The range of values demonstrates substantial heterogeneity among cloned cell lines. The ratio of CD017a positive cells for tumor+zoledronic acid divided by tumor alone is one means of describing the specificity of tumor cell recognition. An ideal NK92γδTCR effector cell will demonstrate a potent response to tumor cells plus zoledronic acid (above 65% positive) and demonstrate a substantial increase in response for tumor plus zoledronic acid versus tumor alone. Four cloned cell lines, indicated by arrows, demonstrate potent responses and specificity for tumor cells treated with zoledronic acid. These cloned cell lines are preferred choices for clinical application in cancer immunotherapy.

TABLE 1

| | | Percentage | Ratio of % CD017a |
|---|---|---|---|
| | Percentage | CD017a | positive:(Tumor |
| Cloned Cell | CD017a | positive with | cells plus |
| Line ID | positive with | tumor cells plus | Zoledronic acid) ÷ |
| Number | tumor cells | zoledronic acid | (Tumor cells) |
| 1 | 2.9 | 5.7 | 2.0 |
| 2 | 1.4 | 3.9 | 2.8 |
| 4 | 5.3 | 28.7 | 5.4 |
| [symbol] 5 | 17.8 | 68.2 | 3.8 |
| 12 | 5.7 | 25.7 | 4.5 |
| S29 | 8.2 | 15.8 | 1.9 |
| [symbol] S36 | 43.3 | 86.9 | 2.0 |
| S68 | 77.3 | 91.9 | 1.2 |
| [symbol] S72 | 53.3 | 76.7 | 1.4 |
| S76 | 75.7 | 86.4 | 1.1 |
| [symbol] S77 | 59.1 | 81.0 | 1.4 |

Table 1 presents data for 11 cloned cell lines showing the percentages of cloned cells positive for CD107a after exposure to tumor cells or exposure to tumor cells plus Zoledronic Acid. The relative increase after including Zoledronic Acid is shown.

Cloned NK92 cell lines 5, S36, S72 and S77 were selected for further development. The criteria for selecting these clones were: cloned cell lines expressing the Vγ9Vδ2 T cell receptor having a percentage CD107a positive cells >65 using tumor plus zoledronic acid and a ratio (Table 1 column 4)>1.2.

### Example 14: Screening of Sorted NK92 Vγ9Vδ2 TCR Cell Clones According to their Response Against C8166 Cells

Specific cloned NK92 cell lines from the previous example were screened according to their response against C8166 Cells. The C8166 cell line was derived from a T cell leukemia. The selected cloned cell lines were treated with C8166 cells at 1:1 ratio for 4 hours. CD107a was measured by flow cytometry. For detecting CD107a, cells were washed, resuspended in 50-100 μl of staining buffer, and stained with mouse anti-human Vδ2-PE clone B6 and mouse anti-human CD107a-APC clone H4A3. Cells were washed with staining buffer and resuspended. Data for at least 1×10⁴ lymphocytes (gated on the basis of forward- and side-scatter profiles) were acquired from each sample on a FACSCalibur flow cytometer (BD Biosciences). All samples were analyzed using FlowJo software.

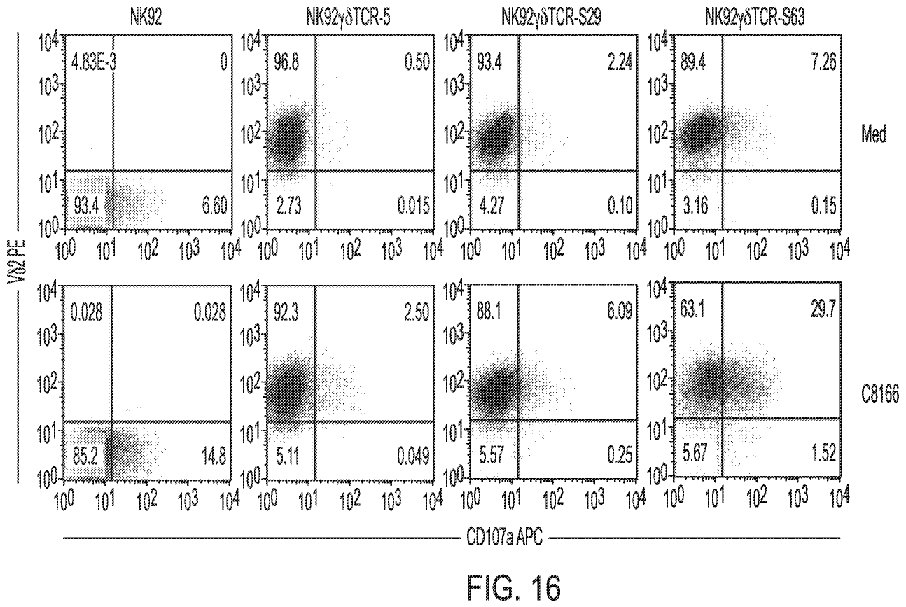

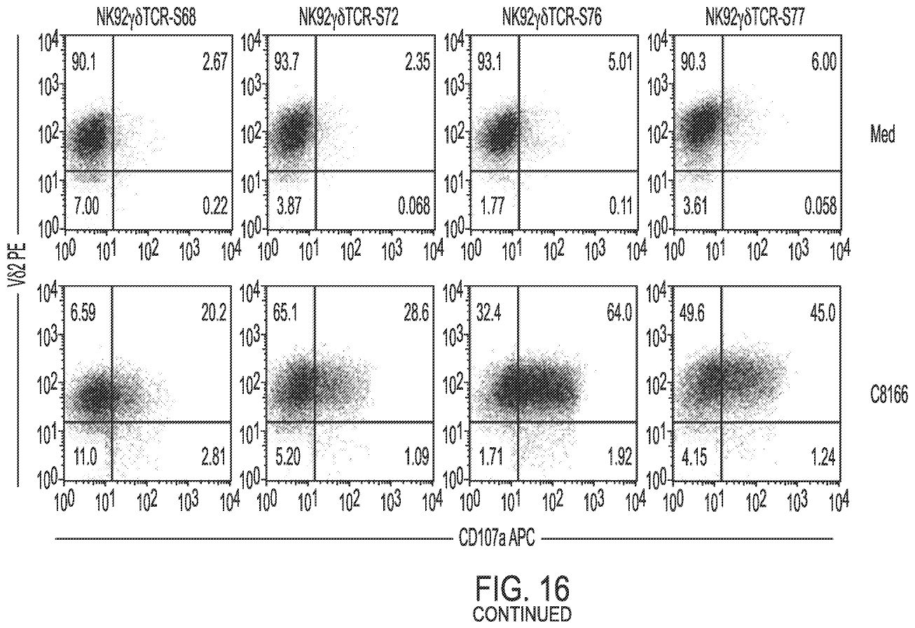

As shown in FIG. 16, cloned cell lines S63, S68, S72, S76 and S77 showed better response to C8166 cells. Among these clones, clone S76 showed the best response to C8166 cells.

### Example 15: Screening of Sorted NK92 Vγ9Vδ2 T Cell Receptor Clones by Response to More Cell Lines (TU167, Hela, MDA-MB-231 and A549)

Cloned NK92 cell lines described in the previous examples were screened for their response to TU167, Hela, MDA-MB-231 and A549 Cell lines. HeLa is a model for cervical carcinoma related to human papilloma virus infection. MDA-M B-231 is a cell line model for triple negative breast cancer and A549 is a model for lung adenocarcinoma.

The selected NK92 cell clones were treated with different cells at 1:1 ratio for 4 hours. CD107a was measured by flow cytometry. For detecting CD107a, cells were washed, resuspended in 50-100 μl of staining buffer, and stained with mouse anti-human Vδ2-PE clone B6 and mouse anti-human CD107a-APC clone H4A3. Cells were washed with staining buffer and resuspended. Data for at least 1×10⁴ lymphocytes (gated on the basis of forward- and side-scatter profiles) were acquired from each sample on a FACSCalibur flow cytometer (BD Biosciences). All samples were analyzed using FlowJo software.

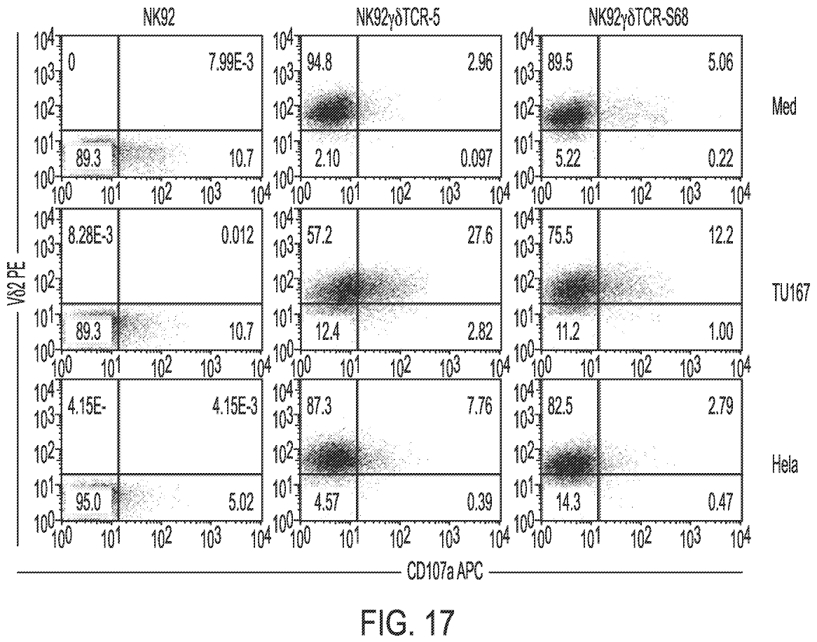

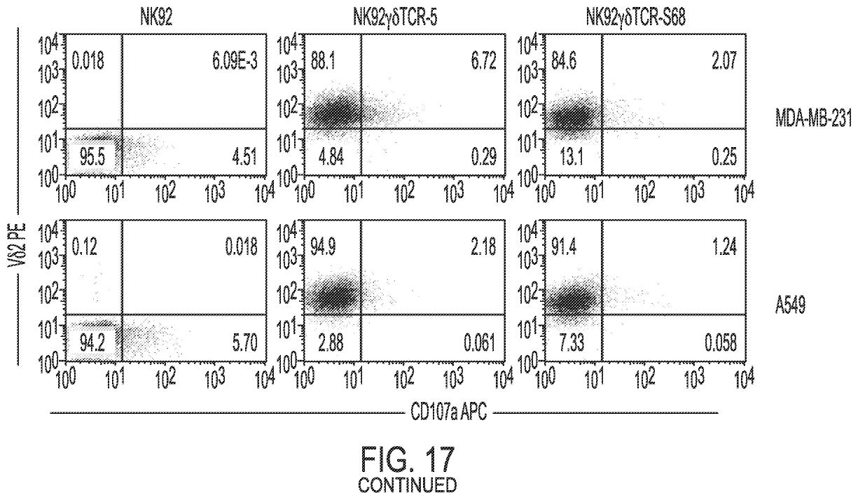

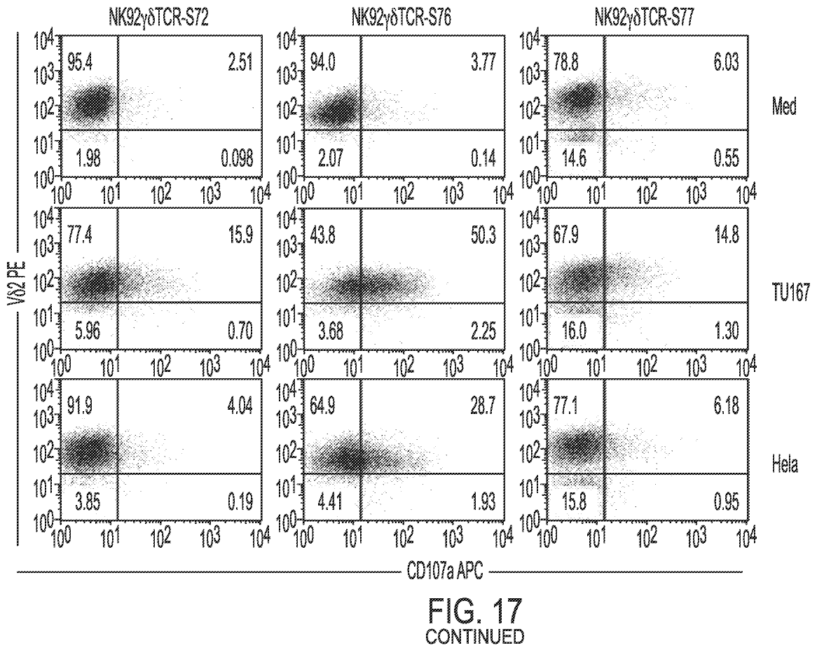

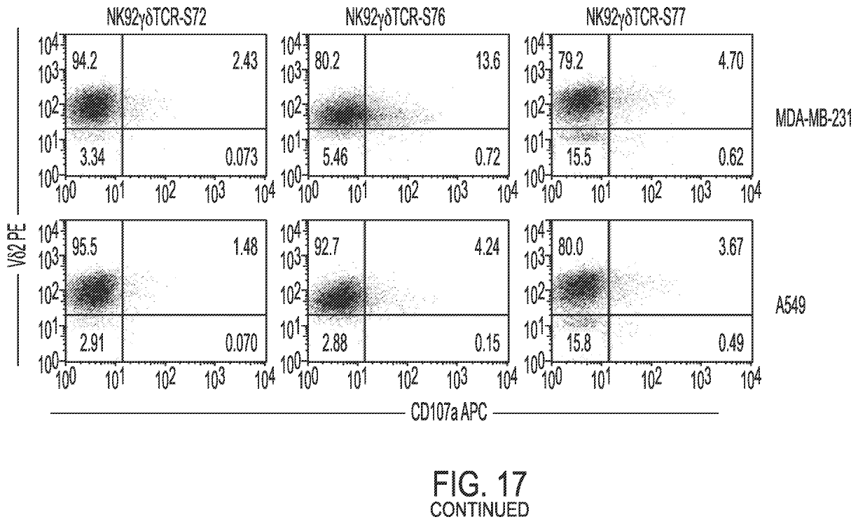

As shown in FIG. 17, among all cloned NK92 cells lines, clone S76 showed the best response overall to cell lines tested.

### Example 16: Screening of Sorted NK92 Vγ9Vδ2 T Cell Receptor Clones by Response to Huh7 or Zoledronic Acid (1 μM)-Treated Huh7 Cells

Cloned NK92 Cloned NK92 cell lines described in the previous examples were screened for their response against Huh7 or Zoledronic Acid (1 μM)-Treated Huh7 Cells.

Huh7 cells were treated without or with Zoledronic Acid (1 μM) for 24 hours. The sorted NK92 cell clones were treated with Huh7, or 10 μM Zoledronic acid treated Huh7 cells at 1:1 ratio for 4 hours. CD107a was measured by flow cytometry. For detecting CD107a, cells were washed, resuspended in 50-100 μl of staining buffer, and stained with mouse anti-human Vδ2-PE clone B6 and mouse anti-human CD107a-APC clone H4A3. Cells were washed with staining buffer and resuspended. Data for at least 1×10⁴ lymphocytes (gated on the basis of forward- and side-scatter profiles) were acquired from each sample on a FACSCalibur flow cytometer (BD Biosciences). All samples were analyzed using FlowJo software.

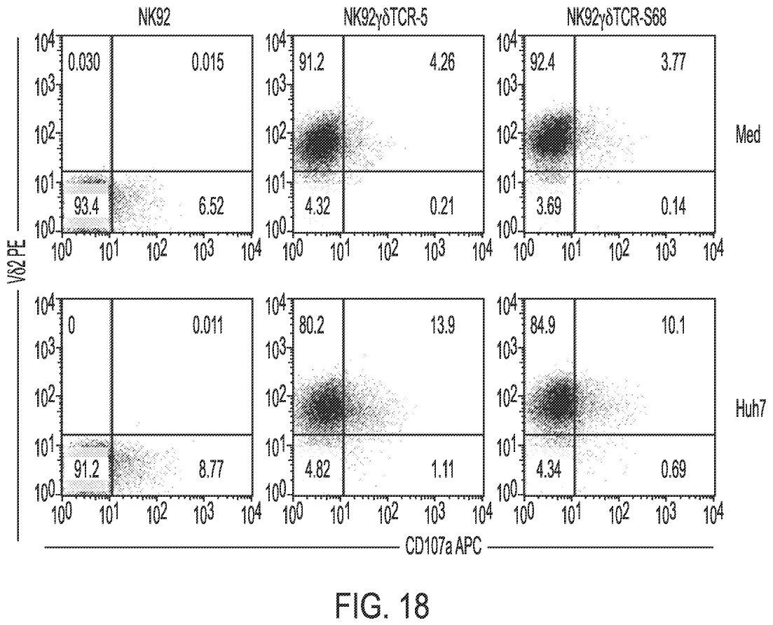

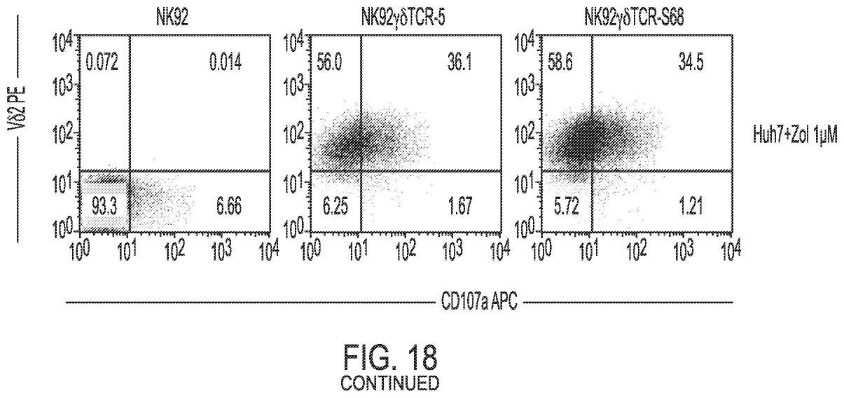

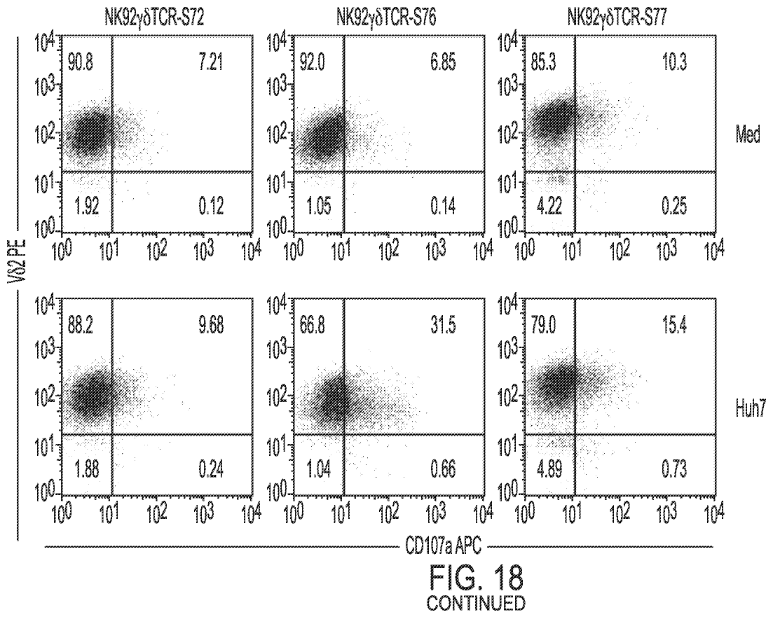

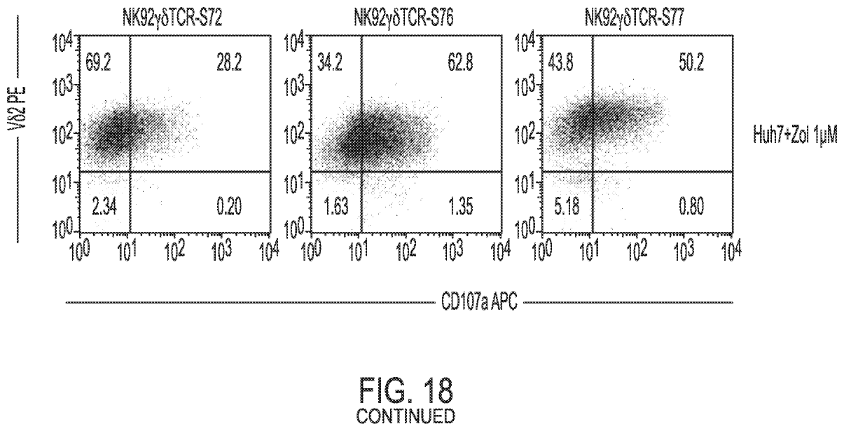

As shown in FIG. 18, clone S76 showed the best response to Huh7 or Zoledronic Acid-treated Huh7 cells.

### Example 17: Comparing the Response of S76 and S77 to Several Different Cancer Cell Lines

The cloned NK92 cell lines S76 and S77 were compared based on their responses to several cell lines. Jurkat is a model for T cell leukemia and J1.1 cells were derived from Jurkat after HIV infection. K562 is an erythroid leukemia cell line. SupT1 is a T cell lymphoma cell line and THP1 is a monocytic leukemia cell line.

The cloned NK92 cell lines S76 and S77 were mixed with different cancer cell lines at an E:T ratio of 1:1 for 4 hours. CD107a was measured by flow cytometry. For detecting CD107a, cells were washed, resuspended in 50-100 μl of staining buffer, and stained with mouse anti-human Vδ2-PE clone B6 and mouse anti-human CD107a-APC clone H4A3. Cells were washed with staining buffer and resuspended. Data for at least 1×10⁴ lymphocytes (gated on the basis of forward- and side-scatter profiles) were acquired from each sample on a FACSCalibur flow cytometer (BD Biosciences). All samples were analyzed using FlowJo software.

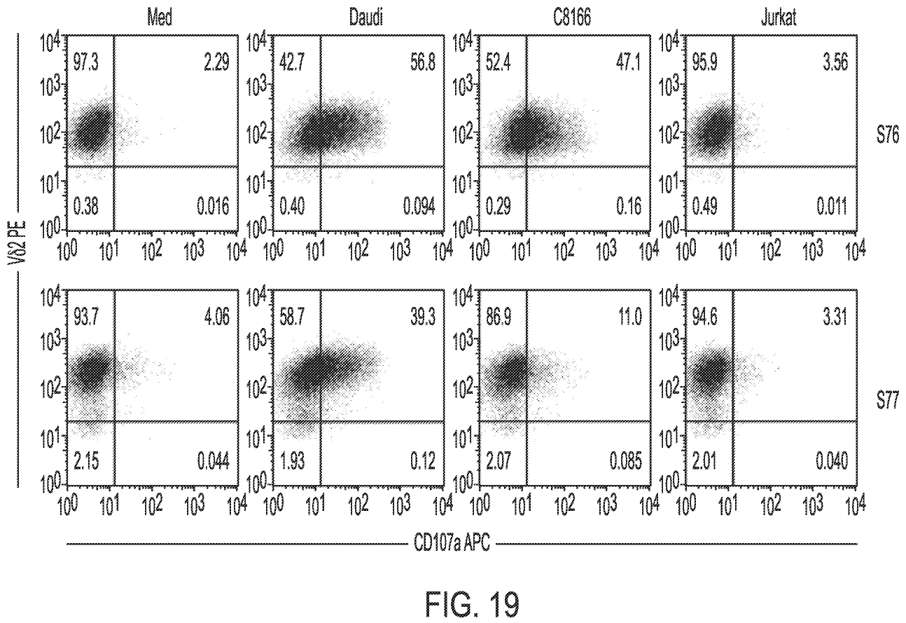

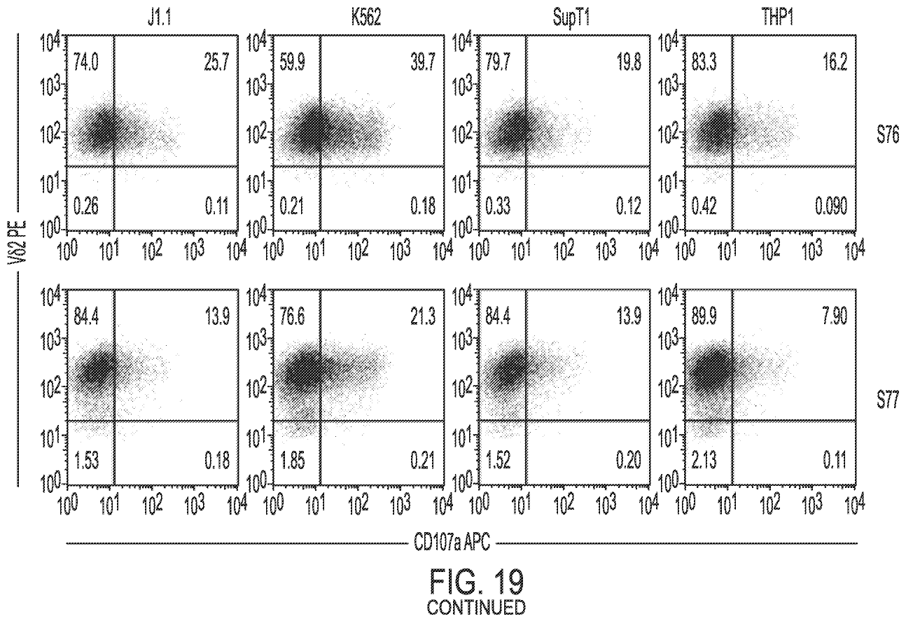

As shown in FIG. 19, clone S76 responded better than S77 to the multiple cancer cell lines tested.

### Example 18: Testing the Response of S76 to Multiple Cell Lines

The response of the S76 cell line was tested against multiple cancer cell lines after treatment with Zoledronic acid. PC3 is a prostate cancer cell subline obtained from the University of Maryland, Baltimore. PC3-ATCC was obtained from the American Type Culture Collection.

The S76 cell line was mixed with any of several cancer cell lines at a 1:1 ratio for 4 hours. CD107a was measured by flow cytometry. For detecting CD107a, cells were washed, resuspended in 50-100 μl of staining buffer, and stained with mouse anti-human Vδ2-PE clone B6 and mouse anti-human CD107a-APC clone H4A3. Cells were washed with staining buffer and resuspended. Data for at least 1×10⁴ lymphocytes (gated on the basis of forward- and side-scatter profiles) were acquired from each sample on a FACSCalibur flow cytometer (BD Biosciences). All samples were analyzed using FlowJo software.

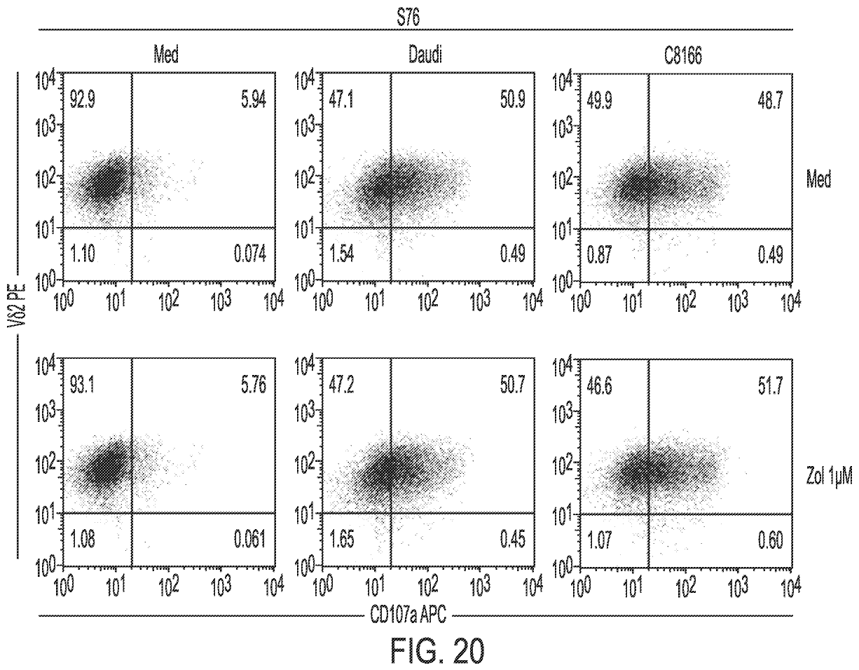

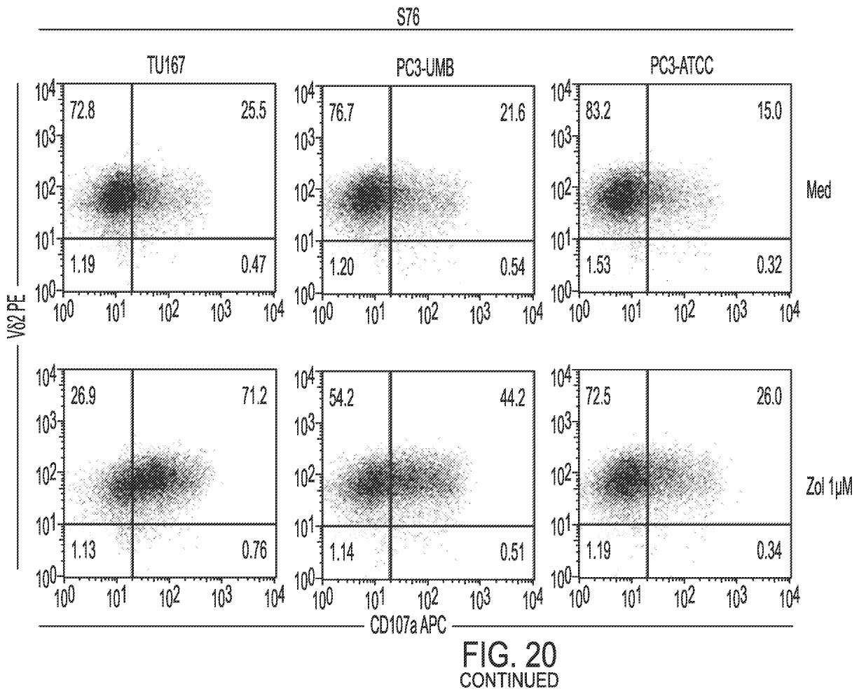

As shown in FIG. 20, the S76 cell line showed a response to all tested cell lines and the response was usually higher after low dose Zoledronic acid. Zoledronic acid had little effect on the response to Daudi or C8166 cells, these being the only T cell targets tested in this study.

### Example 19: Testing the Effect of Mitomycin C on the Response of the S76 Cell Line to Daudi Cell Line

Cloned S76 cells were inactivated to prevent proliferation before injecting cells into a cancer patient. Mitomycin C (“MMC”) was used to mimic irradiation and test whether the cells remain functional after MMC treatment. Irradiation may also be used.

Cloned S76 cells were treated with MMC at various concentration for 1 or 2 hours. After MMC treatment, S76 was mixed with Daudi cells at a 2:1 ratio for 4 hours. CD107a was measured by flow cytometry. For detecting CD107a, cells were washed, resuspended in 50-100 μl of staining buffer, and stained with mouse anti-human Vδ2-PE clone B6 and mouse anti-human CD107a-APC clone H4A3. Cells were washed with staining buffer and resuspended. Data for at least 1×104 lymphocytes (gated on the basis of forward- and side-scatter profiles) were acquired from each sample on a FACSCalibur flow cytometer (BD Biosciences). All samples were analyzed using FlowJo software.

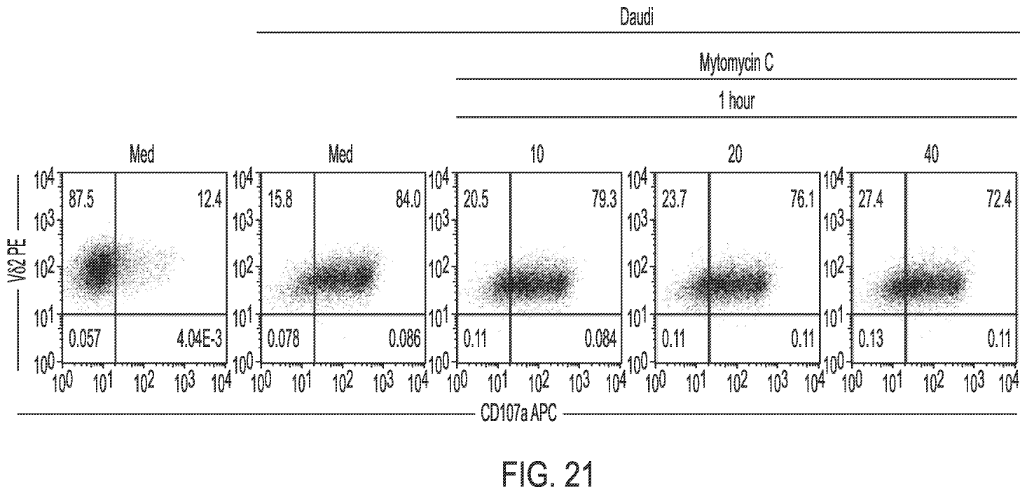

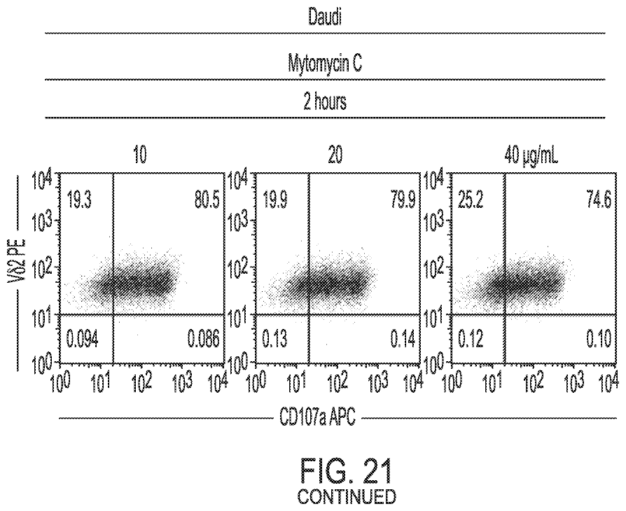

As shown in FIG. 21, MMC treatment did not significantly impair the function of the clone S76 cells even after 2 hours treatment with the highest dose tested here.

### Example 20: Determining the Cytotoxicity of NK92γδTCR-S76 Against C8166 Cells

Clone S76 was selected as a candidate for further development because it showed the best responses to several cell lines base on CD107a expression. We next wanted to confirm the results using a cytotoxicity assay. We compared the cloned S76 cell line (also referred to herein as “NK92γδTCR-S76”) and the NK92γδTCR-5 cell line in this assay.

A fluorometric cytotoxicity assay with calcein-acetoxymethyl (Molecular Probes, Eugene, OR) was used to measure cytotoxicity against target cells. Target cell lines treated without or with Zoledronic Acid (1 μM) for 24 hours and used as target cells. Target cells were labelled for 15 min with 2 mm calcein-acetoxymethyl at 37°, then washed once with PBS. Cells were combined at various effector-to-target (E:T) ratios in 96-well, round-bottomed microtiter plates (Corning, NY) and incubated at 37° in 5% CO2 for 4 hr; assays were performed in triplicate. After incubation, supernatants were transferred to a 96-well flat-bottomed microtiter plate and calcein content was measured using a Wallac Victor2 1420 multi-channel counter (485/535 nm). Percent specific lysis was calculated as: (test release−spontaneous release)/(maximum release−spontaneous release)×100.

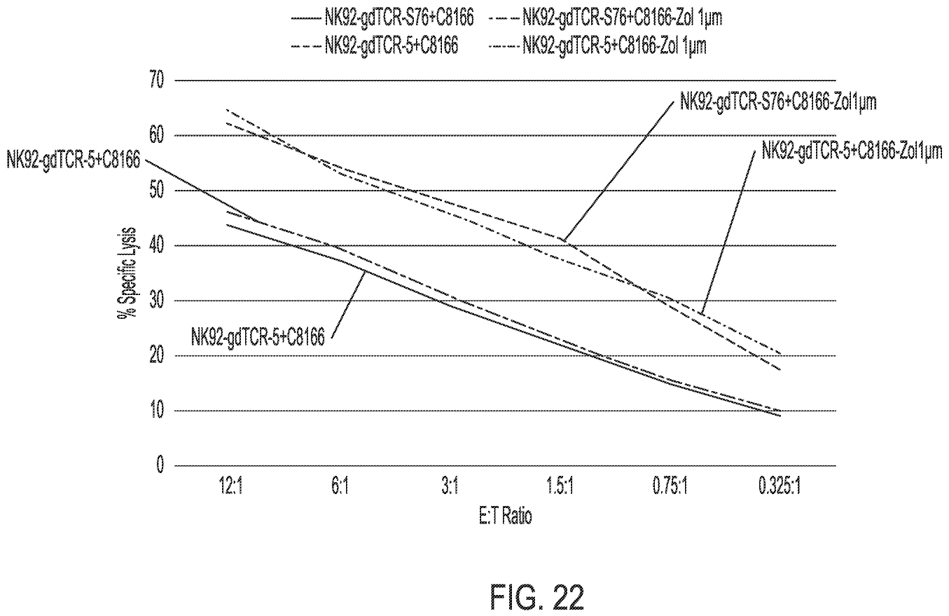

As shown in FIG. 22, NK92γδTCR-S76 showed higher cytotoxicity compared to NK92γδTCR-5 on C8166 cells or Zoledronic Acid (1 μM) treated C8166 cells. 1 μM concentration of Zoledronic acid did not change cell killing by NK92γδTCR-S76 or NK92γδTCR-5. These are consistent with previous CD107a assays.

### Example 21: Determining the Cytotoxicity of NK92γδTCR-S76 Against SNU447 Cell Line

It was determined whether NK92γδTCR-S76 has cytotoxic effects on SNU447 cell line. SNU447 is a leukemia cell line and cells were transformed by Epstein Barr virus.

A fluorometric cytotoxicity assay with calcein-acetoxymethyl (Molecular Probes, Eugene, OR) was used to measure cytotoxicity against target cells. Target cell lines treated without or with Zoledronic Acid (1 μM) for 24 hours and used as target cells. Target cells were labelled for 15 min with 2 mm calcein-acetoxymethyl at 37°, then washed once with PBS. Cells were combined at various effector-to-target (E:T) ratios in 96-well, round-bottomed microtiter plates (Corning, NY) and incubated at 37° in 5% CO2 for 4 hr; assays were performed in triplicate. After incubation, supernatants were transferred to a 96-well flat-bottomed microtiter plate and calcein content was measured using a Wallac Victor2 1420 multi-channel counter (485/535 nm). Percent specific lysis was calculated as: (test release−spontaneous release)/(maximum release−spontaneous release)×100.

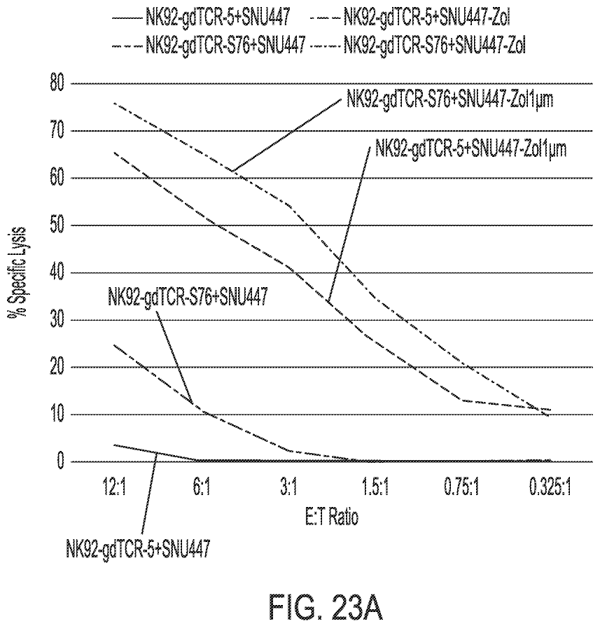

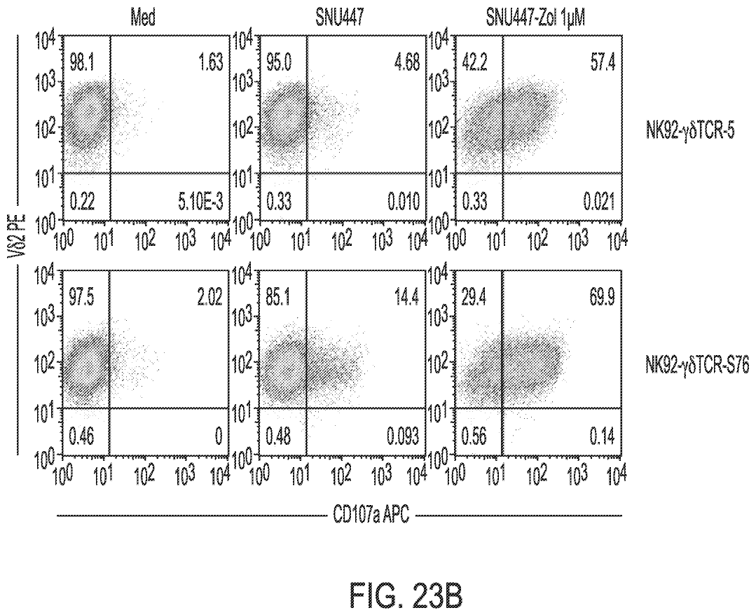

As shown in FIG. 23A, NK92γδTCR-576 showed higher level cytotoxicity than NK92γδTCR-5 on SNU447 cells or Zoledronic Acid (1 μM) treated SNU447 cells. 1 μM concentration of Zoledronic acid significantly enhanced cell killing by NK92γδTCR-576 or NK92γδTCR-5. These are consistent with CD107a assay (FIG. 23B).

### Example 22: Determining the Cytotoxicity of NK92γδTCR-S76 Against A549 Cell Line

It was determined whether NK92γδTCR-S76 has cytotoxic effects on A549 cell line. The A549 cell line is a model for lung adenocarcinoma.

A fluorometric cytotoxicity assay with calcein-acetoxymethyl (Molecular Probes, Eugene, OR) was used to measure cytotoxicity against target cells. Target cell lines treated without or with Zoledronic Acid (1 μM) for 24 hours and used as target cells. Target cells were labelled for 15 min with 2 mm calcein-acetoxymethyl at 37°, then washed once with PBS. Cells were combined at various effector-to-target (E:T) ratios in 96-well, round-bottomed microtiter plates (Corning, NY) and incubated at 37° in 5% CO2 for 4 hr; assays were performed in triplicate. After incubation, supernatants were transferred to a 96-well flat-bottomed microtiter plate and calcein content was measured using a Wallac Victor2 1420 multi-channel counter (485/535 nm). Percent specific lysis was calculated as: (test release−spontaneous release)/(maximum release−spontaneous release)×100.

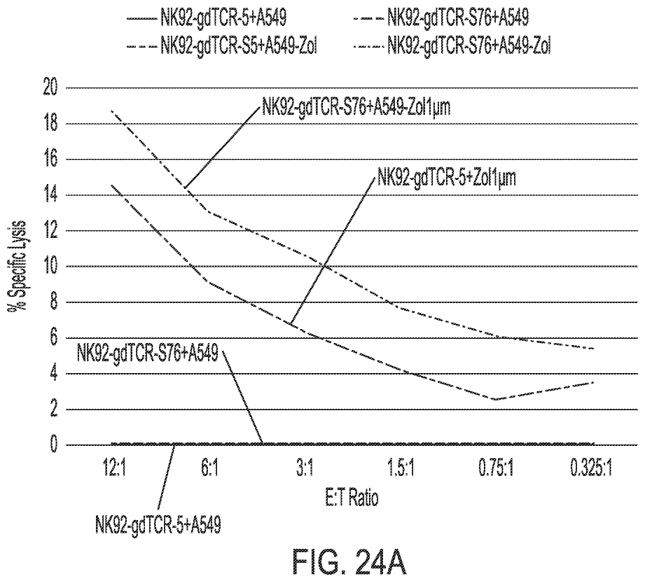

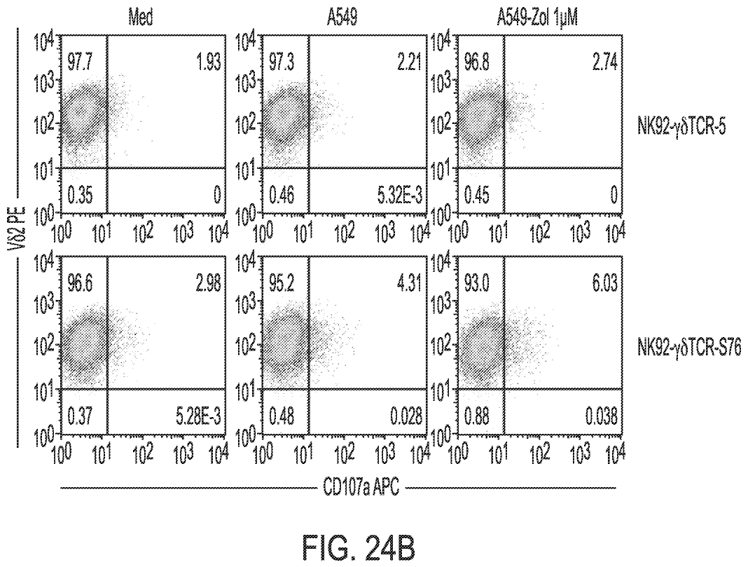

As shown in FIG. 24A, A549 is resistant to NK92γδTCR-576 and NK92γδTCR-5. 1 μM concentration of Zoledronic acid enhanced cell killing by NK92γδTCR-576 or NK92γδTCR-5. NK92γδTCR-576 showed higher level cytotoxicity than NK92γδTCR-5 on Zoledronic Acid (1 μM) treated A549 cells. These are consistent with results from a CD107a assay (FIG. 24B).

These results demonstrate that A549 is resistant to NK92γδTCR-576 and NK92γδTCR-5. Low level cell killing was observed when A549 was treated with Zoledronic Acid (1 μM). NK92γδTCR-576 showed higher cytotoxic effect cells than NK92γδTCR-5 on Zoledronic acid (1 μM) treated A549. In subsequent experiments (not shown) a 10 μM dose of Zoledronic acid substantially increased A549 cell killing by the NK92γδTCR-576 cloned effector cell line.

### Example 23: Determining the Cytotoxicity of NK92γδTCR-S76 Against PC3 Cell Line

It was determined whether NK92γδTCR-576 is cytotoxic against PC3 (UMB) cells. A fluorometric cytotoxicity assay with calcein-acetoxymethyl (Molecular Probes, Eugene, OR) was used to measure cytotoxicity against target cells. Target cell lines treated without or with Zoledronic Acid (1 μM) for 24 hours and used as target cells. Target cells were labelled for 15 min with 2 mm calcein-acetoxymethyl at 37°, then washed once with PBS. Cells were combined at various effector-to-target (E:T) ratios in 96-well, round-bottomed microtiter plates (Corning, NY) and incubated at 37° in 5% CO2 for 4 hr; assays were performed in triplicate. After incubation, supernatants were transferred to a 96-well flat-bottomed microtiter plate and calcein content was measured using a Wallac Victor2 1420 multi-channel counter (485/535 nm). Percent specific lysis was calculated as: (test release−spontaneous release)/(maximum release−spontaneous release)×100.

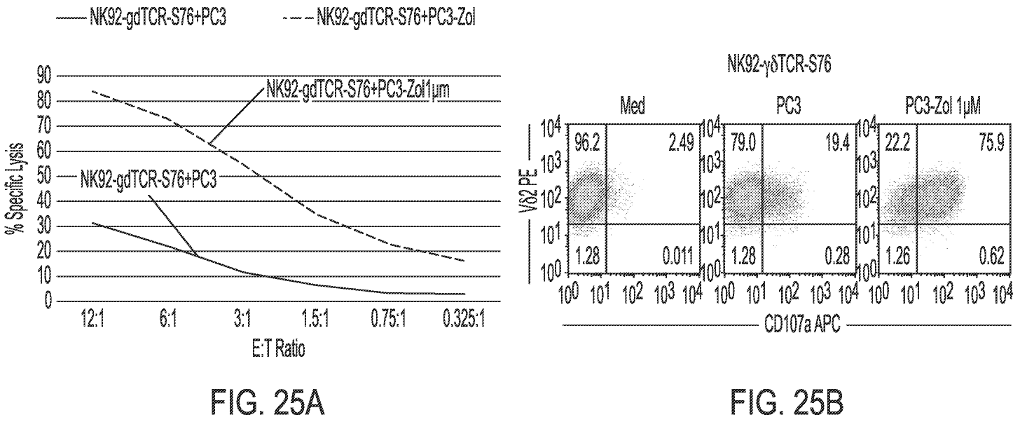

As shown in FIG. 25A, NK92γδTCR-576 showed low level on PC3 cells. 1 μM concentration of Zoledronic acid significantly enhanced cell killing by NK92γδTCR-576. These are consistent with previous CD107a assay results (FIG. 25B).

### Example 24: Phenotypic Characterization of Selected NK92γδTCR Cell Clones

As described above, by screening single cell clones, we selected clones that showed high response to Daudi or other cancer cell lines. Among these clones, NK92γδTCR-576 and NK92γδTCR-5 showed the best function. Further, NK92γδTCR-576 was better than NK92γδTCR-5 in multiple tests. To understand why different clones showed different effector functions, phenotype analysis was used to compare the expression of NK-related receptors on NK92, NK92γδTCR-576 and NK92γδTCR-5 cells.

The expression of NK-related receptors on NK92, NK92γδTCR-576 and NK92γδTCR-5 cells were compared. Mechanistic explanations for differences in effector cell potency were explored. NK92, NK92γδTCR-576 or NK92γδTCR-5 cells were washed, resuspended in 50-100 μl of staining buffer, and stained with different antibodies. Cells were washed with staining buffer and resuspended. Data for at least 1×10⁴ lymphocytes (gated on the basis of forward- and side-scatter profiles) were acquired from each sample on a FACSCalibur flow cytometer (BD Biosciences). All samples were analyzed using FlowJo software. The relative levels of NK activating receptor expression were determined by mean fluorescence intensity, with the results as follows:
NKp30: NK92γδTCR-576=NK92γδTCR-5>NK92
NKp44: NK92γδTCR-576>NK92γδTCR-5>NK92
NKp46 and NKG2D: Similar among the 3 cell lines.
CD16: NK92γδTCR-576 and NK92γδTCR-5 are negative. A small frequency of NK92 cells were CD16 negative, indicating that NK92 is not a monoclonal cell line. Surprisingly, we observed that all γδTCR positive NK92 clones that did not respond to Daudi stimulation were CD16 positive, suggesting that CD16 inhibits γδTCR signaling by a completely unforeseen mechanism. Also surprising was the observation that CD16 positive primary γδ T cells do not show respond to IPP stimulation.
NK coreceptors: NK92γδTCR-S76 and NK92γδTCR-5 showed lower expression of 2B4 and NTB-A than NK92 cell.
CD59, DNAM-1 and NKp80: Expression was similar among the 3 cell lines.
NK inhibitory receptors: NK92γδTCR-576 and NK92γδTCR-5 expressed lower levels of NKG2A and TIM3 compared to the original NK92 cell.
PD1 and TIGIT: All 3 cell lines lacked expression of PD1 and TIGIT.

Thus, the most potent NK92γδTCR-576 had the highest levels expression of activating receptor NKp44 and the lowest expression of inhibitory receptor NKG2A. This profile is consistent with effector potency and reflects the control of tumor killing by both γδTCR and NK receptors.

### Example 25: Testing NKp44 on NK92γδTCR-S76 Function

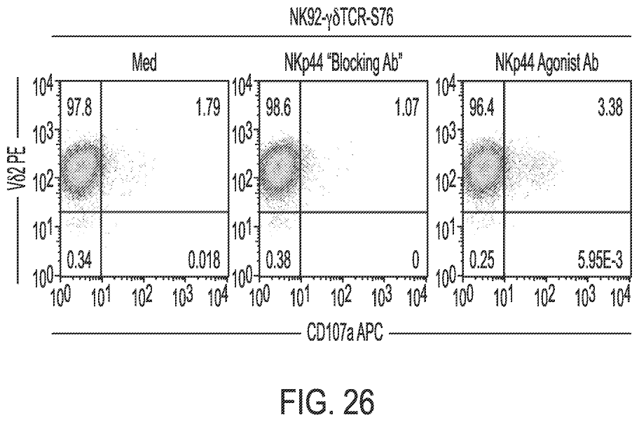

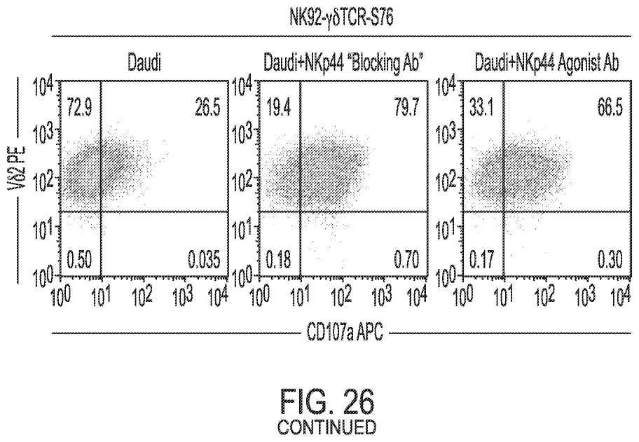

The phenotype assay showed that NK92γδTCR-576 expressed much higher levels of NKp44. We next tested whether NKp44 is responsible for higher effector function of NK92-γδTCR-S7NK92γδTCR-576 was pretreated with blocking or agonist anti-NKp44 antibodies for 1 hour. After treatment, S76 was treated with Daudi cells at 1:1 ratio for 4 hours. CD107a was measured by flow cytometry. For detecting CD107a, cells were washed, resuspended in 50-100 μl of staining buffer, and stained with mouse anti-human Vδ2-PE clone B6 and mouse anti-human CD107a-APC clone H4A3. Cells were washed with staining buffer and resuspended. Data for at least 1×10⁴ lymphocytes (gated on the basis of forward- and side-scatter profiles) were acquired from each sample on a FACSCalibur flow cytometer (BD Biosciences). All samples were analyzed using FlowJo software. As shown in FIG. 26, NKp44 antibodies significantly enhanced Daudi-induced NK92-gdTCR-S76 response. Both antibodies did not induce NK92-gdTCR-S76 response by themselves. The results showed that NKp44 can enhance NK92-gdTCR-S76 response, acting as a costimulatory receptor.

### Example 26: Lentivirus Integration Site Analysis and Expression of CD16

NK92 cell were obtained from ATCC, described as a NK92 cell line modified by retrovirus-mediated integration of an exogenous CD16 gene (Fc receptor). Exogenous CD16 was meant to enable these cells to bind antibodies for performing antibody-dependent cell mediated cytotoxicity of tumor cells. The parental NK92 cells were not available through ATCC and could not be obtained.

As described herein, we modified these NK92-CD16 cells utilizing lentivirus vectors that were distinct from the retrovirus vector originally used to introduce CD16 into the parental NK92 cells. Our lentivirus vector integrated the CD3 genes into the cell's DNA and a second integrated genes for the gamma delta T cell receptor. Cells were screened to identify ones expressing the gdTCR and then tested for potency against tumor cell lines. As described herein, we selected three clones (S76, 5 and S68) for further study and provided data attesting to their recognition of cancer cells and the important function of the gdTCR. Anther clone, S29, was selected as a control because it was transduced but did not react to tumor cells.

Unexpectedly, clones S76, 5 and S68 failed to express CD16 while the non-functional S29 clone did express CD16. In the original cell population of NK92-CD16 cells obtained from ATCC, >99% of cells expressed CD16, so these results were surprising. To further investigate, lentivirus integration site analysis was performed by Lenti-X™ Integration Site Analysis Kit from Clontech Laboratories. Through this analysis, it was determined that cloned cells lines S76, 5 and S68 were derived from the same cell after lentivirus vector transduction. Consequently, the lentivirus integration sites for these three cloned cells lines are identical (except for detecting an additional integration site in S68). The cloned S29 cell line which expressed CD16 had two lentivirus vector integration sites which were clearly distinct from the S76, 5 and S68 cell lines. Further, S76, 5 and S68 have no detectable retrovirus integration suggested they were never transduced with the CD16 retrovirus vector, or that the transduced vector had been deleted, whereas the S29 cell line possessed the expected retrovirus integration that explained its CD16+ phenotype.

Surprisingly then, it was discovered that the expression of CD16 renders NK cells incapable of using the gdTCR, and that only CD16 retrovirus-negative NK cells can express a functional gdTCR. The inventors have discovered that NK cells which lack substantial CD16 expression show enhanced expression of the Vγ9Vδ2 T cell receptor and/or enhanced recognition and lysis of cancer cells. Without being limited to any particular theory, it is believed that CD16 expression interferes with the ability of CD3 to traffic Vγ9Vδ2 T cell receptors to the cell surface of NK cells, thereby preventing recognition of cancer cell antigens.

### Example 27: Down-Regulating CD16 Expression

Levels of CD16 in NK cells are downregulated to facilitate gd TCR expression and consequent recognition and killing of tumor cells as a therapy for liquid and solid tumors.

Bioinformatic analysis is performed on the CD16 mRNA coding sequence standard analytical tools to define optimum target sites for inhibitory RNA. A collection of 20 likely target sites is compiled for the CD16 gene. Short interfering RNA (siRNA) molecules with sequences complementary to these 20 targets (guide sequences) are synthesized and obtained from a commercial supplier.

For one experiment, the siRNA guide sequences are delivered to cells in the form of plasmid constructs encoded in an integrating viral vectors. One set of twenty constructs 27A1-27A20, constructs are created where each of the 20 siRNAs is individually encoded into one of the constructs, within the backbone of short-hairpin RNA (shRNA). A promoter/enhancer region is situated upstream of the shRNA and a transcription terminator is placed downstream of the shRNA. For another set of twenty additional constructs, 27B1-27B20, constructs are created where each of the 20 siRNAs is individually encoded into one of the constructs, within the backbone of micro-RNA (miRNA). A promoter/enhancer region is situated upstream of the miRNA and a transcription terminator is placed downstream of the miRNA. Each of the forty constructs, 27A1-27A20 and 27B-27B2, are individually delivered to NK cells, where the shRNA or miRNA is expressed, respectively, and processed by cellular enzymes to release each of the siRNA sequences. The siRNA sequences anneal to CD16 mRNA at their respective target sites and promote RNA cleavage, destabilization and degradation of the CD16 mRNA. Cell transduction assays are performed to determine the extent of reduction for CD16 mRNA, and CD16 protein levels on the cell surface. The most potent siRNAs (i.e., those which lead to the lowest level of CD16 mRNA and CD16 protein expression) are selected for further experimentation and NK cell modification.

A variety of NK cells are chosen to test. Primary NK cells, immortalized NK cells, bone marrow hematopoietic stem cell precursor cells (HSCPC), and embryonic stem cells (ES cells) are obtained and modified with the constructs encoding the most potent siRNAs chosen in the previous section. CD16 mRNA and protein levels are determined and the NK cells with the lowest levels of CD16 expression. Those with the lowest expression of CD16 are tested for their ability to recognize and lyse cancer cell lines using the assays described in the previous examples. The most potent modified NK cells are implanted in vivo in subjects with cancer, producing therapeutic effect.

### Example 28: Editing the CD16 Gene

The CD16 gene is edited in NK cells to prevent expression of the CD16 protein and thus, facilitate gdTCR expression and consequent recognition and killing of tumor cells as a therapy for liquid and solid tumors.

In a first experiment, the CD16 mRNA sequence is analyzed using bioinformatic tools to define optimum target sites for CRISPR guide RNA. In a second experiment, the CD16 mRNA sequence is analyzed using bioinformatic tools to define optimum target sites for zinc-finger nuclease recognition. In a first experiment, the CD16 mRNA sequence is analyzed using bioinformatic tools to define optimum target sites for TALEN recognition sites. A collection of 20 guide sequences is compiled for each of three experiments.

The editing constructs are delivered to NK cells and recognize their respective target sequences in the CD16 gene and introduce mutations (including deletions) to inactivate gene expression. Individual gene editing constructs are compared in cell transfection or transduction assays to determine the efficient of biallelic CD16 gene modification. Limited dilution is used to isolate and grow clonal cell lines and expression of CD16 mRNA and CD16 protein is determined for each isolated cell line as described in the previous examples. Genomic DNA is purified from the cells which have the lowest levels of CD16 expression and biallelic inactivation of the CD16 gene is confirmed.

In one experiment, NK cells with biallelic modification of the CD16 alleles are transduced with integrating vectors encoding the CD3 proteins and the gamma delta T cell receptor. Cells positive for CD3 and TCR are cloned or enriched from the starting culture, cultured to increase cell numbers, and tested for activation by exposure to tumor cells and for their capacity to lyse tumor cells through cellular cytotoxicity.

In another experiment, NK cells with biallelic modification of the CD16 alleles are transduced with non-integrating vectors encoding the CD3 proteins and the gamma delta T cell receptor. Cells positive for CD3 and TCR are cloned or enriched from the starting culture, cultured to increase cell numbers, and tested for activation by exposure to tumor cells and for their capacity to lyse tumor cells through cellular cytotoxicity. The most potent modified NK cells are implanted in vivo in subjects with cancer, producing therapeutic effect.

### Example 29: Overexpressing CD3 Proteins to Facilitate Expression and Function of the Gamma Delta TCR

CD3 proteins are over-expressed in NK cells to facilitate gdTCR expression and consequent recognition and killing of tumor cells as a therapy for liquid and solid tumors.

In a first experiment, NK cells are transduced with integrating viral vectors expressing the CD3 protein coding region under control of the CMV promoter. High level expressions of the CD3 proteins are obtained. In a second experiment, NK cells are transduced with non-integrating viral vectors expressing the CD3 protein coding region under control of the CMV promoter. High level expressions of the CD3 proteins are obtained. Quantitative analysis of CD3 expression is performed using antibody staining and flow cytometry, western blotting or any suitable technique known in the art, to confirm high level CD3 expression.

In a further experiment, NK cells with high levels of CD3 protein expression are transduced with an integrating encoding the gamma delta T cell receptor. Cells positive for CD3 and gdTCR are cloned or enriched from the starting culture, cultured to increase cell numbers, and tested for activation by exposure to tumor cells and for the capacity to lyse tumor cells through cellular cytotoxicity. In another experiment, NK cells with high levels of CD3 protein expression are transduced with a non-integrating encoding the gamma delta T cell receptor. Cells positive for CD3 and gdTCR are cloned or enriched from the starting culture, cultured to increase cell numbers, and tested for activation by exposure to tumor cells and for the capacity to lyse tumor cells through cellular cytotoxicity.

Cells positive for CD3 and gd TCR are cloned or enriched from the starting culture, cultured to increase cell numbers, and tested for activation by exposure to tumor cells and for the capacity to lyse tumor cells through cellular cytotoxicity. The most potent modified NK cells are implanted in vivo in subjects with cancer, producing therapeutic effect.

### Sequences

The following sequences are referred to herein:

| SEQ | | |
|---|---|---|
| ID | | |
| NO | Description | Sequence |
| 1 | Gamma chain constant | GATAAACAACTTGATGCAGATGTTTCCCCC |
| | region (KC170727.1) | AAGCCCACTATTTTTCTTCCTTCAATTGCT |
| | | GAAACAAAGCTCCAGAAGGCTGGAACATAC |
| | | CTTTGTCTTCTTGAGAAATTTTTCCCTGAT |
| | | GTTATTAAGATACATTGGCAAGAAAAGAAG |
| | | AGCAACACGATTCTGGGATCCCAGGAGGGG |
| | | AACACCATGAAGACTAACGACACATACATG |
| | | AAATTTAGCTGGTTAACGGTGCCAGAAAAG |
| | | TCACTGGACAAAGAACACAGATGTATCGTC |
| | | AGACATGAGAATAATAAAAACGGAGTTGAT |
| | | CAAGAAATTATCTTTCCTCCAATAAAGACA |
| | | GATGTCATCACAATGGATCCCAAAGACAAT |
| | | TGTTCAAAAGATGCAAATGATACACTACTG |
| | | CTGCAGCTCACAAACACCTCTGCATATTAC |
| | | ATGTACCTCCTCCTACTCCTCAAGAGTGTG |
| | | GTCTATTTTGCCATCATCACCTGCTGTCTG |
| | | CTTAGAAGAACGGCTTTCTGCTGCAATGGA |
| | | GAGAAATCATAA |
| 2 | Delta chain constant | AGAAGTCAGCCTCATACCAAACCATCCGTT |
| | region (X73617.1) | TTTGTCATGAAAAATGGAACAAATGTCGCT |
| | | TGTCTGGTGAAGGAATTCTACCCCAAGGAT |
| | | ATAAGAATAAATCTCGTGTCATCCAAGAAG |
| | | ATAACAGAGTTTGATCCTGCTATTGTCATC |
| | | TCTCCCAGTGGGAAGTACAATGCTGTCAAG |
| | | CTTGGTAAATATGAAGATTCAAATTCAGTG |
| | | ACATGTTCAGTTCAACACGACAATAAAACT |
| | | GTGCACTCCACTGACTTTGAAGTGAAGACA |
| | | GATTCTACAGATCACGTAAAACCAAAGGAA |
| | | ACTGAAAACACAAAGCAACCTTCAAAGAGC |
| | | TGCCATAAACCCAAAGCCATAGTTCATACC |
| | | GAGAAGGTGAACATGATGTCCCTCACAGTG |
| | | CTTGGGCTACGAATGCTGTTTGCAAAGACT |
| | | GTTGCCGTCAATTTTCTCTTGACTGCCAAG |
| | | TTATTTTTCTTGTAA |
| 3 | Vγ9 variable region | ATGCTGTCACTGCTGCTCCACGCATCAACG |
| | (G115) | CTGGCAGTCCTTGGGGCTCTGTGTGTATAT |
| | | GGTGCAGGTCACCTAGAGCAACCTCAAATT |
| | | TCCAGTACTAAAACGCTGTCAAAAACAGCC |
| | | CGCCTGGAATGTGTGGTGTCTGGAATAACA |
| | | ATTTCTGCAACATCTGTATATTGGTATCGA |
| | | GAGAGACCTGGTGAAGTCATACAGTTCCTG |
| | | GTGTCCATTTCATATGACGGCACTGTCAGA |
| | | AAGGAATCCGGCATTCCGTCAGGCAAATTT |
| | | GAGGTGGATAGGATACCTGAAACGTCTACA |
| | | TCCACTCTCACCATTCACAATGTAGAGAAA |
| | | CAGGACATAGCTACCTACTACTGTGCCTTG |
| | | TGGGAGGCTCAACAGGAGTTGGGCAAAAAA |
| | | ATCAAGGTATTTGGTCCCGGAACAAAGCTT |
| | | ATCATTACA |
| 4 | Vδ2 variable region (C15) | ATGCAGAGGATCTCCTCCCTCATCCATCTC |
| | | TCTCTCTTCTGGGCAGGAGTCATGTCAGCC |
| | | ATTGAGTTGGTGCCTGAACACCAAACAGTG |
| | | CCTGTGTCAATAGGGGTCCCTGCCACCCTC |
| | | AGGTGCTCCATGAAAGGAGAAGCGATCGGT |
| | | AACTACTATATCAACTGGTACAGGAAGACC |
| | | CAAGGTAACACAATCACTTTCATATACCGA |
| | | GAAAAGGACATCTATGGCCCTGGTTTCAAA |
| | | GACAATTTCCAAGGTGACATTGATATTGCA |
| | | AAGAACCTGGCTGTACTTAAGATACTTGCA |
| | | CCATCAGAGAGAGATGAAGGGTCTTACTAC |
| | | TGTGCCTGTGACGCTCTGAAGCGCACTGAT |
| | | ACCGATAAACTCATCTTTGGAAAAGGAACC |
| | | CGTGTGACTGTGGAACCA |
| 5 | Vγ9V82 T cell receptor | ATGCTGTCACTGCTGCTCCACGCATCAACG |
| | (Vγ9-IRES-V82) | CTGGCAGTCCTTGGGGCTCTGTGTGTATAT |
| | (AGT418) | GGTGCAGGTCACCTAGAGCAACCTCAAATT |
| | | TCCAGTACTAAAACGCTGTCAAAAACAGCC |
| | | CGCCTGGAATGTGTGGTGTCTGGAATAACA |
| | | ATTTCTGCAACATCTGTATATTGGTATCGA |
| | | GAGAGACCTGGTGAAGTCATACAGTTCCTG |
| | | GTGTCCATTTCATATGACGGCACTGTCAGA |
| | | AAGGAATCCGGCATTCCGTCAGGCAAATTT |
| | | GAGGTGGATAGGATACCTGAAACGTCTACA |
| | | TCCACTCTCACCATTCACAATGTAGAGAAA |
| | | CAGGACATAGCTACCTACTACTGTGCCTTG |
| | | TGGGAGGCTCAACAGGAGTTGGGCAAAAAA |
| | | ATCAAGGTATTTGGTCCCGGAACAAAGCTT |
| | | ATCATTACAGATAAACAACTTGATGCAGAT |
| | | GTTTCCCCCAAGCCCACTATTTTTCTTCCT |
| | | TCAATTGCTGAAACAAAGCTCCAGAAGGCT |
| | | GGAACATACCTTTGTCTTCTTGAGAAATTT |
| | | TTCCCTGATGTTATTAAGATACATTGGCAA |
| | | GAAAAGAAGAGCAACACGATTCTGGGATCC |
| | | CAGGAGGGGAACACCATGAAGACTAACGAC |
| | | ACATACATGAAATTTAGCTGGTTAACGGTG |
| | | CCAGAAAAGTCACTGGACAAAGAACACAGA |
| | | TGTATCGTCAGACATGAGAATAATAAAAAC |
| | | GGAGTTGATCAAGAAATTATCTTTCCTCCA |
| | | ATAAAGACAGATGTCATCACAATGGATCCC |
| | | AAAGACAATTGTTCAAAAGATGCAAATGAT |
| | | ACACTACTGCTGCAGCTCACAAACACCTCT |
| | | GCATATTACATGTACCTCCTCCTACTCCTC |
| | | AAGAGTGTGGTCTATTTTGCCATCATCACC |
| | | TGCTGTCTGCTTAGAAGAACGGCTTTCTGC |
| | | TGCAATGGAGAGAAATCATAAGCGGCCGCC |
| | | CCCCCCTAACGTTACTGGCCGAAGCCGCTT |
| | | GGAATAAGGCCGGTGTGCGTTTGTCTATAT |
| | | GTTATTTTCCACCATATTGCCGTCTTTTGG |
| | | CAATGTGAGGGCCCGGAAACCTGGCCCTGT |
| | | CTTCTTGACGAGCATTCCTAGGGGTCTTTC |
| | | CCCTCTCGCCAAAGGAATGCAAGGTCTGTT |
| | | GAATGTCGTGAAGGAAGCAGTTCCTCTGGA |
| | | AGCTTCTTGAAGACAAACAACGTCTGTAGC |
| | | GACCCTTTGCAGGCAGCGGAACCCCCCACC |
| | | TGGCGACAGGTGCCTCTGCGGCCAAAAGCC |
| | | ACGTGTATAAGATACACCTGCAAAGGCGGC |
| | | ACAACCCCAGTGCCACGTTGTGAGTTGGAT |
| | | AGTTGTGGAAAGAGTCAAATGGCTCTCC |
| | | TCAAGCGTATTCAACAAGGGGCTGAAGGAT |
| | | GCCCAGAAGGTACCCCATTGTATGGGATCT |
| | | GATCTGGGGCCTCGGTACACATGCTTTACA |
| | | TGTGTTTAGTCGAGGTTAAAAAAACGTCTA |
| | | GGCCCCCCGAACCACGGGGACGTGGTTTTC |
| | | CTTTGAAAAACACGATGATAATATGGCCAC |
| | | AACCGCTAGCATGCAGAGGATCTCCTCCCT |
| | | CATCCATCTCTCTCTCTTCTGGGCAGGAGT |
| | | CATGTCAGCCATTGAGTTGGTGCCTGAACA |
| | | CCAAACAGTGCCTGTGTCAATAGGGGTCCC |
| | | TGCCACCCTCAGGTGCTCCATGAAAGGAGA |
| | | AGCGATCGGTAACTACTATATCAACTGGTA |
| | | CAGGAAGACCCAAGGTAACACAATCACTTT |
| | | CATATACCGAGAAAAGGACATCTATGGCCC |
| | | TGGTTTCAAAGACAATTTCCAAGGTGACAT |
| | | TGATATTGCAAAGAACCTGGCTGTACTTAA |
| | | GATACTTGCACCATCAGAGAGAGATGAAGG |
| | | GTCTTACTACTGTGCCTGTGACGCTCTGAA |
| | | GCGCACTGATACCGATAAACTCATCTTTGG |
| | | AAAAGGAACCCGTGTGACTGTGGAACCAAG |
| | | AAGTCAGCCTCATACCAAACCATCCGTTTT |
| | | TGTCATGAAAAATGGAACAAATGTCGCTTG |
| | | TCTGGTGAAGGAATTCTACCCCAAGGATAT |
| | | AAGAATAAATCTCGTGTCATCCAAGAAGAT |
| | | AACAGAGTTTGATCCTGCTATTGTCATCTC |
| | | TCCCAGTGGGAAGTACAATGCTGTCAAGCT |
| | | TGGTAAATATGAAGATTCAAATTCAGTGAC |
| | | ATGTTCAGTTCAACACGACAATAAAACTGT |
| | | GCACTCCACTGACTTTGAAGTGAAGACAGA |
| | | TTCTACAGATCACGTAAAACCAAAGGAAAC |
| | | TGAAAACACAAAGCAACCTTCAAAGAGCTG |
| | | CCATAAACCCAAAGCCATAGTTCATACCGA |
| | | GAAGGTGAACATGATGTCCCTCACAGTGCT |
| | | TGGGCTACGAATGCTGTTTGCAAAGACTGT |
| | | TGCCGTCAATTTTCTCTTGACTGCCAAGTT |
| | | ATTTTTCTTGTAA |
| 6 | IRES sequence | CCCCCCCTAACGTTACTGGCCGAAGCCGCT |
| | | TGGAATAAGGCCGGTGTGCGTTTGTCTATA |
| | | TGTTATTTTCCACCATATTGCCGTCTTTTG |
| | | GCAATGTGAGGGCCCGGAAACCTGGCCCTG |
| | | TCTTCTTGACGAGCATTCCTAGGGGTCTTT |
| | | CCCCTCTCGCCAAAGGAATGCAAGGTCTGT |
| | | TGAATGTCGTGAAGGAAGCAGTTCCTCTGG |
| | | AAGCTTCTTGAAGACAAACAACGTCTGTAG |
| | | CGACCCTTTGCAGGCAGCGGAACCCCCCAC |
| | | CTGGCGACAGGTGCCTCTGCGGCCAAAAGC |
| | | CACGTGTATAAGATACACCTGCAAAGGCGG |
| | | CACAACCCCAGTGCCACGTTGTGAGTTGGA |
| | | TAGTTGTGGAAAGAGTCAAATGGCTCTCCT |
| | | CAAGCGTATTCAACAAGGGGCTGAAGGATG |
| | | CCCAGAAGGTACCCCATTGTATGGGATCTG |
| | | ATCTGGGGCCTCGGTACACATGCTTTACAT |
| | | GTGTTTAGTCGAGGTTAAAAAAACGTCTAG |
| | | GCCCCCCGAACCACGGGGACGTGGTTTTCC |
| | | TTTGAAAAACACGATGATAATATGGCCACA |
| | | ACC |
| 7 | EF1α promoter | CCGGTGCCTAGAGAAGGTGGCGCGGGGTAA |
| | | ACTGGGAAAGTGATGTCGTGTACTGGCTCC |
| | | GCCTTTTTCCCGAGGGTGGGGGAGAACCGT |
| | | ATATAAGTGCAGTAGTCGCCGTGAACGTTC |
| | | TTTTTCGCAACGGGTTTGCCGCCAGAACAC |
| | | AGGTAAGTGCCGTGTGTGGTTCCCGCGGGC |
| | | CTGGCCTCTTTACGGGTTATGGCCCTTGCG |
| | | TGCCTTGAATTACTTCCACGCCCCTGGCTG |
| | | CAGTACGTGATTCTTGATCCCGAGCTTCGG |
| | | GTTGGAAGTGGGTGGGAGAGTTCGAGGCCT |
| | | TGCGCTTAAGGAGCCCCTTCGCCTCGTGCT |
| | | TGAGTTGAGGCCTGGCCTGGGCGCTGGGGC |
| | | CGCCGCGTGCGAATCTGGTGGCACCTTCGC |
| | | GCCTGTCTCGCTGCTTTCGATAAGTCTCTA |
| | | GCCATTTAAAATTTTTGATGACCTGCTGCG |
| | | ACGCTTTTTTTCTGGCAAGATAGTCTTGTA |
| | | AATGCGGGCCAAGATCTGCACACTGGTATT |
| | | TCGGTTTTTGGGGCCGCGGGCGGCGACGGG |
| | | GCCCGTGCGTCCCAGCGCACATGTTCGGCG |
| | | AGGCGGGGCCTGCGAGCGCGGCCACCGAGA |
| | | ATCGGACGGGGGTAGTCTCAAGCTGGCCGG |
| | | CCTGCTCTGGTGCCTGGCCTCGCGCCGCCG |
| | | TGTATCGCCCCGCCCTGGGCGGCAAGGCTG |
| | | GCCCGGTCGGCACCAGTTGCGTGAGCGGAA |
| | | AGATGGCCGCTTCCCGGCCCTGCTGCAGGG |
| | | AGCTCAAAATGGAGGACGCGGCGCTCGGGA |
| | | GAGCGGGGGGTGAGTCACCCACACAAAGGA |
| | | AAAGGGCCTTTCCGTCCTCAGCCGTCGCTT |
| | | CATGTGACTCCACGGAGTACCGGGCGCCGT |
| | | CCAGGCACCTCGATTAGTTCTCGAGCTTTT |
| | | GGAGTACGTCGTCTTTAGGTTGGGGGGAGG |
| | | GGTTTTATGCGATGGAGTTTCCCCACACTG |
| | | AGTGGGTGGAGACTGAAGTTAGGCCAGCTT |
| | | GGCACTTGATGTAATTCTCCTTGGAATTTG |
| | | CCCTTTTTGAGTTTGGATCTTGGTTCATTC |
| | | TCAAGCCTCAGACAGTGGTTCAAAGTTTTT |
| | | TTCTTCCATTTCAGGTGTCGTGA |
| 8 | CMV promoter | ACTAGTATTATGCCCAGTACATGACCTTAT |
| | | GGGACTTTCCTACTTGGCAGTACATCTACG |
| | | TATTAGTCATCGCTATTACCATGGTGATGC |
| | | GGTTTTGGCAGTACATCAATGGGCGTGGAT |
| | | AGCGGTTTGACTCACGGGGATTTCCAAGTC |
| | | TCCACCCCATTGACGTCAATGGGAGTTTGT |
| | | TTTGGCACCAAAATCAACGGGACTTTCCAA |
| | | AATGTCGTAACAACTCCGCCCCATTGACGC |
| | | AAATGGGCGGTAGGCGTGTACGGTGGGAGG |
| | | TTTATATAAGCAGAGCTCGTTTAGTGAACC |
| | | GTCAGATCGCCTGGAGACGCCATCCACGCT |
| | | GTTTTGACCTCCATAGAAGA |
| 9 | CD3 (Gamma-Delta- | ATGGAACAGGGGAAGGGCCTGGCTGTCCTC |
| | Epsilon-Zeta chain) | ATCCTGGCTATCATTCTTCTTCAAGGTACT |
| | | TTGGCCCAGTCAATCAAAGGAAACCACTTG |
| | | GTTAAGGTGTATGACTATCAAGAAGATGGT |
| | | TCGGTACTTCTGACTTGTGATGCAGAAGCC |
| | | AAAAATATCACATGGTTTAAAGATGGGAAG |
| | | ATGATCGGCTTCCTAACTGAAGATAAAAAA |
| | | AAATGGAATCTGGGAAGTAATGCCAAGGAC |
| | | CCTCGAGGGATGTATCAGTGTAAAGGATCA |
| | | CAGAACAAGTCAAAACCACTCCAAGTGTAT |
| | | TACAGAATGTGTCAGAACTGCATTGAACTA |
| | | AATGCAGCCACCATATCTGGCTTTCTCTTT |
| | | GCTGAAATCGTCAGCATTTTCGTCCTTGCT |
| | | GTTGGGGTCTACTTCATTGCTGGACAGGAT |
| | | GGAGTTCGCCAGTCGAGAGCTTCAGACAAG |
| | | CAGACTCTGTTGCCCAATGACCAGCTCTAC |
| | | CAGCCCCTCAAGGATCGAGAAGATGACCAA |
| | | TATAGTCATCTACAAGGAAACCAGTTGAGG |
| | | AGGAATGGACCGGTGAAACAGACTTTGAAT |
| | | TTTGACCTTCTCAAGTTGGCGGGAGACGTG |
| | | GAGTCCAACCCAGGGCCCATGGAACATAGC |
| | | ACGTTTCTCTCTGGCCTGGTACTGGCTACC |
| | | CTTCTCTCGCAAGTGAGCCCCTTCAAGATA |
| | | CCTATAGAGGAACTTGAGGACAGAGTGTTT |
| | | GTGAATTGCAATACCAGCATCACATGGGTA |
| | | GAGGGAACGGTGGGAACACTGCTCTCAGAC |
| | | ATTACAAGACTGGACCTGGGAAAACGCATC |
| | | CTGGACCCACGAGGAATATATAGGTGTAAT |
| | | GGGACAGATATATACAAGGACAAAGAATCT |
| | | ACCGTGCAAGTTCATTATCGAATGTGCCAG |
| | | AGCTGTGTGGAGCTGGATCCAGCCACCGTG |
| | | GCTGGCATCATTGTCACTGATGTCATTGCC |
| | | ACTCTGCTCCTTGCTTTGGGAGTCTTCTGC |
| | | TTTGCTGGACATGAGACTGGAAGGCTGTCT |
| | | GGGGCTGCCGACACACAAGCTCTGTTGAGG |
| | | AATGACCAGGTCTATCAGCCCCTCCGAGAT |
| | | CGAGATGATGCTCAGTACAGCCACCTTGGA |
| | | GGAAACTGGGCTCGGAACAAGAGATCTGAG |
| | | GGCAGAGGAAGTCTGCTAACATGCGGTGAC |
| | | GTCGAGGAGAATCCTGGCCCAATGCAGTCG |
| | | GGCACTCACTGGAGAGTTCTGGGCCTCTGC |
| | | CTCTTATCAGTTGGCGTTTGGGGGCAAGAT |
| | | GGTAATGAAGAAATGGGTGGTATTACACAG |
| | | ACACCATATAAAGTCTCCATCTCTGGAACC |
| | | ACAGTAATATTGACATGCCCT |
| | | CAGTATCCTGGATCTGAAATACTATGGCAA |
| | | CACAACGACAAGAATATTGGAGGTGATGAG |
| | | GATGATAAAAACATAGGCAGTGATGAGGAT |
| | | CACCTGTCACTGAAGGAATTTTCAGAATTG |
| | | GAGCAAAGTGGTTATTATGTCTGCTACCCC |
| | | AGAGGAAGCAAACCAGAAGATGCGAACTTT |
| | | TATCTCTACCTGAGGGCAAGAGTGTGTGAG |
| | | AACTGCATGGAGATGGATGTGATGTCGGTG |
| | | GCCACAATTGTCATAGTGGACATCTGCATC |
| | | ACTGGGGGCTTGCTGCTGCTGGTTTACTAC |
| | | TGGAGCAAGAATAGAAAGGCCAAGGCCAAG |
| | | CCTGTGACACGAGGAGCGGGTGCTGGCGGC |
| | | AGGCAAAGGGGACAAAACAAGGAGAGGCCA |
| | | CCACCTGTTCCCAACCCAGACTATGAGCCC |
| | | ATCCGGAAAGGCCAGCGGGACCTGTATTCT |
| | | GGCCTGAATCAGAGACGCATCGGATCCGCC |
| | | ACGAACTTCTCTCTGTTAAAGCAAGCAGGA |
| | | GACGTGGAAGAAAACCCCGGTCCCATGAAG |
| | | TGGAAGGCGCTTTTCACCGCGGCCATCCTG |
| | | CAGGCACAGTTGCCGATTACAGAGGCACAG |
| | | AGCTTTGGCCTGCTGGATCCCAAACTCTGC |
| | | TACCTGCTGGATGGAATCCTCTTCATCTAT |
| | | GGTGTCATTCTCACTGCCTTGTTCCTGAGA |
| | | GTGAAGTTCAGCAGGAGCGCAGACGCCCCC |
| | | GCGTACCAGCAGGGCCAGAACCAGCTCTAT |
| | | AACGAGCTCAATCTAGGACGAAGAGAGGAG |
| | | TACGATGTTTTGGACAAGAGACGTGGCCGG |
| | | GACCCTGAGATGGGGGGAAAGCCGCAGAGA |
| | | AGGAAGAACCCTCAGGAAGGCCTGTACAAT |
| | | GAACTGCAGAAAGATAAGATGGCGGAGGCC |
| | | TACAGTGAGATTGGGATGAAAGGCGAGCGC |
| | | CGGAGGGGCAAGGGGCACGATGGCCTTTAC |
| | | CAGGGTCTCAGTACAGCCACCAAGGACACC |
| | | TACGACGCCCTTCACATGCAGGCCCTGCCC |
| | | CCTCGCTAA |
| 10 | Helper/Rev; CMV early | TAGTTATTAATAGTAATCAATTACGGGGTC |
| | (CAG) enhancer; | ATTAGTTCATAGCCCATATATGGAGTTCCG |
| | Enhance | CGTTACATAACTTACGGTAAATGGCCCGCC |
| | Transcription | TGGCTGACCGCCCAACGACCCCCGCCCATT |
| | | GACGTCAATAATGACGTATGTTCCCATAGT |
| | | AACGCCAATAGGGACTTTCCATTGACGTCA |
| | | ATGGGTGGACTATTTACGGTAAACTGCCCA |
| | | CTTGGCAGTACATCAAGTGTATCATATGCC |
| | | AAGTACGCCCCCTATTGACGTCAATGACGG |
| | | TAAATGGCCCGCCTGGCATTATGCCCAGTA |
| | | CATGACCTTATGGGACTTTCCTACTTGGCA |
| | | GTACATCTACGTATTAGTCATC |
| 11 | Helper/Rev; Chicken beta | GCTATTACCATGGGTCGAGGTGAGCCCCAC |
| | actin (CAG) promoter; | GTTCTGCTTCACTCTCCCCATCTCCCCCCC |
| | Transcription | CTCCCCACCCCCAATTTTGTATTTATTTAT |
| | | TTTTTAATTATTTTGTGCAGCGATGGGGGC |
| | | GGGGGGGGGGGGGGCGCGCGCCAGGCGGGG |
| | | CGGGGCGGGGCGAGGGGCGGGGCGGGGCGA |
| | | GGCGGAGAGGTGCGGCGGCAGCCAATCAGA |
| | | GCGGCGCGCTCCGAAAGTTTCCTTTTATGG |
| | | CGAGGCGGCGGCGGCGGCGGCCCTATAAAA |
| | | AGCGAAGCGCGCGGCGGGCG |
| 12 | Helper/Rev; Chicken beta | GGAGTCGCTGCGTTGCCTTCGCCCCGTGCC |
| | actin intron; Enhance | CCGCTCCGCGCCGCCTCGCGCCGCCCGCCC |
| | gene expression | CGGCTCTGACTGACCGCGTTACTCCCACAG |
| | | GTGAGCGGGCGGGACGGCCCTTCTCCTCCG |
| | | GGCTGTAATTAGCGCTTGGTTTAATGACGG |
| | | CTCGTTTCTTTTCTGTGGCTGCGTGAAAGC |
| | | CTTAAAGGGCTCCGGGAGGGCCCTTTGTGC |
| | | GGGGGGGAGCGGCTCGGGGGGTGCGTGCGT |
| | | GTGTGTGTGCGTGGGGAGCGCCGCGTGCGG |
| | | CCCGCGCTGCCCGGCGGCTGTGAGCGCTGC |
| | | GGGCGCGGCGCGGGGCTTTGTGCGCTCCGC |
| | | GTGTGCGCGAGGGGAGCGCGGCCGGGGGCG |
| | | GTGCCCCGCGGTGCGGGGGGGCTGCGAGGG |
| | | GAACAAAGGCTGCGTGCGGGGTGTGTGCGT |
| | | GGGGGGGTGAGCAGGGGGTGTGGGCGCGGC |
| | | GGTCGGGCTGTAACCCCCCCCTGCACCCCC |
| | | CTCCCCGAGTTGCTGAGCACGGCCCGGCTT |
| | | CGGGTGCGGGGCTCCGTGCGGGGCGTGGCG |
| | | CGGGGCTCGCCGTGCCGGGCGGGGGGTGGC |
| | | GGCAGGTGGGGGTGCCGGGCGGGGCGGGGC |
| | | CGCCTCGGGCCGGGGAGGGCTCGGGGGAGG |
| | | GGCGCGGCGGCCCCGGAGCGCCGGCGGCTG |
| | | TCGAGGCGCGGCGAGCCGCAGCCATTGCCT |
| | | TTTATGGTAATCGTGCGAGAGGGCGCAGGG |
| | | ACTTCCTTTGTCCCAAATCTGGCGGAGCCG |
| | | AAATCTGGGAGGCGCCGCCGCACCCCCTCT |
| | | AGCGGGCGCGGGCGAAGCGGTGCGGCGCCG |
| | | GCAGGAAGGAAATGGGCGGGGAGGGCCTTC |
| | | GTGCGTCGCCGCGCCGCCGTCCCCTTCTCC |
| | | ATCTCCAGCCTCGGGGCTGCCGCAGGGGGA |
| | | CGGCTGCCTTCGGGGGGGACGGGGCAGGGC |
| | | GGGGTTCGGCTTCTGGCGTGTGACCGGCGG |
| 13 | Helper/Rev; HIV Gag; | ATGGGTGCGAGAGCGTCAGTATTAAGCGGG |
| | Viral capsid | GGAGAATTAGATCGATGGGAAAAAATTCGG |
| | | TTAAGGCCAGGGGGAAAGAAAAAATATAAA |
| | | TTAAAACATATAGTATGGGCAAGCAGGGAG |
| | | CTAGAACGATTCGCAGTTAATCCTGGCCTG |
| | | TTAGAAACATCAGAAGGCTGTAGACAAATA |
| | | CTGGGACAGCTACAACCATCCCTTCAGACA |
| | | GGATCAGAAGAACTTAGATCATTATATAAT |
| | | ACAGTAGCAACCCTCTATTGTGTGCATCAA |
| | | AGGATAGAGATAAAAGACACCAAGGAAGCT |
| | | TTAGACAAGATAGAGGAAGAGCAAAACAAA |
| | | AGTAAGAAAAAAGCACAGCAAGCAGCAGCT |
| | | GACACAGGACACAGCAATCAGGTCAGCCAA |
| | | AATTACCCTATAGTGCAGAACATCCAGGGG |
| | | CAAATGGTACATCAGGCCATATCACCTAGA |
| | | ACTTTAAATGCATGGGTAAAAGTAGTAGAA |
| | | GAGAAGGCTTTCAGCCCAGAAGTGATACCC |
| | | ATGTTTTCAGCATTATCAGAAGGAGCCACC |
| | | CCACAAGATTTAAACACCATGCTAAACACA |
| | | GTGGGGGGACATCAAGCAGCCATGCAAATG |
| | | TTAAAAGAGACCATCAATGAGGAAGCTGCA |
| | | GAATGGGATAGAGTGCATCCAGTGCATGCA |
| | | GGGCCTATTGCACCAGGCCAGATGAGAGAA |
| | | CCAAGGGGAAGTGACATAGCAGGAACTACT |
| | | AGTACCCTTCAGGAACAAATAGGATGGATG |
| | | ACACATAATCCACCTATCCCAGTAGGAGAA |
| | | ATCTATAAAAGATGGATAATCCTGGGATTA |
| | | AATAAAATAGTAAGAATGTATAGCCCTACC |
| | | AGCATTCTGGACATAAGACAAGGACCAAAG |
| | | GAACCCTTTAGAGACTATGTAGACCGATTC |
| | | TATAAAACTCTAAGAGCCGAGCAAGCTTCA |
| | | CAAGAGGTAAAAAATTGGATGACAGAAACC |
| | | TTGTTGGTCCAAAATGCGAACCCAGATTGT |
| | | AAGACTATTTTAAAAGCATTGGGACCAGGA |
| | | GCGACACTAGAAGAAATGATGACAGCATGT |
| | | CAGGGAGTGGGGGGACCCGGCCATAAAGCA |
| | | AGAGTTTTGGCTGAAGCAATGAGCCAAGTA |
| | | ACAAATCCAGCTACCATAATGATACAGAAA |
| | | GGCAATTTTAGGAACCAAAGAAAGACTGTT |
| | | AAGTGTTTCAATTGTGGCAAAGAAGGGCAC |
| | | ATAGCCAAAAATTGCAGGGCCCCTAGGAAA |
| | | AAGGGCTGTTGGAAATGTGGAAAGGAAGGA |
| | | CACCAAATGAAAGATTGTACTGAGAGACAG |
| | | GCTAATTTTTTAGGGAAGATCTGGCCTTCC |
| | | CACAAGGGAAGGCCAGGGAATTTTCTTCAG |
| | | AGCAGACCAGAGCCAACAGCCCCACCAGAA |
| | | GAGAGCTTCAGGTTTGGGGAAGAGACAACA |
| | | ACTCCCTCTCAGAAGCAGGAGCCGATAGAC |
| | | AAGGAACTGTATCCTTTAGCTTCCCTCAGA |
| | | TCACTCTTTGGCAGCGACCCCTCGTCACAA |
| | | TAA |
| 14 | Helper/Rev; HIV Pol; | ATGAATTTGCCAGGAAGATGGAAACCAAAA |
| | Protease and reverse | ATGATAGGGGGAATTGGAGGTTTTATCAAA |
| | transcriptase | GTAGGACAGTATGATCAGATACTCATAGAA |
| | | ATCTGCGGACATAAAGCTATAGGTACAGTA |
| | | TTAGTAGGACCTACACCTGTCAACATAATT |
| | | GGAAGAAATCTGTTGACTCAGATTGGCTGC |
| | | ACTTTAAATTTTCCCATTAGTCCTATTGAG |
| | | ACTGTACCAGTAAAATTAAAGCCAGGAATG |
| | | GATGGCCCAAAAGTTAAACAATGGCCATTG |
| | | ACAGAAGAAAAAATAAAAGCATTAGTAGAA |
| | | ATTTGTACAGAAATGGAAAAGGAAGGAAAA |
| | | ATTTCAAAAATTGGGCCTGAAAATCCATAC |
| | | AATACTCCAGTATTTGCCATAAAGAAAAAA |
| | | GACAGTACTAAATGGAGAAAATTAGTAGAT |
| | | TTCAGAGAACTTAATAAGAGAACTCAAGAT |
| | | TTCTGGGAAGTTCAATTAGGAATACCACAT |
| | | CCTGCAGGGTTAAAACAGAAAAAATCAGTA |
| | | ACAGTACTGGATGTGGGCGATGCATATTTT |
| | | TCAGTTCCCTTAGATAAAGACTTCAGGAAG |
| | | TATACTGCATTTACCATACCTAGTATAAAC |
| | | AATGAGACACCAGGGATTAGATATCAGTAC |
| | | AATGTGCTTCCACAGGGATGGAAAGGATCA |
| | | CCAGCAATATTCCAGTGTAGCATGACAAAA |
| | | ATCTTAGAGCCTTTTAGAAAACAAAATCCA |
| | | GACATAGTCATCTATCAATACATGGATGAT |
| | | TTGTATGTAGGATCTGACTTAGAAATAGGG |
| | | CAGCATAGAACAAAAATAGAGGAACTGAGA |
| | | CAACATCTGTTGAGGTGGGGATTTACCACA |
| | | CCAGACAAAAAACATCAGAAAGAACCTCCA |
| | | TTCCTTTGGATGGGTTATGAACTCCATCCT |
| | | GATAAATGGACAGTACAGCCTATAGTGCTG |
| | | CCAGAAAAGGACAGCTGGACTGTCAATGAC |
| | | ATACAGAAATTAGTGGGAAAATTGAATTGG |
| | | GCAAGTCAGATTTATGCAGGGATTAAAGTA |
| | | AGGCAATTATGTAAACTTCTTAGGGGAACC |
| | | AAAGCACTAACAGAAGTAGTACCACTAACA |
| | | GAAGAAGCAGAGCTAGAACTGGCAGAAAAC |
| | | AGGGAGATTCTAAAAGAACCGGTACATGGA |
| | | GTGTATTATGACCCATCAAAAGACTTAATA |
| | | GCAGAAATACAGAAGCAGGGGCAAGGCCAA |
| | | TGGACATATCAAATTTATCAAGAGCCATTT |
| | | AAAAATCTGAAAACAGGAAAATATGCAAGA |
| | | ATGAAGGGTGCCCACACTAATGATGTGAAA |
| | | CAATTAACAGAGGCAGTACAAAAAATAGCC |
| | | ACAGAAAGCATAGTAATATGGGGAAAGACT |
| | | CCTAAATTTAAATTACCCATACAAAAGGAA |
| | | ACATGGGAAGCATGGTGGACAGAGTATTGG |
| | | CAAGCCACCTGGATTCCTGAGTGGGAGTTT |
| | | GTCAATACCCCTCCCTTAGTGAAGTTATGG |
| | | TACCAGTTAGAGAAAGAACCCATAATAGGA |
| | | GCAGAAACTTTCTATGTAGATGGGGCAGCC |
| | | AATAGGGAAACTAAATTAGGAAAAGCAGGA |
| | | TATGTAACTGACAGAGGAAGACAAAAAGTT |
| | | GTCCCCCTAACGGACACAACAAATCAGAAG |
| | | ACTGAGTTACAAGCAATTCATCTAGCTTTG |
| | | CAGGATTCGGGATTAGAAGTAAACATAGTG |
| | | ACAGACTCACAATATGCATTGGGAATCATT |
| | | CAAGCACAACCAGATAAGAGTGAATCAGAG |
| | | TTAGTCAGTCAAATAATAGAGCAGTTAATA |
| | | AAAAAGGAAAAAGTCTACCTGGCATGGGTA |
| | | CCAGCACACAAAGGAATTGGAGGAAATGAA |
| | | CAAGTAGATGGGTTGGTCAGTGCTGGAATC |
| | | AGGAAAGTACTA |
| 15 | Helper Rev; HIV | TTTTTAGATGGAATAGATAAGGCCCAAGAA |
| | Integrase; | GAACATGAGAAATATCACAGTAATTGGAGA |
| | Integration of | GCAATGGCTAGTGATTTTAACCTACCACCT |
| | viral RNA | GTAGTAGCAAAAGAAATAGTAGCCAGCTGT |
| | | GATAAATGTCAGCTAAAAGGGGAAGCCATG |
| | | CATGGACAAGTAGACTGTAGCCCAGGAATA |
| | | TGGCAGCTAGATTGTACACATTTAGAAGGA |
| | | AAAGTTATCTTGGTAGCAGTTCATGTAGCC |
| | | AGTGGATATATAGAAGCAGAAGTAATTCCA |
| | | GCAGAGACAGGGCAAGAAACAGCATACTTC |
| | | CTCTTAAAATTAGCAGGAAGATGGCCAGTA |
| | | AAAACAGTACATACAGACAATGGCAGCAAT |
| | | TTCACCAGTACTACAGTTAAGGCCGCCTGT |
| | | TGGTGGGCGGGGATCAAGCAGGAATTTGGC |
| | | ATTCCCTACAATCCCCAAAGTCAAGGAGTA |
| | | ATAGAATCTATGAATAAAGAATTAAAGAAA |
| | | ATTATAGGACAGGTAAGAGATCAGGCTGAA |
| | | CATCTTAAGACAGCAGTACAAATGGCAGTA |
| | | TTCATCCACAATTTTAAAAGAAAAGGGGGG |
| | | ATTGGGGGGTACAGTGCAGGGGAAAGAATA |
| | | GTAGACATAATAGCAACAGACATACAAACT |
| | | AAAGAATTACAAAAACAAATTACAAAAATT |
| | | CAAAATTTTCGGGTTTATTACAGGGACAGC |
| | | AGAGATCCAGTTTGGAAAGGACCAGCAAAG |
| | | CTCCTCTGGAAAGGTGAAGGGGCAGTAGTA |
| | | ATACAAGATAATAGTGACATAAAAGTAGTG |
| | | CCAAGAAGAAAAGCAAAGATCATCAGGGAT |
| | | TATGGAAAACAGATGGCAGGTGATGATTGT |
| | | GTGGCAAGTAGACAGGATGAGGATTAA |
| 16 | Helper/Rev; HIV RRE; | AGGAGCTTTGTTCCTTGGGTTCTTGGGAGC |
| | Binds Rev element | AGCAGGAAGCACTATGGGCGCAGCGTCAAT |
| | | GACGCTGACGGTACAGGCCAGACAATTATT |
| | | GTCTGGTATAGTGCAGCAGCAGAACAATTT |
| | | GCTGAGGGCTATTGAGGCGCAACAGCATCT |
| | | GTTGCAACTCACAGTCTGGGGCATCAAGCA |
| | | GCTCCAGGCAAGAATCCTGGCTGTGGAAAG |
| | | ATACCTAAAGGATCAACAGCTCCT |
| 17 | Helper/Rev; HIV Rev; | ATGGCAGGAAGAAGCGGAGACAGCGACGAA |
| | Nuclear export and | GAACTCCTCAAGGCAGTCAGACTCATCAAG |
| | stabilize viral mRNA | TTTCTCTATCAAAGCAACCCACCTCCCAAT |
| | | CCCGAGGGGACCCGACAGGCCCGAAGGAAT |
| | | AGAAGAAGAAGGTGGAGAGAGAGACAGAGA |
| | | CAGATCCATTCGATTAGTGAACGGATCCTT |
| | | AGCACTTATCTGGGACGATCTGCGGAGCCT |
| | | GTGCCTCTTCAGCTACCACCGCTTGAGAGA |
| | | CTTACTCTTGATTGTAACGAGGATTGTGGA |
| | | ACTTCTGGGACGCAGGGGGTGGGAAGCCCT |
| | | CAAATATTGGTGGAATCTCCTACAATATTG |
| | | GAGTCAGGAGCTAAAGAATAG |
| 18 | Helper/Rev; Rabbit beta | AGATCTTTTTCCCTCTGCCAAAAATTATGG |
| | globin poly A; RNA | GGACATCATGAAGCCCCTTGAGCATCTGAC |
| | stability | TTCTGGCTAATAAAGGAAATTTATTTTCAT |
| | | TGCAATAGTGTGTTGGAATTTTTTGTGTCT |
| | | CTCACTCGGAAGGACATATGGGAGGGCAAA |
| | | TCATTTAAAACATCAGAATGAGTATTTGGT |
| | | TTAGAGTTTGGCAACATATGCCATATGCTG |
| | | GCTGCCATGAACAAAGGTGGCTATAAAGAG |
| | | GTCATCAGTATATGAAACAGCCCCCTGCTG |
| | | TCCATTCCTTATTCCATAGAAAAGCCTTGA |
| | | CTTGAGGTTAGATTTTTTTTATATTTTGTT |
| | | TTGTGTTATTTTTTTCTTTAACATCCCTAA |
| | | AATTTTCCTTACATGTTTTACTAGCCAGAT |
| | | TTTTCCTCCTCTCCTGACTACTCCCAGTCA |
| | | TAGCTGTCCCTCTTCTCTTATGAAGATC |
| 19 | Envelope; CMV | ACATTGATTATTGACTAGTTATTAATAGTA |
| | promoter; Transcription | ATCAATTACGGGGTCATTAGTTCATAGCCC |
| | | ATATATGGAGTTCCGCGTTACATAACTTAC |
| | | GGTAAATGGCCCGCCTGGCTGACCGCCCAA |
| | | CGACCCCCGCCCATTGACGTCAATAATGAC |
| | | GTATGTTCCCATAGTAACGCCAATAGGGAC |
| | | TTTCCATTGACGTCAATGGGTGGAGTATTT |
| | | ACGGTAAACTGCCCACTTGGCAGTACATCA |
| | | AGTGTATCATATGCCAAGTACGCCCCCTAT |
| | | TGACGTCAATGACGGTAAATGGCCCGCCTG |
| | | GCATTATGCCCAGTACATGACCTTATGGGA |
| | | CTTTCCTACTTGGCAGTACATCTACGTATT |
| | | AGTCATCGCTATTACCATGGTGATGCGGTT |
| | | TTGGCAGTACATCAATGGGCGTGGATAGCG |
| | | GTTTGACTCACGGGGATTTCCAAGTCTCCA |
| | | CCCCATTGACGTCAATGGGAGTTTGTTTTG |
| | | GCACCAAAATCAACGGGACTTTCCAAAATG |
| | | TCGTAACAACTCCGCCCCATTGACGCAAAT |
| | | GGGCGGTAGGCGTGTACGGTGGGAGGTCTA |
| | | TATAAGC |
| 20 | Envelope; Beta globin | GTGAGTTTGGGGACCCTTGATTGTTCTTTC |
| | intron; Enhance gene | TTTTTCGCTATTGTAAAATTCATGTTATAT |
| | expression | GGAGGGGGCAAAGTTTTCAGGGTGTTGTTT |
| | | AGAATGGGAAGATGTCCCTTGTATCACCAT |
| | | GGACCCTCATGATAATTTTGTTTCTTTCAC |
| | | TTTCTACTCTGTTGACAACCATTGTCTCCT |
| | | CTTATTTTCTTTTCATTTTCTGTAACTTTT |
| | | TCGTTAAACTTTAGCTTGCATTTGTAACGA |
| | | ATTTTTAAATTCACTTTTGTTTATTTGTCA |
| | | GATTGTAAGTACTTTCTCTAATCACTTTTT |
| | | TTTCAAGGCAATCAGGGTATATTATATTGT |
| | | ACTTCAGCACAGTTTTAGAGAACAATTGTT |
| | | ATAATTAAATGATAAGGTAGAATATTTCTG |
| | | CATATAAATTCTGGCTGGCGTGGAAATATT |
| | | CTTATTGGTAGAAACAACTACACCCTGGTC |
| | | ATCATCCTGCCTTTCTCTTTATGGTTACAA |
| | | TGATATACACTGTTTGAGATGAGGATAAAA |
| | | TACTCTGAGTCCAAACCGGGCCCCTCTGCT |
| | | AACCATGTTCATGCCTTCTTCTCTTTCCTA |
| | | CAG |
| 21 | Envelope; Rabbit beta | AGATCTTTTTCCCTCTGCCAAAAATTATGG |
| | globin poly A; RNA | GGACATCATGAAGCCCCTTGAGCATCTGAC |
| | stability | TTCTGGCTAATAAAGGAAATTTATTTTCAT |
| | | TGCAATAGTGTGTTGGAATTTTTTGTGTCT |
| | | CTCACTCGGAAGGACATATGGGAGGGCAAA |
| | | TCATTTAAAACATCAGAATGAGTATTTGGT |
| | | TTAGAGTTTGGCAACATATGCCCATATGCT |
| | | GGCTGCCATGAACAAAGGTTGGCTATAAAG |
| | | AGGTCATCAGTATATGAAACAGCCCCCTGC |
| | | TGTCCATTCCTTATTCCATAGAAAAGCCTT |
| | | GACTTGAGGTTAGATTTTTTTTATATTTTG |
| | | TTTTGTGTTATTTTTTTCTTTAACATCCCT |
| | | AAAATTTTCCTTACATGTTTTACTAGCCAG |
| | | ATTTTTCCTCCTCTCCTGACTACTCCCAGT |
| | | CATAGCTGTCCCTCTTCTCTTATGGAGATC |
| 22 | Primer | TAAGCAGAATTCATGAATTTGCCAGGAAGA |
| | | T |
| 23 | Primer | CCATACAATGAATGGACACTAGGCGGCCGC |
| | | ACGAAT |
| 24 | Gag, Pol, Integrase | GAATTCATGAATTTGCCAGGAAGATGGAAA |
| | fragment | CCAAAAATGATAGGGGGAATTGGAGGTTTT |
| | | ATCAAAGTAAGACAGTATGATCAGATACTC |
| | | ATAGAAATCTGCGGACATAAAGCTATAGGT |
| | | ACAGTATTAGTAGGACCTACACCTGTCAAC |
| | | ATAATTGGAAGAAATCTGTTGACTCAGATT |
| | | GGCTGCACTTTAAATTTTCCCATTAGTCCT |
| | | ATTGAGACTGTACCAGTAAAATTAAAGCCA |
| | | GGAATGGATGGCCCAAAAGTTAAACAATGG |
| | | CCATTGACAGAAGAAAAAATAAAAGCATTA |
| | | GTAGAAATTTGTACAGAAATGGAAAAGGAA |
| | | GGAAAAATTTCAAAAATTGGGCCTGAAAAT |
| | | CCATACAATACTCCAGTATTTGCCATAAAG |
| | | AAAAAAGACAGTACTAAATGGAGAAAATTA |
| | | GTAGATTTCAGAGAACTTAATAAGAGAACT |
| | | CAAGATTTCTGGGAAGTTCAATTAGGAATA |
| | | CCACATCCTGCAGGGTTAAAACAGAAAAAA |
| | | TCAGTAACAGTACTGGATGTGGGCGATGCA |
| | | TATTTTTCAGTTCCCTTAGATAAAGACTTC |
| | | AGGAAGTATACTGCATTTACCATACCTAGT |
| | | ATAAACAATGAGACACCAGGGATTAGATAT |
| | | CAGTACAATGTGCTTCCACAGGGATGGAAA |
| | | GGATCACCAGCAATATTCCAGTGTAGCATG |
| | | ACAAAAATCTTAGAGCCTTTTAGAAAACAA |
| | | AATCCAGACATAGTCATCTATCAATACATG |
| | | GATGATTTGTATGTAGGATCTGACTTAGAA |
| | | ATAGGGCAGCATAGAACAAAAATAGAGGAA |
| | | CTGAGACAACATCTGTTGAGGTGGGGATTT |
| | | ACCACACCAGACAAAAAACATCAGAAAGAA |
| | | CCTCCATTCCTTTGGATGGGTTATGAACTC |
| | | CATCCTGATAAATGGACAGTACAGCCTATA |
| | | GTGCTGCCAGAAAAGGACAGCTGGACTGTC |
| | | AATGACATACAGAAATTAGTGGGAAAATTG |
| | | AATTGGGCAAGTCAGATTTATGCAGGGATT |
| | | AAAGTAAGGCAATTATGTAAACTTCTTAGG |
| | | GGAACCAAAGCACTAACAGAAGTAGTACCA |
| | | CTAACAGAAGAAGCAGAGCTAGAACTGGCA |
| | | GAAAACAGGGAGATTCTAAAAGAACCGGTA |
| | | CATGGAGTGTATTATGACCCATCAAAAGAC |
| | | TTAATAGCAGAAATACAGAAGCAGGGGCAA |
| | | GGCCAATGGACATATCAAATTTATCAAGAG |
| | | CCATTTAAAAATCTGAAAACAGGAAAGTAT |
| | | GCAAGAATGAAGGGTGCCCACACTAATGAT |
| | | GTGAAACAATTAACAGAGGCAGTACAAAAA |
| | | ATAGCCACAGAAAGCATAGTAATATGGGGA |
| | | AAGACTCCTAAATTTAAATTACCCATACAA |
| | | AAGGAAACATGGGAAGCATGGTGGACAGAG |
| | | TATTGGCAAGCCACCTGGATTCCTGAGTGG |
| | | GAGTTTGTCAATACCCCTCCCTTAGTGAAG |
| | | TTATGGTACCAGTTAGAGAAAGAACCCATA |
| | | ATAGGAGCAGAAACTTTCTATGTAGATGGG |
| | | GCAGCCAATAGGGAAACTAAATTAGGAAAA |
| | | GCAGGATATGTAACTGACAGAGGAAGACAA |
| | | AAAGTTGTCCCCCTAACGGACACAACAAAT |
| | | CAGAAGACTGAGTTACAAGCAATTCATCTA |
| | | GCTTTGCAGGATTCGGGATTAGAAGTAAAC |
| | | ATAGTGACAGACTCACAATATGCATTGGGA |
| | | ATCATTCAAGCACAACCAGATAAGAGTGAA |
| | | TCAGAGTTAGTCAGTCAAATAATAGAGCAG |
| | | TTAATAAAAAAGGAAAAAGTCTACCTGGCA |
| | | TGGGTACCAGCACACAAAGGAATTGGAGGA |
| | | AATGAACAAGTAGATAAATTGGTCAGTGCT |
| | | GGAATCAGGAAAGTACTATTTTTAGATGGA |
| | | ATAGATAAGGCCCAAGAAGAACATGAGAAA |
| | | TATCACAGTAATTGGAGAGCAATGGCTAGT |
| | | GATTTTAACCTACCACCTGTAGTAGCAAAA |
| | | GAAATAGTAGCCAGCTGTGATAAATGTCAG |
| | | CTAAAAGGGGAAGCCATGCATGGACAAGTA |
| | | GACTGTAGCCCAGGAATATGGCAGCTAGAT |
| | | TGTACACATTTAGAAGGAAAAGTTATCTTG |
| | | GTAGCAGTTCATGTAGCCAGTGGATATATA |
| | | GAAGCAGAAGTAATTCCAGCAGAGACAGGG |
| | | CAAGAAACAGCATACTTCCTCTTAAAATTA |
| | | GCAGGAAGATGGCCAGTAAAAACAGTACAT |
| | | ACAGACAATGGCAGCAATTTCACCAGTACT |
| | | ACAGTTAAGGCCGCCTGTTGGTGGGCGGGG |
| | | ATCAAGCAGGAATTTGGCATTCCCTACAAT |
| | | CCCCAAAGTCAAGGAGTAATAGAATCTATG |
| | | AATAAAGAATTAAAGAAAATTATAGGACAG |
| | | GTAAGAGATCAGGCTGAACATCTTAAGACA |
| | | GCAGTACAAATGGCAGTATTCATCCACAAT |
| | | TTTAAAAGAAAAGGGGGGATTGGGGGGTAC |
| | | AGTGCAGGGGAAAGAATAGTAGACATAATA |
| | | GCAACAGACATACAAACTAAAGAATTACAA |
| | | AAACAAATTACAAAAATTCAAAATTTTCGG |
| | | GTTTATTACAGGGACAGCAGAGATCCAGTT |
| | | TGGAAAGGACCAGCAAAGCTCCTCTGGAAA |
| | | GGTGAAGGGGCAGTAGTAATACAAGATAAT |
| | | AGTGACATAAAAGTAGTGCCAAGAAGAAAA |
| | | GCAAAGATCATCAGGGATTATGGAAAACAG |
| | | ATGGCAGGTGATGATTGTGTGGCAAGTAGA |
| | | CAGGATGAGGATTAA |
| 25 | DNA Fragment | TCTAGAATGGCAGGAAGAAGCGGAGACAGC |
| | containing Rev, RRE and | GACGAAGAGCTCATCAGAACAGTCAGACTC |
| | rabbit beta globin poly A | ATCAAGCTTCTCTATCAAAGCAACCCACCT |
| | | CCCAATCCCGAGGGGACCCGACAGGCCCGA |
| | | AGGAATAGAAGAAGAAGGTGGAGAGAGAGA |
| | | CAGAGACAGATCCATTCGATTAGTGAACGG |
| | | ATCCTTGGCACTTATCTGGGACGATCTGCG |
| | | GAGCCTGTGCCTCTTCAGCTACCACCGCTT |
| | | GAGAGACTTACTCTTGATTGTAACGAGGAT |
| | | TGTGGAACTTCTGGGACGCAGGGGGTGGGA |
| | | AGCCCTCAAATATTGGTGGAATCTCCTACA |
| | | ATATTGGAGTCAGGAGCTAAAGAATAGAGG |
| | | AGCTTTGTTCCTTGGGTTCTTGGGAGCAGC |
| | | AGGAAGCACTATGGGCGCAGCGTCAATGAC |
| | | GCTGACGGTACAGGCCAGACAATTATTGTC |
| | | TGGTATAGTGCAGCAGCAGAACAATTTGCT |
| | | GAGGGCTATTGAGGCGCAACAGCATCTGTT |
| | | GCAACTCACAGTCTGGGGCATCAAGCAGCT |
| | | CCAGGCAAGAATCCTGGCTGTGGAAAGATA |
| | | CCTAAAGGATCAACAGCTCCTAGATCTTTT |
| | | TCCCTCTGCCAAAAATTATGGGGACATCAT |
| | | GAAGCCCCTTGAGCATCTGACTTCTGGCTA |
| | | ATAAAGGAAATTTATTTTCATTGCAATAGT |
| | | GTGTTGGAATTTTTTGTGTCTCTCACTCGG |
| | | AAGGACATATGGGAGGGCAAATCATTTAAA |
| | | ACATCAGAATGAGTATTTGGTTTAGAGTTT |
| | | GGCAACATATGCCATATGCTGGCTGCCATG |
| | | AACAAAGGTGGCTATAAAGAGGTCATCAGT |
| | | ATATGAAACAGCCCCCTGCTGTCCATTCCT |
| | | TATTCCATAGAAAAGCCTTGACTTGAGGTT |
| | | AGATTTTTTTTATATTTTGTTTTGTGTTAT |
| | | TTTTTTCTTTAACATCCCTAAAATTTTCCT |
| | | TACATGTTTTACTAGCCAGATTTTTCCTCC |
| | | TCTCCTGACTACTCCCAGTCATAGCTGTCC |
| | | CTCTTCTCTTATGAAGATCCCTCGACCTGC |
| | | AGCCCAAGCTTGGCGTAATCATGGTCATAG |
| | | CTGTTTCCTGTGTGAAATTGTTATCCGCTC |
| | | ACAATTCCACACAACATACGAGCCGGAAGC |
| | | ATAAAGTGTAAAGCCTGGGGTGCCTAATGA |
| | | GTGAGCTAACTCACATTAATTGCGTTGCGC |
| | | TCACTGCCCGCTTTCCAGTCGGGAAACCTG |
| | | TCGTGCCAGCGGATCCGCATCTCAATTAGT |
| | | CAGCAACCATAGTCCCGCCCCTAACTCCGC |
| | | CCATCCCGCCCCTAACTCCGCCCAGTTCCG |
| | | CCCATTCTCCGCCCCATGGCTGACTAATTT |
| | | TTTTTATTTATGCAGAGGCCGAGGCCGCCT |
| | | CGGCCTCTGAGCTATTCCAGAAGTAGTGAG |
| | | GAGGCTTTTTTGGAGGCCTAGGCTTTTGCA |
| | | AAAAGCTAACTTGTTTATTGCAGCTTATAA |
| | | TGGTTACAAATAAAGCAATAGCATCACAAA |
| | | TTTCACAAATAAAGCATTTTTTTCACTGCA |
| | | TTCTAGTTGTGGTTTGTCCAAACTCATCAA |
| | | TGTATCTTATCAGCGGCCGCCCCGGG |
| 26 | DNA fragment containing | ACGCGTTAGTTATTAATAGTAATCAATTAC |
| | the CAG | GGGGTCATTAGTTCATAGCCCATATATGGA |
| | enhancer/promoter/intron | GTTCCGCGTTACATAACTTACGGTAAATGG |
| | sequence | CCCGCCTGGCTGACCGCCCAACGACCCCCG |
| | | CCCATTGACGTCAATAATGACGTATGTTCC |
| | | CATAGTAACGCCAATAGGGACTTTCCATTG |
| | | ACGTCAATGGGTGGACTATTTACGGTAAAC |
| | | TGCCCACTTGGCAGTACATCAAGTGTATCA |
| | | TATGCCAAGTACGCCCCCTATTGACGTCAA |
| | | TGACGGTAAATGGCCCGCCTGGCATTATGC |
| | | CCAGTACATGACCTTATGGGACTTTCCTAC |
| | | TTGGCAGTACATCTACGTATTAGTCATCGC |
| | | TATTACCATGGGTCGAGGTGAGCCCCACGT |
| | | TCTGCTTCACTCTCCCCATCTCCCCCCCCT |
| | | CCCCACCCCCAATTTTGTATTTATTTATTT |
| | | TTTAATTATTTTGTGCAGCGATGGGGGCGG |
| | | GGGGGGGGGGGGCGCGCGCCAGGCGGGGCG |
| | | GGGCGGGGCGAGGGGCGGGGCGGGGCGAGG |
| | | CGGAGAGGTGCGGCGGCAGCCAATCAGAGC |
| | | GGCGCGCTCCGAAAGTTTCCTTTTATGGCG |
| | | AGGCGGCGGCGGCGGCGGCCCTATAAAAAG |
| | | CGAAGCGCGCGGCGGGCGGGAGTCGCTGCG |
| | | TTGCCTTCGCCCCGTGCCCCGCTCCGCGCC |
| | | GCCTCGCGCCGCCCGCCCCGGCTCTGACTG |
| | | ACCGCGTTACTCCCACAGGTGAGCGGGCGG |
| | | GACGGCCCTTCTCCTCCGGGCTGTAATTAG |
| | | CGCTTGGTTTAATGACGGCTCGTTTCTTTT |
| | | CTGTGGCTGCGTGAAAGCCTTAAAGGGCTC |
| | | CGGGAGGGCCCTTTGTGCGGGGGGGAGCGG |
| | | CTCGGGGGGTGCGTGCGTGTGTGTGTGCGT |
| | | GGGGAGCGCCGCGTGCGGCCCGCGCTGCCC |
| | | GGCGGCTGTGAGCGCTGCGGGCGCGGCGCG |
| | | GGGCTTTGTGCGCTCCGCGTGTGCGCGAGG |
| | | GGAGCGCGGCCGGGGGCGGTGCCCCGCGGT |
| | | GCGGGGGGGCTGCGAGGGGAACAAAGGCTG |
| | | CGTGCGGGGTGTGTGCGTGGGGGGGTGAGC |
| | | AGGGGGTGTGGGCGCGGCGGTCGGGCTGTA |
| | | ACCCCCCCCTGCACCCCCCTCCCCGAGTTG |
| | | CTGAGCACGGCCCGGCTTCGGGTGCGGGGC |
| | | TCCGTGCGGGGCGTGGCGCGGGGCTCGCCG |
| | | TGCCGGGCGGGGGGTGGCGGCAGGTGGGGG |
| | | TGCCGGGCGGGGCGGGGCCGCCTCGGGCCG |
| | | GGGAGGGCTCGGGGGAGGGGCGCGGCGGCC |
| | | CCGGAGCGCCGGCGGCTGTCGAGGCGCGGC |
| | | GAGCCGCAGCCATTGCCTTTTATGGTAATC |
| | | GTGCGAGAGGGCGCAGGGACTTCCTTTGTC |
| | | CCAAATCTGGCGGAGCCGAAATCTGGGAGG |
| | | CGCCGCCGCACCCCCTCTAGCGGGCGCGGG |
| | | CGAAGCGGTGCGGCGCCGGCAGGAAGGAAA |
| | | TGGGCGGGGAGGGCCTTCGTGCGTCGCCGC |
| | | GCCGCCGTCCCCTTCTCCATCTCCAGCCTC |
| | | GGGGCTGCCGCAGGGGGACGGCTGCCTTCG |
| | | GGGGGGACGGGGCAGGGCGGGGTTCGGCTT |
| | | CTGGCGTGTGACCGGCGGGAATTC |
| 27 | DNA fragment containing | GAATTCATGAAGTGCCTTTTGTACTTAGCC |
| | VSV-G | TTTTTATTCATTGGGGTGAATTGCAAGTTC |
| | | ACCATAGTTTTTCCACACAACCAAAAAGGA |
| | | AACTGGAAAAATGTTCCTTCTAATTACCAT |
| | | TATTGCCCGTCAAGCTCAGATTTAAATTGG |
| | | CATAATGACTTAATAGGCACAGCCTTACAA |
| | | GTCAAAATGCCCAAGAGTCACAAGGCTATT |
| | | CAAGCAGACGGTTGGATGTGTCATGCTTCC |
| | | AAATGGGTCACTACTTGTGATTTCCGCTGG |
| | | TATGGACCGAAGTATATAACACATTCCATC |
| | | CGATCCTTCACTCCATCTGTAGAACAATGC |
| | | AAGGAAAGCATTGAACAAACGAAACAAGGA |
| | | ACTTGGCTGAATCCAGGCTTCCCTCCTCAA |
| | | AGTTGTGGATATGCAACTGTGACGGATGCC |
| | | GAAGCAGTGATTGTCCAGGTGACTCCTCAC |
| | | CATGTGCTGGTTGATGAATACACAGGAGAA |
| | | TGGGTTGATTCACAGTTCATCAACGGAAAA |
| | | TGCAGCAATTACATATGCCCCACTGTCCAT |
| | | AACTCTACAACCTGGCATTCTGACTATAAG |
| | | GTCAAAGGGCTATGTGATTCTAACCTCATT |
| | | TCCATGGACATCACCTTCTTCTCAGAGGAC |
| | | GGAGAGCTATCATCCCTGGGAAAGGAGGGC |
| | | ACAGGGTTCAGAAGTAACTACTTTGCTTAT |
| | | GAAACTGGAGGCAAGGCCTGCAAAATGCAA |
| | | TACTGCAAGCATTGGGGAGTCAGACTCCCA |
| | | TCAGGTGTCTGGTTCGAGATGGCTGATAAG |
| | | GATCTCTTTGCTGCAGCCAGATTCCCTGAA |
| | | TGCCCAGAAGGGTCAAGTATCTCTGCTCCA |
| | | TCTCAGACCTCAGTGGATGTAAGTCTAATT |
| | | CAGGACGTTGAGAGGATCTTGGATTATTCC |
| | | CTCTGCCAAGAAACCTGGAGCAAAATCAGA |
| | | GCGGGTCTTCCAATCTCTCCAGTGGATCTC |
| | | AGCTATCTTGCTCCTAAAAACCCAGGAACC |
| | | GGTCCTGCTTTCACCATAATCAATGGTACC |
| | | CTAAAATACTTTGAGACCAGATACATCAGA |
| | | GTCGATATTGCTGCTCCAATCCTCTCAAGA |
| | | ATGGTCGGAATGATCAGTGGAACTACCACA |
| | | GAAAGGGAACTGTGGGATGACTGGGCACCA |
| | | TATGAAGACGTGGAAATTGGACCCAATGGA |
| | | GTTCTGAGGACCAGTTCAGGATATAAGTTT |
| | | CCTTTATACATGATTGGACATGGTATGTTG |
| | | GACTCCGATCTTCATCTTAGCTCAAAGGCT |
| | | CAGGTGTTCGAACATCCTCACATTCAAGAC |
| | | GCTGCTTCGCAACTTCCTGATGATGAGAGT |
| | | TTATTTTTTGGTGATACTGGGCTATCCAAA |
| | | AATCCAATCGAGCTTGTAGAAGGTTGGTTC |
| | | AGTAGTTGGAAAAGCTCTATTGCCTCTTTT |
| | | TTCTTTATCATAGGGTTAATCATTGGACTA |
| | | TTCTTGGTTCTCCGAGTTGGTATCCATCTT |
| | | TGCATTAAATTAAAGCACACCAAGAAAAGA |
| | | CAGATTTATACAGACATAGAGATGAGAATT |
| | | C |
| 28 | Helper/Rev; RSV | CGATGTACGGGCCAGATATACGCGTATCTG |
| | promoter; Transcription | AGGGGACTAGGGTGTGTTTAGGCGAAAAGC |
| | | GGGGCTTCGGTTGTACGCGGTTAGGAGTCC |
| | | CCTCAGGATATAGTAGTTTCGCTTTTGCAT |
| | | AGGGAGGGGGAAATGTAGTCTTATGCAATA |
| | | CACTTGTAGTCTTGCAACATGGTAACGATG |
| | | AGTTAGCAACATGCCTTACAAGGAGAGAAA |
| | | AAGCACCGTGCATGCCGATTGGTGGAAGTA |
| | | AGGTGGTACGATCGTGCCTTATTAGGAAGG |
| | | CAACAGACAGGTCTGACATGGATTGGACGA |
| | | ACCACTGAATTCCGCATTGCAGAGATAATT |
| | | GTATTTAAGTGCCTAGCTCGATACAATAAA |
| | | CGCCATTTGACCATTCACCACATTGGTGTG |
| | | CACCT |
| 29 | Helper/Rev; HIV Rev; | ATGGCAGGAAGAAGCGGAGACAGCGACGAA |
| | Nuclear export and | GAACTCCTCAAGGCAGTCAGACTCATCAAG |
| | stabilize viral mRNA | TTTCTCTATCAAAGCAACCCACCTCCCAAT |
| | | CCCGAGGGGACCCGACAGGCCCGAAGGAAT |
| | | AGAAGAAGAAGGTGGAGAGAGAGACAGAGA |
| | | CAGATCCATTCGATTAGTGAACGGATCCTT |
| | | AGCACTTATCTGGGACGATCTGCGGAGCCT |
| | | GTGCCTCTTCAGCTACCACCGCTTGAGAGA |
| | | CTTACTCTTGATTGTAACGAGGATTGTGGA |
| | | ACTTCTGGGACGCAGGGGGTGGGAAGCCCT |
| | | CAAATATTGGTGGAATCTCCTACAATATTG |
| | | GAGTCAGGAGCTAAAGAATAG |
| 30 | RSV promoter and HIV | CAATTGCGATGTACGGGCCAGATATACGCG |
| | Rev | TATCTGAGGGGACTAGGGTGTGTTTAGGCG |
| | | AAAAGCGGGGCTTCGGTTGTACGCGGTTAG |
| | | GAGTCCCCTCAGGATATAGTAGTTTCGCTT |
| | | TTGCATAGGGAGGGGGAAATGTAGTCTTAT |
| | | GCAATACACTTGTAGTCTTGCAACATGGTA |
| | | ACGATGAGTTAGCAACATGCCTTACAAGGA |
| | | GAGAAAAAGCACCGTGCATGCCGATTGGTG |
| | | GAAGTAAGGTGGTACGATCGTGCCTTATTA |
| | | GGAAGGCAACAGACAGGTCTGACATGGATT |
| | | GGACGAACCACTGAATTCCGCATTGCAGAG |
| | | ATAATTGTATTTAAGTGCCTAGCTCGATAC |
| | | AATAAACGCCATTTGACCATTCACCACATT |
| | | GGTGTGCACCTCCAAGCTCGAGCTCGTTTA |
| | | GTGAACCGTCAGATCGCCTGGAGACGCCAT |
| | | CCACGCTGTTTTGACCTCCATAGAAGACAC |
| | | CGGGACCGATCCAGCCTCCCCTCGAAGCTA |
| | | GCGATTAGGCATCTCCTATGGCAGGAAGAA |
| | | GCGGAGACAGCGACGAAGAACTCCTCAAGG |
| | | CAGTCAGACTCATCAAGTTTCTCTATCAAA |
| | | GCAACCCACCTCCCAATCCCGAGGGGACCC |
| | | GACAGGCCCGAAGGAATAGAAGAAGAAGGT |
| | | GGAGAGAGAGACAGAGACAGATCCATTCGA |
| | | TTAGTGAACGGATCCTTAGCACTTATCTGG |
| | | GACGATCTGCGGAGCCTGTGCCTCTTCAGC |
| | | TACCACCGCTTGAGAGACTTACTCTTGATT |
| | | GTAACGAGGATTGTGGAACTTCTGGGACGC |
| | | AGGGGGTGGGAAGCCCTCAAATATTGGTGG |
| | | AATCTCCTACAATATTGGAGTCAGGAGCTA |
| | | AAGAATAGTCTAGA |
| 31 | Elongation Factor-1 alpha | CCGGTGCCTAGAGAAGGTGGCGCGGGGTAA |
| | (EF1-alpha) promoter | ACTGGGAAAGTGATGTCGTGTACTGGCTCC |
| | | GCCTTTTTCCCGAGGGTGGGGGAGAACCGT |
| | | ATATAAGTGCAGTAGTCGCCGTGAACGTTC |
| | | TTTTTCGCAACGGGTTTGCCGCCAGAACAC |
| | | AGGTAAGTGCCGTGTGTGGTTCCCGCGGGC |
| | | CTGGCCTCTTTACGGGTTATGGCCCTTGCG |
| | | TGCCTTGAATTACTTCCACGCCCCTGGCTG |
| | | CAGTACGTGATTCTTGATCCCGAGCTTCGG |
| | | GTTGGAAGTGGGTGGGAGAGTTCGAGGCCT |
| | | TGCGCTTAAGGAGCCCCTTCGCCTCGTGCT |
| | | TGAGTTGAGGCCTGGCCTGGGCGCTGGGGC |
| | | CGCCGCGTGCGAATCTGGTGGCACCTTCGC |
| | | GCCTGTCTCGCTGCTTTCGATAAGTCTCTA |
| | | GCCATTTAAAATTTTTGATGACCTGCTGCG |
| | | ACGCTTTTTTTCTGGCAAGATAGTCTTGTA |
| | | AATGCGGGCCAAGATCTGCACACTGGTATT |
| | | TCGGTTTTTGGGGCCGCGGGCGGCGACGGG |
| | | GCCCGTGCGTCCCAGCGCACATGTTCGGCG |
| | | AGGCGGGGCCTGCGAGCGCGGCCACCGAGA |
| | | ATCGGACGGGGGTAGTCTCAAGCTGGCCGG |
| | | CCTGCTCTGGTGCCTGGCCTCGCGCCGCCG |
| | | TGTATCGCCCCGCCCTGGGCGGCAAGGCTG |
| | | GCCCGGTCGGCACCAGTTGCGTGAGCGGAA |
| | | AGATGGCCGCTTCCCGGCCCTGCTGCAGGG |
| | | AGCTCAAAATGGAGGACGCGGCGCTCGGGA |
| | | GAGCGGGCGGGTGAGTCACCCACACAAAGG |
| | | AAAAGGGCCTTTCCGTCCTCAGCCGTCGCT |
| | | TCATGTGACTCCACGGAGTACCGGGCGCCG |
| | | TCCAGGCACCTCGATTAGTTCTCGAGCTTT |
| | | TGGAGTACGTCGTCTTTAGGTTGGGGGGAG |
| | | GGGTTTTATGCGATGGAGTTTCCCCACACT |
| | | GAGTGGGTGGAGACTGAAGTTAGGCCAGCT |
| | | TGGCACTTGATGTAATTCTCCTTGGAATTT |
| | | GCCCTTTTTGAGTTTGGATCTTGGTTCATT |
| | | CTCAAGCCTCAGACAGTGGTTCAAAGTTTT |
| | | TTTCTTCCATTTCAGGTGTCGTGA |
| 32 | Promoter; PGK | GGGGTTGGGGTTGCGCCTTTTCCAAGGCAG |
| | | CCCTGGGTTTGCGCAGGGACGCGGCTGCTC |
| | | TGGGCGTGGTTCCGGGAAACGCAGCGGCGC |
| | | CGACCCTGGGTCTCGCACATTCTTCACGTC |
| | | CGTTCGCAGCGTCACCCGGATCTTCGCCGC |
| | | TACCCTTGTGGGCCCCCCGGCGACGCTTCC |
| | | TGCTCCGCCCCTAAGTCGGGAAGGTTCCTT |
| | | GCGGTTCGCGGCGTGCCGGACGTGACAAAC |
| | | GGAAGCCGCACGTCTCACTAGTACCCTCGC |
| | | AGACGGACAGCGCCAGGGAGCAATGGCAGC |
| | | GCGCCGACCGCGATGGGCTGTGGCCAATAG |
| | | CGGCTGCTCAGCAGGGCGCGCCGAGAGCAG |
| | | CGGCCGGGAAGGGGCGGTGCGGGAGGCGGG |
| | | GTGTGGGGCGGTAGTGTGGGCCCTGTTCCT |
| | | GCCCGCGCGGTGTTCCGCATTCTGCAAGCC |
| | | TCCGGAGCGCACGTCGGCAGTCGGCTCCCT |
| | | CGTTGACCGAATCACCGACCTCTCTCCCCA |
| | | G |
| 33 | Promoter; UbC | GCGCCGGGTTTTGGCGCCTCCCGCGGGCGC |
| | | CCCCCTCCTCACGGCGAGCGCTGCCACGTC |
| | | AGACGAAGGGCGCAGGAGCGTTCCTGATCC |
| | | TTCCGCCCGGACGCTCAGGACAGCGGCCCG |
| | | CTGCTCATAAGACTCGGCCTTAGAACCCCA |
| | | GTATCAGCAGAAGGACATTTTAGGACGGGA |
| | | CTTGGGTGACTCTAGGGCACTGGTTTTCTT |
| | | TCCAGAGAGCGGAACAGGCGAGGAAAAGTA |
| | | GTCCCTTCTCGGCGATTCTGCGGAGGGATC |
| | | TCCGTGGGGCGGTGAACGCCGATGATTATA |
| | | TAAGGACGCGCCGGGTGTGGCACAGCTAGT |
| | | TCCGTCGCAGCCGGGATTTGGGTCGCGGTT |
| | | CTTGTTTGTGGATCGCTGTGATCGTCACTT |
| | | GGTGAGTTGCGGGCTGCTGGGCTGGCCGGG |
| | | GCTTTCGTGGCCGCCGGGCCGCTCGGTGGG |
| | | ACGGAAGCGTGTGGAGAGACCGCCAAGGGC |
| | | TGTAGTCTGGGTCCGCGAGCAAGGTTGCCC |
| | | TGAACTGGGGGTTGGGGGGAGCGCACAAAA |
| | | TGGCGGCTGTTCCCGAGTCTTGAATGGAAG |
| | | ACGCTTGTAAGGCGGGCTGTGAGGTCGTTG |
| | | AAACAAGGTGGGGGGCATGGTGGGCGGCAA |
| | | GAACCCAAGGTCTTGAGGCCTTCGCTAATG |
| | | CGGGAAAGCTCTTATTCGGGTGAGATGGGC |
| | | TGGGGCACCATCTGGGGACCCTGACGTGAA |
| | | GTTTGTCACTGACTGGAGAACTCGGGTTTG |
| | | TCGTCTGGTTGCGGGGGCGGCAGTTATGCG |
| | | GTGCCGTTGGGCAGTGCACCCGTACCTTTG |
| | | GGAGCGCGCGCCTCGTCGTGTCGTGACGTC |
| | | ACCCGTTCTGTTGGCTTATAATGCAGGGTG |
| | | GGGCCACCTGCCGGTAGGTGTGCGGTAGGC |
| | | TTTTCTCCGTCGCAGGACGCAGGGTTCGGG |
| | | CCTAGGGTAGGCTCTCCTGAATCGACAGGC |
| | | GCCGGACCTCTGGTGAGGGGAGGGATAAGT |
| | | GAGGCGTCAGTTTCTTTGGTCGGTTTTATG |
| | | TACCTATCTTCTTAAGTAGCTGAAGCTCCG |
| | | GTTTTGAACTATGCGCTCGGGGTTGGCGAG |
| | | TGTGTTTTGTGAAGTTTTTTAGGCACCTTT |
| | | TGAAATGTAATCATTTGGGTCAATATGTAA |
| | | TTTTCAGTGTTAGACTAGTAAA |
| 34 | Poly A; SV40 | GTTTATTGCAGCTTATAATGGTTACAAATA |
| | | AAGCAATAGCATCACAAATTTCACAAATAA |
| | | AGCATTTTTTTCACTGCATTCTAGTTGTGG |
| | | TTTGTCCAAACTCATCAATGTATCTTATCA |
| 35 | Poly A; bGH | GACTGTGCCTTCTAGTTGCCAGCCATCTGT |
| | | TGTTTGCCCCTCCCCCGTGCCTTCCTTGAC |
| | | CCTGGAAGGTGCCACTCCCACTGTCCTTTC |
| | | CTAATAAAATGAGGAAATTGCATCGCATTG |
| | | TCTGAGTAGGTGTCATTCTATTCTGGGGGG |
| | | TGGGGTGGGGCAGGACAGCAAGGGGGAGGA |
| | | TTGGGAAGACAATAGCAGGCATGCTGGGGA |
| | | TGCGGTGGGCTCTATGG |
| 36 | Envelope; RD114 | ATGAAACTCCCAACAGGAATGGTCATTTTA |
| | | TGTAGCCTAATAATAGTTCGGGCAGGGTTT |
| | | GACGACCCCCGCAAGGCTATCGCATTAGTA |
| | | CAAAAACAACATGGTAAACCATGCGAATGC |
| | | AGCGGAGGGCAGGTATCCGAGGCCCCACCG |
| | | AACTCCATCCAACAGGTAACTTGCCCAGGC |
| | | AAGACGGCCTACTTAATGACCAACCAAAAA |
| | | TGGAAATGCAGAGTCACTCCAAAAAATCTC |
| | | ACCCCTAGCGGGGGAGAACTCCAGAACTGC |
| | | CCCTGTAACACTTTCCAGGACTCGATGCAC |
| | | AGTTCTTGTTATACTGAATACCGGCAATGC |
| | | AGGGCGAATAATAAGACATACTACACGGCC |
| | | ACCTTGCTTAAAATACGGTCTGGGAGCCTC |
| | | AACGAGGTACAGATATTACAAAACCCCAAT |
| | | CAGCTCCTACAGTCCCCTTGTAGGGGCTCT |
| | | ATAAATCAGCCCGTTTGCTGGAGTGCCACA |
| | | GCCCCCATCCATATCTCCGATGGTGGAGGA |
| | | CCCCTCGATACTAAGAGAGTGTGGACAGTC |
| | | CAAAAAAGGCTAGAACAAATTCATAAGGCT |
| | | ATGCATCCTGAACTTCAATACCACCCCTTA |
| | | GCCCTGCCCAAAGTCAGAGATGACCTTAGC |
| | | CTTGATGCACGGACTTTTGATATCCTGAAT |
| | | ACCACTTTTAGGTTACTCCAGATGTCCAAT |
| | | TTTAGCCTTGCCCAAGATTGTTGGCTCTGT |
| | | TTAAAACTAGGTACCCCTACCCCTCTTGCG |
| | | ATACCCACTCCCTCTTTAACCTACTCCCTA |
| | | GCAGACTCCCTAGCGAATGCCTCCTGTCAG |
| | | ATTATACCTCCCCTCTTGGTTCAACCGATG |
| | | CAGTTCTCCAACTCGTCCTGTTTATCTTCC |
| | | CCTTTCATTAACGATACGGAACAAATAGAC |
| | | TTAGGTGCAGTCACCTTTACTAACTGCACC |
| | | TCTGTAGCCAATGTCAGTAGTCCTTTATGT |
| | | GCCCTAAACGGGTCAGTCTTCCTCTGTGGA |
| | | AATAACATGGCATACACCTATTTACCCCAA |
| | | AACTGGACAGGACTTTGCGTCCAAGCCTCC |
| | | CTCCTCCCCGACATTGACATCATCCCGGGG |
| | | GATGAGCCAGTCCCCATTCCTGCCATTGAT |
| | | CATTATATACATAGACCTAAACGAGCTGTA |
| | | CAGTTCATCCCTTTACTAGCTGGACTGGGA |
| | | ATCACCGCAGCATTCACCACCGGAGCTACA |
| | | GGCCTAGGTGTCTCCGTCACCCAGTATACA |
| | | AAATTATCCCATCAGTTAATATCTGATGTC |
| | | CAAGTCTTATCCGGTACCATACAAGATTTA |
| | | CAAGACCAGGTAGACTCGTTAGCTGAAGTA |
| | | GTTCTCCAAAATAGGAGGGGACTGGACCTA |
| | | CTAACGGCAGAACAAGGAGGAATTTGTTTA |
| | | GCCTTACAAGAAAAATGCTGTTTTTATGCT |
| | | AACAAGTCAGGAATTGTGAGAAACAAAATA |
| | | AGAACCCTACAAGAAGAATTACAAAAACGC |
| | | AGGGAAAGCCTGGCATCCAACCCTCTCTGG |
| | | ACCGGGCTGCAGGGCTTTCTTCCGTACCTC |
| | | CTACCTCTCCTGGGACCCCTACTCACCCTC |
| | | CTACTCATACTAACCATTGGGCCATGCGTT |
| | | TTCAATCGATTGGTCCAATTTGTTAAAGAC |
| | | AGGATCTCAGTGGTCCAGGCTCTGGTTTTG |
| | | ACTCAGCAATATCACCAGCTAAAACCCATA |
| | | GAGTACGAGCCATGA |
| 37 | Envelope; GALV | ATGCTTCTCACCTCAAGCCCGCACCACCTT |
| | | CGGCACCAGATGAGTCCTGGGAGCTGGAAA |
| | | AGACTGATCATCCTCTTAAGCTGCGTATTC |
| | | GGAGACGGCAAAACGAGTCTGCAGAATAAG |
| | | AACCCCCACCAGCCTGTGACCCTCACCTGG |
| | | CAGGTACTGTCCCAAACTGGGGACGTTGTC |
| | | TGGGACAAAAAGGCAGTCCAGCCCCTTTGG |
| | | ACTTGGTGGCCCTCTCTTACACCTGATGTA |
| | | TGTGCCCTGGCGGCCGGTCTTGAGTCCTGG |
| | | GATATCCCGGGATCCGATGTATCGTCCTCT |
| | | AAAAGAGTTAGACCTCCTGATTCAGACTAT |
| | | ACTGCCGCTTATAAGCAAATCACCTGGGGA |
| | | GCCATAGGGTGCAGCTACCCTCGGGCTAGG |
| | | ACCAGGATGGCAAATTCCCCCTTCTACGTG |
| | | TGTCCCCGAGCTGGCCGAACCCATTCAGAA |
| | | GCTAGGAGGTGTGGGGGGCTAGAATCCCTA |
| | | TACTGTAAAGAATGGAGTTGTGAGACCACG |
| | | GGTACCGTTTATTGGCAACCCAAGTCCTCA |
| | | TGGGACCTCATAACTGTAAAATGGGACCAA |
| | | AATGTGAAATGGGAGCAAAAATTTCAAAAG |
| | | TGTGAACAAACCGGCTGGTGTAACCCCCTC |
| | | AAGATAGACTTCACAGAAAAAGGAAAACTC |
| | | TCCAGAGATTGGATAACGGAAAAAACCTGG |
| | | GAATTAAGGTTCTATGTATATGGACACCCA |
| | | GGCATACAGTTGACTATCCGCTTAGAGGTC |
| | | ACTAACATGCCGGTTGTGGCAGTGGGCCCA |
| | | GACCCTGTCCTTGCGGAACAGGGACCTCCT |
| | | AGCAAGCCCCTCACTCTCCCTCTCTCCCCA |
| | | CGGAAAGCGCCGCCCACCCCTCTACCCCCG |
| | | GCGGCTAGTGAGCAAACCCCTGCGGTGCAT |
| | | GGAGAAACTGTTACCCTAAACTCTCCGCCT |
| | | CCCACCAGTGGCGACCGACTCTTTGGCCTT |
| | | GTGCAGGGGGCCTTCCTAACCTTGAATGCT |
| | | ACCAACCCAGGGGCCACTAAGTCTTGCTGG |
| | | CTCTGTTTGGGCATGAGCCCCCCTTATTAT |
| | | GAAGGGATAGCCTCTTCAGGAGAGGTCGCT |
| | | TATACCTCCAACCATACCCGATGCCACTGG |
| | | GGGGCCCAAGGAAAGCTTACCCTCACTGAG |
| | | GTCTCCGGACTCGGGTCATGCATAGGGAAG |
| | | GTGCCTCTTACCCATCAACATCTTTGCAAC |
| | | CAGACCTTACCCATCAATTCCTCTAAAAAC |
| | | CATCAGTATCTGCTCCCCTCAAACCATAGC |
| | | TGGTGGGCCTGCAGCACTGGCCTCACCCCC |
| | | TGCCTCTCCACCTCAGTTTTTAATCAGTCT |
| | | AAAGACTTCTGTGTCCAGGTCCAGCTGATC |
| | | CCCCGCATCTATTACCATTCTGAAGAAACC |
| | | TTGTTACAAGCCTATGACAAATCACCCCCC |
| | | AGGTTTAAAAGAGAGCCTGCCTCACTTACC |
| | | CTAGCTGTCTTCCTGGGGTTAGGGATTGCG |
| | | GCAGGTATAGGTACTGGCTCAACCGCCCTA |
| | | ATTAAAGGGCCCATAGACCTCCAGCAAGGC |
| | | CTAACCAGCCTCCAAATCGCCATTGACGCT |
| | | GACCTCCGGGCCCTTCAGGACTCAATCAGC |
| | | AAGCTAGAGGACTCACTGACTTCCCTATCT |
| | | GAGGTAGTACTCCAAAATAGGAGAGGCCTT |
| | | GACTTACTATTCCTTAAAGAAGGAGGCCTC |
| | | TGCGCGGCCCTAAAAGAAGAGTGCTGTTTT |
| | | TATGTAGACCACTCAGGTGCAGTACGAGAC |
| | | TCCATGAAAAAACTTAAAGAAAGACTAGAT |
| | | AAAAGACAGTTAGAGCGCCAGAAAAACCAA |
| | | AACTGGTATGAAGGGTGGTTCAATAACTCC |
| | | CCTTGGTTTACTACCCTACTATCAACCATC |
| | | GCTGGGCCCCTATTGCTCCTCCTTTTGTTA |
| | | CTCACTCTTGGGCCCTGCATCATCAATAAA |
| | | TTAATCCAATTCATCAATGATAGGATAAGT |
| | | GCAGTCAAAATTTTAGTCCTTAGACAGAAA |
| | | TATCAGACCCTAGATAACGAGGAAAACCTT |
| | | TAA |
| 38 | Envelope; FUG | ATGGTTCCGCAGGTTCTTTTGTTTGTACTC |
| | | CTTCTGGGTTTTTCGTTGTGTTTCGGGAAG |
| | | TTCCCCATTTACACGATACCAGACGAACTT |
| | | GGTCCCTGGAGCCCTATTGACATACACCAT |
| | | CTCAGCTGTCCAAATAACCTGGTTGTGGAG |
| | | GATGAAGGATGTACCAACCTGTCCGAGTTC |
| | | TCCTACATGGAACTCAAAGTGGGATACATC |
| | | TCAGCCATCAAAGTGAACGGGTTCACTTGC |
| | | ACAGGTGTTGTGACAGAGGCAGAGACCTAC |
| | | ACCAACTTTGTTGGTTATGTCACAACCACA |
| | | TTCAAGAGAAAGCATTTCCGCCCCACCCCA |
| | | GACGCATGTAGAGCCGCGTATAACTGGAAG |
| | | ATGGCCGGTGACCCCAGATATGAAGAGTCC |
| | | CTACACAATCCATACCCCGACTACCACTGG |
| | | CTTCGAACTGTAAGAACCACCAAAGAGTCC |
| | | CTCATTATCATATCCCCAAGTGTGACAGAT |
| | | TTGGACCCATATGACAAATCCCTTCACTCA |
| | | AGGGTCTTCCCTGGCGGAAAGTGCTCAGGA |
| | | ATAACGGTGTCCTCTACCTACTGCTCAACT |
| | | AACCATGATTACACCATTTGGATGCCCGAG |
| | | AATCCGAGACCAAGGACACCTTGTGACATT |
| | | TTTACCAATAGCAGAGGGAAGAGAGCATCC |
| | | AACGGGAACAAGACTTGCGGCTTTGTGGAT |
| | | GAAAGAGGCCTGTATAAGTCTCTAAAAGGA |
| | | GCATGCAGGCTCAAGTTATGTGGAGTTCTT |
| | | GGACTTAGACTTATGGATGGAACATGGGTC |
| | | GCGATGCAAACATCAGATGAGACCAAATGG |
| | | TGCCCTCCAGATCAGTTGGTGAATTTGCAC |
| | | GACTTTCGCTCAGACGAGATCGAGCATCTC |
| | | GTTGTGGAGGAGTTAGTTAAGAAAAGAGAG |
| | | GAATGTCTGGATGCATTAGAGTCCATCATG |
| | | ACCACCAAGTCAGTAAGTTTCAGACGTCTC |
| | | AGTCACCTGAGAAAACTTGTCCCAGGGTTT |
| | | GGAAAAGCATATACCATATTCAACAAAACC |
| | | TTGATGGAGGCTGATGCTCACTACAAGTCA |
| | | GTCCGGACCTGGAATGAGATCATCCCCTCA |
| | | AAAGGGTGTTTGAAAGTTGGAGGAAGGTGC |
| | | CATCCTCATGTGAACGGGGTGTTTTTCAAT |
| | | GGTATAATATTAGGGCCTGACGACCATGTC |
| | | CTAATCCCAGAGATGCAATCATCCCTCCTC |
| | | CAGCAACATATGGAGTTGTTGGAATCTTCA |
| | | GTTATCCCCCTGATGCACCCCCTGGCAGAC |
| | | CCTTCTACAGTTTTCAAAGAAGGTGATGAG |
| | | GCTGAGGATTTTGTTGAAGTTCACCTCCCC |
| | | GATGTGTACAAACAGATCTCAGGGGTTGAC |
| | | CTGGGTCTCCCGAACTGGGGAAAGTATGTA |
| | | TTGATGACTGCAGGGGCCATGATTGGCCTG |
| | | GTGTTGATATTTTCCCTAATGACATGGTGC |
| | | AGAGTTGGTATCCATCTTTGCATTAAATTA |
| | | AAGCACACCAAGAAAAGACAGATTTATACA |
| | | GACATAGAGATGAACCGACTTGGAAAGTAA |
| 39 | Envelope; LCMV | ATGGGTCAGATTGTGACAATGTTTGAGGCT |
| | | CTGCCTCACATCATCGATGAGGTGATCAAC |
| | | ATTGTCATTATTGTGCTTATCGTGATCACG |
| | | GGTATCAAGGCTGTCTACAATTTTGCCACC |
| | | TGTGGGATATTCGCATTGATCAGTTTCCTA |
| | | CTTCTGGCTGGCAGGTCCTGTGGCATGTAC |
| | | GGTCTTAAGGGACCCGACATTTACAAAGGA |
| | | GTTTACCAATTTAAGTCAGTGGAGTTTGAT |
| | | ATGTCACATCTGAACCTGACCATGCCCAAC |
| | | GCATGTTCAGCCAACAACTCCCACCATTAC |
| | | ATCAGTATGGGGACTTCTGGACTAGAATTG |
| | | ACCTTCACCAATGATTCCATCATCAGTCAC |
| | | AACTTTTGCAATCTGACCTCTGCCTTCAAC |
| | | AAAAAGACCTTTGACCACACACTCATGAGT |
| | | ATAGTTTCGAGCCTACACCTCAGTATCAGA |
| | | GGGAACTCCAACTATAAGGCAGTATCCTGC |
| | | GACTTCAACAATGGCATAACCATCCAATAC |
| | | AACTTGACATTCTCAGATCGACAAAGTGCT |
| | | CAGAGCCAGTGTAGAACCTTCAGAGGTAGA |
| | | GTCCTAGATATGTTTAGAACTGCCTTCGGG |
| | | GGGAAATACATGAGGAGTGGCTGGGGCTGG |
| | | ACAGGCTCAGATGGCAAGACCACCTGGTGT |
| | | AGCCAGACGAGTTACCAATACCTGATTATA |
| | | CAAAATAGAACCTGGGAAAACCACTGCACA |
| | | TATGCAGGTCCTTTTGGGATGTCCAGGATT |
| | | CTCCTTTCCCAAGAGAAGACTAAGTTCTTC |
| | | ACTAGGAGACTAGCGGGCACATTCACCTGG |
| | | ACTTTGTCAGACTCTTCAGGGGTGGAGAAT |
| | | CCAGGTGGTTATTGCCTGACCAAATGGATG |
| | | ATTCTTGCTGCAGAGCTTAAGTGTTTCGGG |
| | | AACACAGCAGTTGCGAAATGCAATGTAAAT |
| | | CATGATGCCGAATTCTGTGACATGCTGCGA |
| | | CTAATTGACTACAACAAGGCTGCTTTGAGT |
| | | AAGTTCAAAGAGGACGTAGAATCTGCCTTG |
| | | CACTTATTCAAAACAACAGTGAATTCTTTG |
| | | ATTTCAGATCAACTACTGATGAGGAACCAC |
| | | TTGAGAGATCTGATGGGGGTGCCATATTGC |
| | | AATTACTCAAAGTTTTGGTACCTAGAACAT |
| | | GCAAAGACCGGCGAAACTAGTGTCCCCAAG |
| | | TGCTGGCTTGTCACCAATGGTTCTTACTTA |
| | | AATGAGACCCACTTCAGTGATCAAATCGAA |
| | | CAGGAAGCCGATAACATGATTACAGAGATG |
| | | TTGAGGAAGGATTACATAAAGAGGCAGGGG |
| | | AGTACCCCCCTAGCATTGATGGACCTTCTG |
| | | ATGTTTTCCACATCTGCATATCTAGTCAGC |
| | | ATCTTCCTGCACCTTGTCAAAATACCAACA |
| | | CACAGGCACATAAAAGGTGGCTCATGTCCA |
| | | AAGCCACACCGATTAACCAACAAAGGAATT |
| | | TGTAGTTGTGGTGCATTTAAGGTGCCTGGT |
| | | GTAAAAACCGTCTGGAAAAGACGCTGA |
| 40 | Envelope; FPV | ATGAACACTCAAATCCTGGTTTTCGCCCTT |
| | | GTGGCAGTCATCCCCACAAATGCAGACAAA |
| | | ATTTGTCTTGGACATCATGCTGTATCAAAT |
| | | GGCACCAAAGTAAACACACTCACTGAGAGA |
| | | GGAGTAGAAGTTGTCAATGCAACGGAAACA |
| | | GTGGAGCGGACAAACATCCCCAAAATTTGC |
| | | TCAAAAGGGAAAAGAACCACTGATCTTGGC |
| | | CAATGCGGACTGTTAGGGACCATTACCGGA |
| | | CCACCTCAATGCGACCAATTTCTAGAATTT |
| | | TCAGCTGATCTAATAATCGAGAGACGAGAA |
| | | GGAAATGATGTTTGTTACCCGGGGAAGTTT |
| | | GTTAATGAAGAGGCATTGCGACAAATCCTC |
| | | AGAGGATCAGGTGGGATTGACAAAGAAACA |
| | | ATGGGATTCACATATAGTGGAATAAGGACC |
| | | AACGGAACAACTAGTGCATGTAGAAGATCA |
| | | GGGTCTTCATTCTATGCAGAAATGGAGTGG |
| | | CTCCTGTCAAATACAGACAATGCTGCTTTC |
| | | CCACAAATGACAAAATCATACAAAAACACA |
| | | AGGAGAGAATCAGCTCTGATAGTCTGGGGA |
| | | ATCCACCATTCAGGATCAACCACCGAACAG |
| | | ACCAAACTATATGGGAGTGGAAATAAACTG |
| | | ATAACAGTCGGGAGTTCCAAATATCATCAA |
| | | TCTTTTGTGCCGAGTCCAGGAACACGACCG |
| | | CAGATAAATGGCCAGTCCGGACGGATTGAT |
| | | TTTCATTGGTTGATCTTGGATCCCAATGAT |
| | | ACAGTTACTTTTAGTTTCAATGGGGCTTTC |
| | | ATAGCTCCAAATCGTGCCAGCTTCTTGAGG |
| | | GGAAAGTCCATGGGGATCCAGAGCGATGTG |
| | | CAGGTTGATGCCAATTGCGAAGGGGAATGC |
| | | TACCACAGTGGAGGGACTATAACAAGCAGA |
| | | TTGCCTTTTCAAAACATCAATAGCAGAGCA |
| | | GTTGGCAAATGCCCAAGATATGTAAAACAG |
| | | GAAAGTTTATTATTGGCAACTGGGATGAAG |
| | | AACGTTCCCGAACCTTCCAAAAAAAGGAAA |
| | | AAAAGAGGCCTGTTTGGCGCTATAGCAGGG |
| | | TTTATTGAAAATGGTTGGGAAGGTCTGGTC |
| | | GACGGGTGGTACGGTTTCAGGCATCAGAAT |
| | | GCACAAGGAGAAGGAACTGCAGCAGACTAC |
| | | AAAAGCACCCAATCGGCAATTGATCAGATA |
| | | ACCGGAAAGTTAAATAGACTCATTGAGAAA |
| | | ACCAACCAGCAATTTGAGCTAATAGATAAT |
| | | GAATTCACTGAGGTGGAAAAGCAGATTGGC |
| | | AATTTAATTAACTGGACCAAAGACTCCATC |
| | | ACAGAAGTATGGTCTTACAATGCTGAACTT |
| | | CTTGTGGCAATGGAAAACCAGCACACTATT |
| | | GATTTGGCTGATTCAGAGATGAACAAGCTG |
| | | TATGAGCGAGTGAGGAAACAATTAAGGGAA |
| | | AATGCTGAAGAGGATGGCACTGGTTGCTTT |
| | | GAAATTTTTCATAAATGTGACGATGATTGT |
| | | ATGGCTAGTATAAGGAACAATACTTATGAT |
| | | CACAGCAAATACAGAGAAGAAGCGATGCAA |
| | | AATAGAATACAAATTGACCCAGTCAAATTG |
| | | AGTAGTGGCTACAAAGATGTGATACTTTGG |
| | | TTTAGCTTCGGGGCATCATGCTTTTTGCTT |
| | | CTTGCCATTGCAATGGGCCTTGTTTTCATA |
| | | TGTGTGAAGAACGGAAACATGCGGTGCACT |
| | | ATTTGTATATAA |
| 41 | Envelope; RRV | AGTGTAACAGAGCACTTTAATGTGTATAAG |
| | | GCTACTAGACCATACCTAGCACATTGCGCC |
| | | GATTGCGGGGACGGGTACTTCTGCTATAGC |
| | | CCAGTTGCTATCGAGGAGATCCGAGATGAG |
| | | GCGTCTGATGGCATGCTTAAGATCCAAGTC |
| | | TCCGCCCAAATAGGTCTGGACAAGGCAGGC |
| | | ACCCACGCCCACACGAAGCTCCGATATATG |
| | | GCTGGTCATGATGTTCAGGAATCTAAGAGA |
| | | GATTCCTTGAGGGTGTACACGTCCGCAGCG |
| | | TGCTCCATACATGGGACGATGGGACACTTC |
| | | ATCGTCGCACACTGTCCACCAGGCGACTAC |
| | | CTCAAGGTTTCGTTCGAGGACGCAGATTCG |
| | | CACGTGAAGGCATGTAAGGTCCAATACAAG |
| | | CACAATCCATTGCCGGTGGGTAGAGAGAAG |
| | | TTCGTGGTTAGACCACACTTTGGCGTAGAG |
| | | CTGCCATGCACCTCATACCAGCTGACAACG |
| | | GCTCCCACCGACGAGGAGATTGACATGCAT |
| | | ACACCGCCAGATATACCGGATCGCACCCTG |
| | | CTATCACAGACGGCGGGCAACGTCAAAATA |
| | | ACAGCAGGCGGCAGGACTATCAGGTACAAC |
| | | TGTACCTGCGGCCGTGACAACGTAGGCACT |
| | | ACCAGTACTGACAAGACCATCAACACATGC |
| | | AAGATTGACCAATGCCATGCTGCCGTCACC |
| | | AGCCATGACAAATGGCAATTTACCTCTCCA |
| | | TTTGTTCCCAGGGCTGATCAGACAGCTAGG |
| | | AAAGGCAAGGTACACGTTCCGTTCCCTCTG |
| | | ACTAACGTCACCTGCCGAGTGCCGTTGGCT |
| | | CGAGCGCCGGATGCCACCTATGGTAAGAAG |
| | | GAGGTGACCCTGAGATTACACCCAGATCAT |
| | | CCGACGCTCTTCTCCTATAGGAGTTTAGGA |
| | | GCCGAACCGCACCCGTACGAGGAATGGGTT |
| | | GACAAGTTCTCTGAGCGCATCATCCCAGTG |
| | | ACGGAAGAAGGGATTGAGTACCAGTGGGGC |
| | | AACAACCCGCCGGTCTGCCTGTGGGCGCAA |
| | | CTGACGACCGAGGGCAAACCCCATGGCTGG |
| | | CCACATGAAATCATTCAGTACTATTATGGA |
| | | CTATACCCCGCCGCCACTATTGCCGCAGTA |
| | | TCCGGGGCGAGTCTGATGGCCCTCCTAACT |
| | | CTGGCGGCCACATGCTGCATGCTGGCCACC |
| | | GCGAGGAGAAAGTGCCTAACACCGTACGCC |
| | | CTGACGCCAGGAGCGGTGGTACCGTTGACA |
| | | CTGGGGCTGCTTTGCTGCGCACCGAGGGCG |
| | | AATGCA |
| 42 | Envelope; MLV 10A1 | ATGGAAGGTCCAGCGTTCTCAAAACCCCTT |
| | | AAAGATAAGATTAACCCGTGGAAGTCCTTA |
| | | ATGGTCATGGGGGTCTATTTAAGAGTAGGG |
| | | ATGGCAGAGAGCCCCCATCAGGTCTTTAAT |
| | | GTAACCTGGAGAGTCACCAACCTGATGACT |
| | | GGGCGTACCGCCAATGCCACCTCCCTTTTA |
| | | GGAACTGTACAAGATGCCTTCCCAAGATTA |
| | | TATTTTGATCTATGTGATCTGGTCGGAGAA |
| | | GAGTGGGACCCTTCAGACCAGGAACCATAT |
| | | GTCGGGTATGGCTGCAAATACCCCGGAGGG |
| | | AGAAAGCGGACCCGGACTTTTGACTTTTAC |
| | | GTGTGCCCTGGGCATACCGTAAAATCGGGG |
| | | TGTGGGGGGCCAAGAGAGGGCTACTGTGGT |
| | | GAATGGGGTTGTGAAACCACCGGACAGGCT |
| | | TACTGGAAGCCCACATCATCATGGGACCTA |
| | | ATCTCCCTTAAGCGCGGTAACACCCCCTGG |
| | | GACACGGGATGCTCCAAAATGGCTTGTGGC |
| | | CCCTGCTACGACCTCTCCAAAGTATCCAAT |
| | | TCCTTCCAAGGGGCTACTCGAGGGGGCAGA |
| | | TGCAACCCTCTAGTCCTAGAATTCACTGAT |
| | | GCAGGAAAAAAGGCTAATTGGGACGGGCCC |
| | | AAATCGTGGGGACTGAGACTGTACCGGACA |
| | | GGAACAGATCCTATTACCATGTTCTCCCTG |
| | | ACCCGCCAGGTCCTCAATATAGGGCCCCGC |
| | | ATCCCCATTGGGCCTAATCCCGTGATCACT |
| | | GGTCAACTACCCCCCTCCCGACCCGTGCAG |
| | | ATCAGGCTCCCCAGGCCTCCTCAGCCTCCT |
| | | CCTACAGGCGCAGCCTCTATAGTCCCTGAG |
| | | ACTGCCCCACCTTCTCAACAACCTGGGACG |
| | | GGAGACAGGCTGCTAAACCTGGTAGAAGGA |
| | | GCCTATCAGGCGCTTAACCTCACCAATCCC |
| | | GACAAGACCCAAGAATGTTGGCTGTGCTTA |
| | | GTGTCGGGACCTCCTTATTACGAAGGAGTA |
| | | GCGGTCGTGGGCACTTATACCAATCATTCT |
| | | ACCGCCCCGGCCAGCTGTACGGCCACTTCC |
| | | CAACATAAGCTTACCCTATCTGAAGTGACA |
| | | GGACAGGGCCTATGCATGGGAGCACTACCT |
| | | AAAACTCACCAGGCCTTATGTAACACCACC |
| | | CAAAGTGCCGGCTCAGGATCCTACTACCTT |
| | | GCAGCACCCGCTGGAACAATGTGGGCTTGT |
| | | AGCACTGGATTGACTCCCTGCTTGTCCACC |
| | | ACGATGCTCAATCTAACCACAGACTATTGT |
| | | GTATTAGTTGAGCTCTGGCCCAGAATAATT |
| | | TACCACTCCCCCGATTATATGTATGGTCAG |
| | | CTTGAACAGCGTACCAAATATAAGAGGGAG |
| | | CCAGTATCGTTGACCCTGGCCCTTCTGCTA |
| | | GGAGGATT |
| | | AACCATGGGAGGGATTGCAGCTGGAATAGG |
| | | GACGGGGACCACTGCCCTAATCAAAACCCA |
| | | GCAGTTTGAGCAGCTTCACGCCGCTATCCA |
| | | GACAGACCTCAACGAAGTCGAAAAATCAAT |
| | | TACCAACCTAGAAAAGTCACTGACCTCGTT |
| | | GTCTGAAGTAGTCCTACAGAACCGAAGAGG |
| | | CCTAGATTTGCTCTTCCTAAAAGAGGGAGG |
| | | TCTCTGCGCAGCCCTAAAAGAAGAATGTTG |
| | | TTTTTATGCAGACCACACGGGACTAGTGAG |
| | | AGACAGCATGGCCAAACTAAGGGAAAGGCT |
| | | TAATCAGAGACAAAAACTATTTGAGTCAGG |
| | | CCAAGGTTGGTTCGAAGGGCAGTTTAATAG |
| | | ATCCCCCTGGTTTACCACCTTAATCTCCAC |
| | | CATCATGGGACCTCTAATAGTACTCTTACT |
| | | GATCTTACTCTTTGGACCCTGCATTCTCAA |
| | | TCGATTGGTCCAATTTGTTAAAGACAGGAT |
| | | CTCAGTGGTCCAGGCTCTGGTTTTGACTCA |
| | | ACAATATCACCAGCTAAAACCTATAGAGTA |
| | | CGAGCCATGA |
| 43 | Envelope; Ebola | ATGGGTGTTACAGGAATATTGCAGTTACCT |
| | | CGTGATCGATTCAAGAGGACATCATTCTTT |
| | | CTTTGGGTAATTATCCTTTTCCAAAGAACA |
| | | TTTTCCATCCCACTTGGAGTCATCCACAAT |
| | | AGCACATTACAGGTTAGTGATGTCGACAAA |
| | | CTGGTTTGCCGTGACAAACTGTCATCCACA |
| | | AATCAATTGAGATCAGTTGGACTGAATCTC |
| | | GAAGGGAATGGAGTGGCAACTGACGTGCCA |
| | | TCTGCAACTAAAAGATGGGGCTTCAGGTCC |
| | | GGTGTCCCACCAAAGGTGGTCAATTATGAA |
| | | GCTGGTGAATGGGCTGAAAACTGCTACAAT |
| | | CTTGAAATCAAAAAACCTGACGGGAGTGAG |
| | | TGTCTACCAGCAGCGCCAGACGGGATTCGG |
| | | GGCTTCCCCCGGTGCCGGTATGTGCACAAA |
| | | GTATCAGGAACGGGACCGTGTGCCGGAGAC |
| | | TTTGCCTTCCACAAAGAGGGTGCTTTCTTC |
| | | CTGTATGACCGACTTGCTTCCACAGTTATC |
| | | TACCGAGGAACGACTTTCGCTGAAGGTGTC |
| | | GTTGCATTTCTGATACTGCCCCAAGCTAAG |
| | | AAGGACTTCTTCAGCTCACACCCCTTGAGA |
| | | GAGCCGGTCAATGCAACGGAGGACCCGTCT |
| | | AGTGGCTACTATTCTACCACAATTAGATAT |
| | | CAAGCTACCGGTTTTGGAACCAATGAGACA |
| | | GAGTATTTGTTCGAGGTTGACAATTTGACC |
| | | TACGTCCAACTTGAATCAAGATTCACACCA |
| | | CAGTTTCTGCTCCAGCTGAATGAGACAATA |
| | | TATACAAGTGGGAAAAGGAGCAATACCACG |
| | | GGAAAACTAATTTGGAAGGTCAACCCCGAA |
| | | ATTGATACAACAATCGGGGAGTGGGCCTTC |
| | | TGGGAAACTAAAAAAACCTCACTAGAAAAA |
| | | TTCGCAGTGAAGAGTTGTCTTTCACAGCTG |
| | | TATCAAACAGAGCCAAAAACATCAGTGGTC |
| | | AGAGTCCGGCGCGAACTTCTTCCGACCCAG |
| | | GGACCAACACAACAACTGAAGACCACAAAA |
| | | TCATGGCTTCAGAAAATTCCTCTGCAATGG |
| | | TTCAAGTGCACAGTCAAGGAAGGGAAGCTG |
| | | CAGTGTCGCATCTGACAACCCTTGCCACAA |
| | | TCTCCACGAGTCCTCAACCCCCCACAACCA |
| | | AACCAGGTCCGGACAACAGCACCCACAATA |
| | | CACCCGTGTATAAACTTGACATCTCTGAGG |
| | | CAACTCAAGTTGAACAACATCACCGCAGAA |
| | | CAGACAACGACAGCACAGCCTCCGACACTC |
| | | CCCCCGCCACGACCGCAGCCGGACCCCTAA |
| | | AAGCAGAGAACACCAACACGAGCAAGGGTA |
| | | CCGACCTCCTGGACCCCGCCACCACAACAA |
| | | GTCCCCAAAACCACAGCGAGACCGCTGGCA |
| | | ACAACAACACTCATCACCAAGATACCGGAG |
| | | AAGAGAGTGCCAGCAGCGGGAAGCTAGGCT |
| | | TAATTACCAATACTATTGCTGGAGTCGCAG |
| | | GACTGATCACAGGCGGGAGGAGAGCTCGAA |
| | | GAGAAGCAATTGTCAATGCTCAACCCAAAT |
| | | GCAACCCTAATTTACATTACTGGACTACTC |
| | | AGGATGAAGGTGCTGCAATCGGACTGGCCT |
| | | GGATACCATATTTCGGGCCAGCAGCCGAGG |
| | | GAATTTACATAGAGGGGCTGATGCACAATC |
| | | AAGATGGTTTAATCTGTGGGTTGAGACAGC |
| | | TGGCCAACGAGACGACTCAAGCTCTTCAAC |
| | | TGTTCCTGAGAGCCACAACCGAGCTACGCA |
| | | CCTTTTCAATCCTCAACCGTAAGGCAATTG |
| | | ATTTCTTGCTGCAGCGATGGGGCGGCACAT |
| | | GCCACATTTTGGGACCGGACTGCTGTATCG |
| | | AACCACATGATTGGACCAAGAACATAACAG |
| | | ACAAAATTGATCAGATTATTCATGATTTTG |
| | | TTGATAAAACCCTTCCGGACCAGGGGGACA |
| | | ATGACAATTGGTGGACAGGATGGAGACAAT |
| | | GGATACCGGCAGGTATTGGAGTTACAGGCG |
| | | TTATAATTGCAGTTATCGCTTTATTCTGTA |
| | | TATGCAAATTTGTCTTTTAG |
| 44 | Rous Sarcoma virus | GTAGTCTTATGCAATACTCTTGTAGTCTTG |
| | (RSV) promoter | CAACATGGTAACGATGAGTTAGCAACATGC |
| | | CTTACAAGGAGAGAAAAAGCACCGTGCATG |
| | | CCGATTGGTGGAAGTAAGGTGGTACGATCG |
| | | TGCCTTATTAGGAAGGCAACAGACGGGTCT |
| | | GACATGGATTGGACGAACCACTGAATTGCC |
| | | GCATTGCAGAGATATTGTATTTAAGTGCCT |
| | | AGCTCGATACAATAAACG |
| 45 | 5′ Long terminal repeat | GGTCTCTCTGGTTAGACCAGATCTGAGCCT |
| | (LTR) | GGGAGCTCTCTGGCTAACTAGGGAACCCAC |
| | | TGCTTAAGCCTCAATAAAGCTTGCCTTGAG |
| | | TGCTTCAAGTAGTGTGTGCCCGTCTGTTGT |
| | | GTGACTCTGGTAACTAGAGATCCCTCAGAC |
| | | CCTTTTAGTCAGTGTGGAAAATCTCTAGCA |
| 46 | Psi Packaging signal | TACGCCAAAAATTTTGACTAGCGGAGGCTA |
| | | GAAGGAGAGAG |
| 47 | Helper/Rev response | AGGAGCTTTGTTCCTTGGGTTCTTGGGAGC |
| | element (RRE) | AGCAGGAAGCACTATGGGCGCAGCCTCAAT |
| | | GACGCTGACGGTACAGGCCAGACAATTATT |
| | | GTCTGGTATAGTGCAGCAGCAGAACAATTT |
| | | GCTGAGGGCTATTGAGGCGCAACAGCATCT |
| | | GTTGCAACTCACAGTCTGGGGCATCAAGCA |
| | | GCTCCAGGCAAGAATCCTGGCTGTGGAAAG |
| | | ATACCTAAAGGATCAACAGCTCC |
| 48 | Central polypurine tract | TTTTAAAAGAAAAGGGGGGATTGGGGGGTA |
| | (cPPT) | CAGTGCAGGGGAAAGAATAGTAGACATAAT |
| | | AGCAACAGACATACAAACTAAAGAATTACA |
| | | AAAACAAATTACAAAATTCAAAATTTTA |
| 49 | Polymerase III shRNA | GAACGCTGACGTCATCAACCCGCTCCAAGG |
| | promoters; H1 promoter | AATCGCGGGCCCAGTGTCACTAGGCGGGAA |
| | | CACCCAGCGCGCGTGCGCCCTGGCAGGAAG |
| | | ATGGCTGTGAGGGACAGGGGAGTGGCGCCC |
| | | TGCAATATTTGCATGTCGCTATGTGTTCTG |
| | | GGAAATCACCATAAACGTGAAATGTCTTTG |
| | | GATTTGGGAATCTTATAAGTTCTGTATGAG |
| | | ACCACTT |
| 50 | FDPS shRNA sequence | GTCCTGGAGTACAATGCCATTCTCGAGAAT |
| | #1 | GGCATTGTACTCCAGGACTTTTT |
| 51 | FDPS shRNA sequence | GCAGGATTTCGTTCAGCACTTCTCGAGAAG |
| | #2 | TGCTGAACGAAATCCTGCTTTTT |
| 52 | FDPS shRNA sequence | GCCATGTACATGGCAGGAATTCTCGAGAAT |
| | #3 | TCCTGCCATGTACATGGCTTTTT |
| 53 | FDPS shRNA sequence | GCAGAAGGAGGCTGAGAAAGTCTCGAGACT |
| | #4 | TTCTCAGCCTCCTTCTGCTTTTT |
| 54 | Long WPRE sequence | AATCAACCTCTGATTACAAAATTTGTGAAA |
| | | GATTGACTGGTATTCTTAACTATGTTGCTC |
| | | CTTTTACGCTATGTGGATACGCTGCTTTAA |
| | | TGCCTTTGTATCATGCTATTGCTTCCCGTA |
| | | TGGCTTTCATTTTCTCCTCCTTGTATAAAT |
| | | CCTGGTTGCTGTCTCTTTATGAGGAGTTGT |
| | | GGCCCGTTGTCAGGCAACGTGGCGTGGTGT |
| | | GCACTGTGTTTGCTGACGCAACCCCCACTG |
| | | GTTGGGGCATTGCCACCACCTGTCAGCTCC |
| | | TTTCCGGGACTTTCGCTTTCCCCCTCCCTA |
| | | TTGCCACGGCGGAACTCATCGCCGCCTGCC |
| | | TTGCCCGCTGCTGGACAGGGGCTCGGCTGT |
| | | TGGGCACTGACAATTCCGTGGTGTTGTCGG |
| | | GGAAATCATCGTCCTTTCCTTGGCTGCTCG |
| | | CCTGTGTTGCCACCTGGATTCTGCGCGGGA |
| | | CGTCCTTCTGCTACGTCCCTTCGGCCCTCA |
| | | ATCCAGCGGACCTTCCTTCCCGCGGCCTGC |
| | | TGCCGGCTCTGCGGCCTCTTCCGCGTCTTC |
| | | GCCTTCGCCCTCAGACGAGTCGGATCTCCC |
| | | TTTGGGCCGCCTCCCCGCCT |
| 55 | 3′ delta LTR | TGGAAGGGCTAATTCACTCCCAACGAAGAT |
| | | AAGATCTGCTTTTTGCTTGTACTGGGTCTC |
| | | TCTGGTTAGACCAGATCTGAGCCTGGGAGC |
| | | TCTCTGGCTAACTAGGGAACCCACTGCTTA |
| | | AGCCTCAATAAAGCTTGCCTTGAGTGCTTC |
| | | AAGTAGTGTGTGCCCGTCTGTTGTGTGACT |
| | | CTGGTAACTAGAGATCCCTCAGACCCTTTT |
| | | AGTCAGTGTGGAAAATCTCTAGCAGTAGTA |
| | | GTTCATGTCA |
| 56 | Helper/Rev; Chicken beta | GCTATTACCATGGGTCGAGGTGAGCCCCAC |
| | actin (CAG) promoter; | GTTCTGCTTCACTCTCCCCATCTCCCCCCC |
| | Transcription | CTCCCCACCCCCAATTTTGTATTTATTTAT |
| | | TTTTTAATTATTTTGTGCAGCGATGGGGGC |
| | | GGGGGGGGGGGGGGCGCGCGCCAGGCGGGG |
| | | CGGGGCGGGGCGAGGGGCGGGGCGGGGCGA |
| | | GGCGGAGAGGTGCGGCGGCAGCCAATCAGA |
| | | GCGGCGCGCTCCGAAAGTTTCCTTTTATGG |
| | | CGAGGCGGCGGCGGCGGCGGCCCTATAAAA |
| | | AGCGAAGCGCGCGGCGGGCG |
| 57 | Helper/Rev; HIV Gag; | ATGGGTGCGAGAGCGTCAGTATTAAGCGGG |
| | Viral capsid | GGAGAATTAGATCGATGGGAAAAAATTCGG |
| | | TTAAGGCCAGGGGGAAAGAAAAAATATAAA |
| | | TTAAAACATATAGTATGGGCAAGCAGGGAG |
| | | CTAGAACGATTCGCAGTTAATCCTGGCCTG |
| | | TTAGAAACATCAGAAGGCTGTAGACAAATA |
| | | CTGGGACAGCTACAACCATCCCTTCAGACA |
| | | GGATCAGAAGAACTTAGATCATTATATAAT |
| | | ACAGTAGCAACCCTCTATTGTGTGCATCAA |
| | | AGGATAGAGATAAAAGACACCAAGGAAGCT |
| | | TTAGACAAGATAGAGGAAGAGCAAAACAAA |
| | | AGTAAGAAAAAAGCACAGCAAGCAGCAGCT |
| | | GACACAGGACACAGCAATCAGGTCAGCCAA |
| | | AATTACCCTATAGTGCAGAACATCCAGGGG |
| | | CAAATGGTACATCAGGCCATATCACCTAGA |
| | | ACTTTAAATGCATGGGTAAAAGTAGTAGAA |
| | | GAGAAGGCTTTCAGCCCAGAAGTGATACCC |
| | | ATGTTTTCAGCATTATCAGAAGGAGCCACC |
| | | CCACAAGATTTAAACACCATGCTAAACACA |
| | | GTGGGGGGACATCAAGCAGCCATGCAAATG |
| | | TTAAAAGAGACCATCAATGAGGAAGCTGCA |
| | | GAATGGGATAGAGTGCATCCAGTGCATGCA |
| | | GGGCCTATTGCACCAGGCCAGATGAGAGAA |
| | | CCAAGGGGAAGTGACATAGCAGGAACTACT |
| | | AGTACCCTTCAGGAACAAATAGGATGGATG |
| | | ACACATAATCCACCTATCCCAGTAGGAGAA |
| | | ATCTATAAAAGATGGATAATCCTGGGATTA |
| | | AATAAAATAGTAAGAATGTATAGCCCTACC |
| | | AGCATTCTGGACATAAGACAAGGACCAAAG |
| | | GAACCCTTTAGAGACTATGTAGACCGATTC |
| | | TATAAAACTCTAAGAGCCGAGCAAGCTTCA |
| | | CAAGAGGTAAAAAATTGGATGACAGAAACC |
| | | TTGTTGGTCCAAAATGCGAACCCAGATTGT |
| | | AAGACTATTTTAAAAGCATTGGGACCAGGA |
| | | GCGACACTAGAAGAAATGATGACAGCATGT |
| | | CAGGGAGTGGGGGGACCCGGCCATAAAGCA |
| | | AGAGTTTTGGCTGAAGCAATGAGCCAAGTA |
| | | ACAAATCCAGCTACCATAATGATACAGAAA |
| | | GGCAATTTTAGGAACCAAAGAAAGACTGTT |
| | | AAGTGTTTCAATTGTGGCAAAGAAGGGCAC |
| | | ATAGCCAAAAATTGCAGGGCCCCTAGGAAA |
| | | AAGGGCTGTTGGAAATGTGGAAAGGAAGGA |
| | | CACCAAATGAAAGATTGTACTGAGAGACAG |
| | | GCTAATTTTTTAGGGAAGATCTGGCCTTCC |
| | | CACAAGGGAAGGCCAGGGAATTTTCTTCAG |
| | | AGCAGACCAGAGCCAACAGCCCCACCAGAA |
| | | GAGAGCTTCAGGTTTGGGGAAGAGACAACA |
| | | ACTCCCTCTCAGAAGCAGGAGCCGATAGAC |
| | | AAGGAACTGTATCCTTTAGCTTCCCTCAGA |
| | | TCACTCTTTGGCAGCGACCCCTCGTCACAA |
| | | TAA |
| 58 | Helper/Rev; HIV Pol; | ATGAATTTGCCAGGAAGATGGAAACCAAAA |
| | Protease and reverse | ATGATAGGGGGAATTGGAGGTTTTATCAAA |
| | transcriptase | GTAGGACAGTATGATCAGATACTCATAGAA |
| | | ATCTGCGGACATAAAGCTATAGGTACAGTA |
| | | TTAGTAGGACCTACACCTGTCAACATAATT |
| | | GGAAGAAATCTGTTGACTCAGATTGGCTGC |
| | | ACTTTAAATTTTCCCATTAGTCCTATTGAG |
| | | ACTGTACCAGTAAAATTAAAGCCAGGAATG |
| | | GATGGCCCAAAAGTTAAACAATGGCCATTG |
| | | ACAGAAGAAAAAATAAAAGCATTAGTAGAA |
| | | ATTTGTACAGAAATGGAAAAGGAAGGAAAA |
| | | ATTTCAAAAATTGGGCCTGAAAATCCATAC |
| | | AATACTCCAGTATTTGCCATAAAGAAAAAA |
| | | GACAGTACTAAATGGAGAAAATTAGTAGAT |
| | | TTCAGAGAACTTAATAAGAGAACTCAAGAT |
| | | TTCTGGGAAGTTCAATTAGGAATACCACAT |
| | | CCTGCAGGGTTAAAACAGAAAAAATCAGTA |
| | | ACAGTACTGGATGTGGGCGATGCATATTTT |
| | | TCAGTTCCCTTAGATAAAGACTTCAGGAAG |
| | | TATACTGCATTTACCATACCTAGTATAAAC |
| | | AATGAGACACCAGGGATTAGATATCAGTAC |
| | | AATGTGCTTCCACAGGGATGGAAAGGATCA |
| | | CCAGCAATATTCCAGTGTAGCATGACAAAA |
| | | ATCTTAGAGCCTTTTAGAAAACAAAATCCA |
| | | GACATAGTCATCTATCAATACATGGATGAT |
| | | TTGTATGTAGGATCTGACTTAGAAATAGGG |
| | | CAGCATAGAACAAAAATAGAGGAACTGAGA |
| | | CAACATCTGTTGAGGTGGGGATTTACCACA |
| | | CCAGACAAAAAACATCAGAAAGAACCTCCA |
| | | TTCCTTTGGATGGGTTATGAACTCCATCCT |
| | | GATAAATGGACAGTACAGCCTATAGTGCTG |
| | | CCAGAAAAGGACAGCTGGACTGTCAATGAC |
| | | ATACAGAAATTAGTGGGAAAATTGAATTGG |
| | | GCAAGTCAGATTTATGCAGGGATTAAAGTA |
| | | AGGCAATTATGTAAACTTCTTAGGGGAACC |
| | | AAAGCACTAACAGAAGTAGTACCACTAACA |
| | | GAAGAAGCAGAGCTAGAACTGGCAGAAAAC |
| | | AGGGAGATTCTAAAAGAACCGGTACATGGA |
| | | GTGTATTATGACCCATCAAAAGACTTAATA |
| | | GCAGAAATACAGAAGCAGGGGCAAGGCCAA |
| | | TGGACATATCAAATTTATCAAGAGCCATTT |
| | | AAAAATCTGAAAACAGGAAAATATGCAAGA |
| | | ATGAAGGGTGCCCACACTAATGATGTGAAA |
| | | CAATTAACAGAGGCAGTACAAAAAATAGCC |
| | | ACAGAAAGCATAGTAATATGGGGAAAGACT |
| | | CCTAAATTTAAATTACCCATACAAAAGGAA |
| | | ACATGGGAAGCATGGTGGACAGAGTATTGG |
| | | CAAGCCACCTGGATTCCTGAGTGGGAGTTT |
| | | GTCAATACCCCTCCCTTAGTGAAGTTATGG |
| | | TACCAGTTAGAGAAAGAACCCATAATAGGA |
| | | GCAGAAACTTTCTATGTAGATGGGGCAGCC |
| | | AATAGGGAAACTAAATTAGGAAAAGCAGGA |
| | | TATGTAACTGACAGAGGAAGACAAAAAGTT |
| | | GTCCCCCTAACGGACACAACAAATCAGAAG |
| | | ACTGAGTTACAAGCAATTCATCTAGCTTTG |
| | | CAGGATTCGGGATTAGAAGTAAACATAGTG |
| | | ACAGACTCACAATATGCATTGGGAATCATT |
| | | CAAGCACAACCAGATAAGAGTGAATCAGAG |
| | | TTAGTCAGTCAAATAATAGAGCAGTTAATA |
| | | AAAAAGGAAAAAGTCTACCTGGCATGGGTA |
| | | CCAGCACACAAAGGAATTGGAGGAAATGAA |
| | | CAAGTAGATGGGTTGGTCAGTGCTGGAATC |
| | | AGGAAAGTACTA |
| 59 | Helper Rev; HIV | TTTTTAGATGGAATAGATAAGGCCCAAGAA |
| | Integrase; Integration of | GAACATGAGAAATATCACAGTAATTGGAGA |
| | viral RNA | GCAATGGCTAGTGATTTTAACCTACCACCT |
| | | GTAGTAGCAAAAGAAATAGTAGCCAGCTGT |
| | | GATAAATGTCAGCTAAAAGGGGAAGCCATG |
| | | CATGGACAAGTAGACTGTAGCCCAGGAATA |
| | | TGGCAGCTAGATTGTACACATTTAGAAGGA |
| | | AAAGTTATCTTGGTAGCAGTTCATGTAGCC |
| | | AGTGGATATATAGAAGCAGAAGTAATTCCA |
| | | GCAGAGACAGGGCAAGAAACAGCATACTTC |
| | | CTCTTAAAATTAGCAGGAAGATGGCCAGTA |
| | | AAAACAGTACATACAGACAATGGCAGCAAT |
| | | TTCACCAGTACTACAGTTAAGGCCGCCTGT |
| | | TGGTGGGCGGGGATCAAGCAGGAATTTGGC |
| | | ATTCCCTACAATCCCCAAAGTCAAGGAGTA |
| | | ATAGAATCTATGAATAAAGAATTAAAGAAA |
| | | ATTATAGGACAGGTAAGAGATCAGGCTGAA |
| | | CATCTTAAGACAGCAGTACAAATGGCAGTA |
| | | TTCATCCACAATTTTAAAAGAAAAGGGGGG |
| | | ATTGGGGGGTACAGTGCAGGGGAAAGAATA |
| | | GTAGACATAATAGCAACAGACATACAAACT |
| | | AAAGAATTACAAAAACAAATTACAAAAATT |
| | | CAAAATTTTCGGGTTTATTACAGGGACAGC |
| | | AGAGATCCAGTTTGGAAAGGACCAGCAAAG |
| | | CTCCTCTGGAAAGGTGAAGGGGCAGTAGTA |
| | | ATACAAGATAATAGTGACATAAAAGTAGTG |
| | | CCAAGAAGAAAAGCAAAGATCATCAGGGAT |
| | | TATGGAAAACAGATGGCAGGTGATGATTGT |
| | | GTGGCAAGTAGACAGGATGAGGATTAA |
| 60 | Helper/Rev; HIV RRE; | AGGAGCTTTGTTCCTTGGGTTCTTGGGAGC |
| | Binds Rev element | AGCAGGAAGCACTATGGGCGCAGCGTCAAT |
| | | GACGCTGACGGTACAGGCCAGACAATTATT |
| | | GTCTGGTATAGTGCAGCAGCAGAACAATTT |
| | | GCTGAGGGCTATTGAGGCGCAACAGCATCT |
| | | GTTGCAACTCACAGTCTGGGGCATCAAGCA |
| | | GCTCCAGGCAAGAATCCTGGCTGTGGAAAG |
| | | ATACCTAAAGGATCAACAGCTCCT |
| 61 | Helper/Rev; HIV Rev; | ATGGCAGGAAGAAGCGGAGACAGCGACGAA |
| | Nuclear export and | GAACTCCTCAAGGCAGTCAGACTCATCAAG |
| | stabilize viral mRNA | TTTCTCTATCAAAGCAACCCACCTCCCAAT |
| | | CCCGAGGGGACCCGACAGGCCCGAAGGAAT |
| | | AGAAGAAGAAGGTGGAGAGAGAGACAGAGA |
| | | CAGATCCATTCGATTAGTGAACGGATCCTT |
| | | AGCACTTATCTGGGACGATCTGCGGAGCCT |
| | | GTGCCTCTTCAGCTACCACCGCTTGAGAGA |
| | | CTTACTCTTGATTGTAACGAGGATTGTGGA |
| | | ACTTCTGGGACGCAGGGGGTGGGAAGCCCT |
| | | CAAATATTGGTGGAATCTCCTACAATATTG |
| | | GAGTCAGGAGCTAAAGAATAG |
| 62 | Envelope; CMV | ACATTGATTATTGACTAGTTATTAATAGTA |
| | promoter; Transcription | ATCAATTACGGGGTCATTAGTTCATAGCCC |
| | | ATATATGGAGTTCCGCGTTACATAACTTAC |
| | | GGTAAATGGCCCGCCTGGCTGACCGCCCAA |
| | | CGACCCCCGCCCATTGACGTCAATAATGAC |
| | | GTATGTTCCCATAGTAACGCCAATAGGGAC |
| | | TTTCCATTGACGTCAATGGGTGGAGTATTT |
| | | ACGGTAAACTGCCCACTTGGCAGTACATCA |
| | | AGTGTATCATATGCCAAGTACGCCCCCTAT |
| | | TGACGTCAATGACGGTAAATGGCCCGCCTG |
| | | GCATTATGCCCAGTACATGACCTTATGGGA |
| | | CTTTCCTACTTGGCAGTACATCTACGTATT |
| | | AGTCATCGCTATTACCATGGTGATGCGGTT |
| | | TTGGCAGTACATCAATGGGCGTGGATAGCG |
| | | GTTTGACTCACGGGGATTTCCAAGTCTCCA |
| | | CCCCATTGACGTCAATGGGAGTTTGTTTTG |
| | | GCACCAAAATCAACGGGACTTTCCAAAATG |
| | | TCGTAACAACTCCGCCCCATTGACGCAAAT |
| | | GGGCGGTAGGCGTGTACGGTGGGAGGTCTA |
| | | TATAAGC |
| 63 | Envelope; VSV-G; | ATGAAGTGCCTTTTGTACTTAGCCTTTTTA |
| | Glycoprotein envelope- | TTCATTGGGGTGAATTGCAAGTTCACCATA |
| | cell entry | GTTTTTCCACACAACCAAAAAGGAAACTGG |
| | | AAAAATGTTCCTTCTAATTACCATTATTGC |
| | | CCGTCAAGCTCAGATTTAAATTGGCATAAT |
| | | GACTTAATAGGCACAGCCTTACAAGTCAAA |
| | | ATGCCCAAGAGTCACAAGGCTATTCAAGCA |
| | | GACGGTTGGATGTGTCATGCTTCCAAATGG |
| | | GTCACTACTTGTGATTTCCGCTGGTATGGA |
| | | CCGAAGTATATAACACATTCCATCCGATCC |
| | | TTCACTCCATCTGTAGAACAATGCAAGGAA |
| | | AGCATTGAACAAACGAAACAAGGAACTTGG |
| | | CTGAATCCAGGCTTCCCTCCTCAAAGTTGT |
| | | GGATATGCAACTGTGACGGATGCCGAAGCA |
| | | GTGATTGTCCAGGTGACTCCTCACCATGTG |
| | | CTGGTTGATGAATACACAGGAGAATGGGTT |
| | | GATTCACAGTTCATCAACGGAAAATGCAGC |
| | | AATTACATATGCCCCACTGTCCATAACTCT |
| | | ACAACCTGGCATTCTGACTATAAGGTCAAA |
| | | GGGCTATGTGATTCTAACCTCATTTCCATG |
| | | GACATCACCTTCTTCTCAGAGGACGGAGAG |
| | | CTATCATCCCTGGGAAAGGAGGGCACAGGG |
| | | TTCAGAAGTAACTACTTTGCTTATGAAACT |
| | | GGAGGCAAGGCCTGCAAAATGCAATACTGC |
| | | AAGCATTGGGGAGTCAGACTCCCATCAGGT |
| | | GTCTGGTTCGAGATGGCTGATAAGGATCTC |
| | | TTTGCTGCAGCCAGATTCCCTGAATGCCCA |
| | | GAAGGGTCAAGTATCTCTGCTCCATCTCAG |
| | | ACCTCAGTGGATGTAAGTCTAATTCAGGAC |
| | | GTTGAGAGGATCTTGGATTATTCCCTCTGC |
| | | CAAGAAACCTGGAGCAAAATCAGAGCGGGT |
| | | CTTCCAATCTCTCCAGTGGATCTCAGCTAT |
| | | CTTGCTCCTAAAAACCCAGGAACCGGTCCT |
| | | GCTTTCACCATAATCAATGGTACCCTAAAA |
| | | TACTTTGAGACCAGATACATCAGAGTCGAT |
| | | ATTGCTGCTCCAATCCTCTCAAGAATGGTC |
| | | GGAATGATCAGTGGAACTACCACAGAAAGG |
| | | GAACTGTGGGATGACTGGGCACCATATGAA |
| | | GACGTGGAAATTGGACCCAATGGAGTTCTG |
| | | AGGACCAGTTCAGGATATAAGTTTCCTTTA |
| | | TACATGATTGGACATGGTATGTTGGACTCC |
| | | GATCTTCATCTTAGCTCAAAGGCTCAGGTG |
| | | TTCGAACATCCTCACATTCAAGACGCTGCT |
| | | TCGCAACTTCCTGATGATGAGAGTTTATTT |
| | | TTTGGTGATACTGGGCTATCCAAAAATCCA |
| | | ATCGAGCTTGTAGAAGGTTGGTTCAGTAGT |
| | | TGGAAAAGCTCTATTGCCTCTTTTTTCTTT |
| | | ATCATAGGGTTAATCATTGGACTATTCTTG |
| | | GTTCTCCGAGTTGGTATCCATCTTTGCATT |
| | | AAATTAAAGCACACCAAGAAAAGACAGATT |
| | | TATACAGACATAGAGATGA |
| 64 | miR30 FDPS sequence #1 | AAGGTATATTGCTGTTGACAGTGAGCGACA |
| | | CTTTCTCAGCCTCCTTCTGCGTGAAGCCAC |
| | | AGATGGCAGAAGGAGGCTGAGAAAGTGCTG |
| | | CCTACTGCCTCGGACTTCAAGGGGCT |
| 65 | miR30 FDPS sequence #2 | AAGGTATATTGCTGTTGACAGTGAGCGACA |
| | | CTTTCTCAGCCTCCTTCTGCGTGAAGCCAC |
| | | AGATGGCAGAAGGGCTGAGAAAGTGCTGCC |
| | | TACTGCCTCGGACTTCAAGGGGCT |
| 66 | miR30 FDPS sequence #3 | TGCTGTTGACAGTGAGCGACTTTCTCAGCC |
| | | TCCTTCTGCGTGAAGCCACAGATGGCAGAA |
| | | GGAGGCTGAGAAAGTTGCCTACTGCCTCGG |
| | | A |
| 67 | DNA Fragment | GCGGCCGCCACATGGAATTCTGCAACAACT |
| | containing RRE and | GCTGTTTATCCATTTCAGAATTGGAGGAGC |
| | rabbit beta | TTTGTTCCTTGGGTTCTTGGGAGCAGCAGG |
| | globin poly A | AAGCACTATGGGCGCAGCGTCAATGACGCT |
| | | GACGGTACAGGCCAGACAATTATTGTCTGG |
| | | TATAGTGCAGCAGCAGAACAATTTGCTGAG |
| | | GGCTATTGAGGCGCAACAGCATCTGTTGCA |
| | | ACTCACAGTCTGGGGCATCAAGCAGCTCCA |
| | | GGCAAGAATCCTGGCTGTGGAAAGATACCT |
| | | AAAGGATCAACAGCTCCTGGGGATTTGGGG |
| | | TTGCTCTGGAAAACTCATTTGCACCACTGC |
| | | TGTGCCTTGGAATGCTAGTTGGAGTAATAA |
| | | ATCTCTGGAACAGATTTGGAATCACACGAC |
| | | CTGGATGGAGTGGGACAGAGAAATTAACAA |
| | | TTACACAAGCTTGCTAGCAGATCTTTTTCC |
| | | CTCTGCCAAAAATTATGGGGACATCATGAA |
| | | GCCCCTTGAGCATCTGACTTCTGGCTAATA |
| | | AAGGAAATTTATTTTCATTGCAATAGTGTG |
| | | TTGGAATTTTTTGTGTCTCTCACTCGGAAG |
| | | GACATATGGGAGGGCAAATCATTTAAAACA |
| | | TCAGAATGAGTATTTGGTTTAGAGTTTGGC |
| | | AACATATGCCATATGCTGGCTGCCATGAAC |
| | | AAAGGTGGCTATAAAGAGGTCATCAGTATA |
| | | TGAAACAGCCCCCTGCTGTCCATTCCTTAT |
| | | TCCATAGAAAAGCCTTGACTTGAGGTTAGA |
| | | TTTTTTTTATATTTTGTTTTGTGTTATTTT |
| | | TTTCTTTAACATCCCTAAAATTTTCCTTAC |
| | | ATGTTTTACTAGCCAGATTTTTCCTCCTCT |
| | | CCTGACTACTCCCAGTCATAGCTGTCCCTC |
| | | TTCTCTTATGAAGATCCCTCGACCTGCAGC |
| | | CCAAGCTTGGCGTAATCATGGTCATAGCTG |
| | | TTTCCTGTGTGAAATTGTTATCCGCTCACA |
| | | ATTCCACACAACATACGAGCCGGAAGCATA |
| | | AAGTGTAAAGCCTGGGGTGCCTAATGAGTG |
| | | AGCTAACTCACATTAATTGCGTTGCGCTCA |
| | | CTGCCCGCTTTCCAGTCGGGAAACCTGTCG |
| | | TGCCAGCGGATCCGCATCTCAATTAGTCAG |
| | | CAACCATAGTCCCGCCCCTAACTCCGCCCA |
| | | TCCCGCCCCTAACTCCGCCCAGTTCCGCCC |
| | | ATTCTCCGCCCCATGGCTGACTAATTTTTT |
| | | TTATTTATGCAGAGGCCGAGGCCGCCTCGG |
| | | CCTCTGAGCTATTCCAGAAGTAGTGAGGAG |
| | | GCTTTTTTGGAGGCCTACGAATGA |

While certain of the preferred embodiments have been described and specifically exemplified above, it is not intended that the disclosure be limited to such preferred embodiments. Various modifications may be made thereto without departing from the scope and spirit of the present embodiments described herein.

What is claimed is:

1. A modified NK cell, comprising an NK cell that expresses at least one T cell receptor, wherein the at least one T cell receptor comprises at least one γ chain and at least one δ chain.

2. The modified NK cell of claim 1, wherein the NK cell further comprises at least one NK cell receptor.

3. The modified NK cell of claim 1, wherein the NK cell further expresses a CD3 protein.

4. The modified NK cell of claim 1, wherein the at least one δ chain comprises any one or more of a δ1 chain, a δ2 chain, a δ3 chain, and a δ5 chain.

5. The modified NK cell of claim 4, wherein the at least one δ chain comprises a δ2 chain.

6. The modified NK cell of claim 1, wherein the at least one γ chain comprises a γ9 chain.

7. The modified NK cell of claim 1, wherein the at least one T cell receptor comprises at least one Vγ9Vδ2 T cell receptor.

8. The modified NK cell of claim 1, wherein the at least one T cell receptor does not express CD16.

9. The modified NK cell of claim 1, wherein the NK cell comprises an NK92 cell, a KHYG-1 cell, an NKL cell, an NKG cell, an NK-YS cell, a HANK-1 cell, or a YT cell.

10. The modified NK cell of claim 9, wherein the NK cell comprises an NK92 cell.

11. The modified NK cell of claim 1, wherein the NK cell comprises a primary NK cell.

12. A pharmaceutical composition for treating cancer, comprising a combination of:
an aminobisphosphonate drug; and
the modified NK cell of claim 1.

13. The pharmaceutical composition of claim 12, wherein the aminobisphosphonate drug comprises zoledronic acid.

14. The pharmaceutical composition of claim 12, wherein the combination comprises a fixed combination.

15. The pharmaceutical composition of claim 12, wherein the combination comprises a non-fixed combination.

16. The pharmaceutical composition of claim 12, further comprising an inhibitor of FDPS.

17. The pharmaceutical composition of claim 16, wherein the inhibitor of FDPS comprises a shRNA or a microRNA.

18. The pharmaceutical composition of claim 12, further comprising an antagonist of CD16.

19. The pharmaceutical composition of claim 18, wherein the antagonist of CD16 comprises an siRNA, shRNA or miRNA.

20. A method of treating cancer in a subject, comprising administering to the subject an effective amount of the modified NK cell of claim 1.

21. The method of claim 20, wherein the subject has at least one cancer selected from one or more of a carcinoma, a leukemia, a lymphoma, a sarcoma, a myeloma, a mesothelioma, a mixed type, a glioma, a neuroblastic tumor, or mixtures thereof.

22. The method of claim 20, further comprising administering an effective amount of an aminobisphosphonate drug to the subject.

23. The method of claim 22, wherein the aminobisphosphonate drug comprises zoledronic acid.

24. The method of claim 22, wherein the aminobisphosphonate drug is administered separately from the modified NK cell.

25. The method of claim 22, wherein the aminobisphosphonate drug is administered together with the modified NK cell.

26. The method of claim 20, further comprising administering an effective amount of an inhibitor of FDPS.

27. The method of claim 26, wherein the inhibitor of FDPS is administered separately with the modified NK cell.

28. The method of claim 26, wherein the inhibitor of FDPS is administered together with the modified NK cell.

29. The method of claim 26, wherein the inhibitor of FDPS comprises a shRNA or a microRNA.

30. The method of claim 20, further comprising administering an effective amount of an antagonist of CD16.

31. The method of claim 30, wherein the antagonist of CD16 is administered separately with the modified NK cell.

32. The method of claim 30, wherein the antagonist of CD16 is administered together with the modified NK cell.

33. The method of claim 30, wherein the antagonist of CD16 comprises an siRNA, shRNA or miRNA.

34. A method of treating cancer, comprising administering to the subject an effective amount of the pharmaceutical composition of claim 12.

35. The method of claim 34, wherein the subject has at least one cancer selected from one or more of a carcinoma, a leukemia, a lymphoma, a sarcoma, a myeloma, a mesothelioma, a mixed type, a glioma, a neuroblastic tumor, or mixtures thereof.

36. The method of claim 34, wherein the aminobisphosphonate drug comprises zoledronic acid.

37. The method of claim 34, further comprising administering an effective amount of an inhibitor of FDPS.

38. The method of claim 37, wherein the inhibitor of FDPS is administered separately with the pharmaceutical composition.

39. The method of claim 37, wherein the inhibitor of FDPS is administer together with the pharmaceutical composition.

40. The method of claim 37, wherein the inhibitor of FDPS comprises a shRNA or a microRNA.

41. The method of claim 34, further comprising administering an effective amount of an inhibitor of CD16.

42. The method of claim 41, wherein the antagonist of CD16 is administered separately with the pharmaceutical composition.

43. The method of claim 41, wherein the antagonist of CD16 is administer together with the pharmaceutical composition.

44. The method of claim 41, wherein the antagonist of CD16 comprises a shRNA or a microRNA.

45. The modified NK cell of claim 1, wherein the NK cells has been selected for a lack of substantial CD16 expression.

46. The modified NK cell of claim 46, wherein the NK cells are selected by limited dilution and expansion of a monoclonal cell line.

47. The modified NK cell of claim 46, wherein CD16 expression is antagonized.

48. The modified NK cell of claim 46, wherein CD16 expression is antagonized with a small RNA that targets the CD16 mRNA and interferes with its expression.

49. The modified NK cell of claim 48, wherein the small RNA is an siRNA, miRNA or shRNA.

50. The modified NK cell of claim 47, wherein CD16 expression is antagonized by inactivating one or both copies of the CD16 gene with a gene editing system.

51. The modified NK cell of claim 50, wherein the gene editing system comprises a CRISPR/Cas system, zinc-finger nuclease system or Transcription Activator-Like Effector Nucleases (“TALENs”) system.

52. The modified NK cell of claim 47, wherein CD16 expression is antagonized by a targeted protein degrader.

53. The modified NK cell of claim 52, the targeted protein degrader includes a proteolysis targeting chimera molecules (“PROTAC”) degrader, molecular glue degrader or ubiquitin ligase modifier.

54. The modified NK cell of claim 1, wherein the NK cells are primary NK cells purified from the blood of a patient.

55. The modified NK cell of claim 54, wherein the NK cells are purified leukapheresis and/or density gradient centrifugation.

56. The modified NK cell of claim 55, wherein the purified NK cells are expanded ex vivo

57. The modified NK cell of claim 47, wherein the modified NK is further transduced with one or more viral integrating or non-integrating vectors encoding at least one T cell receptor, wherein the at least one T cell receptor comprises at least one γ chain and at least one δ chain.

58. The modified NK cell of claim 57, wherein the modified NK cells further comprises at least one NK cell receptor.

59. The modified NK cell of claim 58, wherein the modified NK cells further comprises at least at least one CD3 protein.

60. A pharmaceutical composition comprising a modified NK cell of any of claims 45-59.
